(19) United States
(12) Patent Application Publication
(10) Pub. No.: US 2026/0002185 A1
(43) Pub. Date: Jan. 1, 2026

(54) COMPOSITIONS AND METHODS FOR HETEROLOGOUS PRODUCTION OF INDIGOIDINE

(71) Applicant: SHREENIKA PIONEERING INC., San Diego, CA (US)

(72) Inventors: Jingyi LI, San Diego, CA (US); Jeffrey EHRHARDT, San Diego, CA (US); Jose A. AMAYA, San Diego, CA (US); Shawn Robert KULAKOWSKI, San Diego, CA (US); Cleo Lewis SCHOEPLEIN, San Diego, CA (US); Drew Taylor WAGNER, San Diego, CA (US); Ben MAI, San Diego, CA (US)

(21) Appl. No.: 19/252,828

(22) Filed: Jun. 27, 2025

Related U.S. Application Data
(60) Provisional application No. 63/665,631, filed on Jun. 28, 2024.

(51) Int. Cl. C12P 17/16; C12N 1/14; C12N 1/20; C12N 9/00; C12N 9/12; C12N 9/88; C12P 13/14; C12R 1/19; C12R 1/66; C12R 1/72; C12R 1/84; C12R 1/85

(52) U.S. Cl. CPC C12P 17/165; C12N 1/14; C12N 1/20; C12N 9/1288; C12N 9/88; C12N 9/93; C12P 13/14; C12Y 207/08007; C12Y 403/03; C12Y 603/02; C12R 2001/19; C12R 2001/66; C12R 2001/72; C12R 2001/84; C12R 2001/85

(57) ABSTRACT

The present disclosure relates to heterologous production of indigoidine. Provided herein are a heterologous host cell capable of expressing a polypeptide comprising at least about 70% sequence identity to any one of SEQ ID NOs: 1-5, wherein the heterologous host cell is capable of producing indigoidine; heterologous expression systems comprising the heterologous host cell; and nucleic acids encoding the polypeptide. Also provided are methods of making indigoidine, including cell-free methods, and compositions comprising indigoidine.

### SEQUENCE LISTING

The instant application contains a Sequence Listing which has been submitted electronically in XML format and is hereby incorporated by reference in its entirety. Said Sequence Listing XML, created on Jun. 26, 2025, is named “0300-0002WO1_SL.xml” and is 44,268 bytes in size. The sequence listing contained in this .XML file is part of the specification and is hereby incorporated by reference herein in its entirety.

### FIELD OF THE INVENTION

The present disclosure relates to heterologous production of indigoidine. Provided herein are a heterologous host cell capable of expressing a polypeptide comprising at least about 70% sequence identity to any one of SEQ ID NOs: 1-5, wherein the heterologous host cell is capable of producing indigoidine; heterologous expression systems comprising the heterologous host cell; and nucleic acids encoding the polypeptide. Also provided are methods of making indigoidine, including cell-free methods, and compositions comprising indigoidine.

### BACKGROUND

Indigoidine [5,5′-diamino-4,4′-dihydroxy-3,3′-diazadiphenoguinone-(2,2′)] is a natural blue pigment suitable for use as a sustainable dye and that possesses antioxidant and antimicrobial activity. In nature, indigoidine is produced by indigoidine synthetase (INDS) in several bacteria including Streptomyces lavendulae, Streptomyces chromofuscus, and Erwinia chrysanthemi (Takahashi et al., J. Biol. Chem. (2007) 282:9073-9081, 2007; Yu et al., J. Indus. Microbiol. Biotechnol. (2013) 40:159-168; Reverchon et al., J. Bacteriol. (2002) 184:654-665).

### SUMMARY OF THE INVENTION

In some embodiments, the present disclosure provides a heterologous host cell capable of expressing an indigoidine synthetase (INDS) including at least 70% sequence identity to any one of SEQ ID NOs: 1-5, wherein the heterologous host cell is capable of producing indigoidine.

In some embodiments, the present disclosure provides a heterologous expression system for the production of indigoidine, comprising a heterologous host cell comprising a heterologous nucleic acid, wherein the heterologous nucleic acid is capable of expressing an INDS comprising at least 70% sequence identity to any one of SEQ ID NOs: 1-5.

In some embodiments, the present disclosure provides a method of making indigoidine, comprising expressing an INDS comprising at least 70% sequence identity to any one of SEQ ID NOs: 1-5 in a heterologous host cell. In some embodiments, the heterologous host cell is in a culture medium, and wherein the method further includes isolating the indigoidine following the expressing. In some embodiments, the culture medium includes a carbon source, a supplement, or a combination thereof. In some embodiments, the carbon source includes glucose, glycerol, sucrose, galactose, acetate, succinate, lignocellulose, molasses, or combinations thereof. In some embodiments, the supplement includes glutamate, glutamine, pantothenate, casamino acid, thiamine, biotin, riboflavin, trace metals, vitamins, or combinations thereof.

In some embodiments, the present disclosure provides a nucleic acid encoding an INDS comprising at least 70% sequence identity to any one of SEQ ID NOs: 1-5, wherein the nucleic acid is operably linked to a heterologous regulatory element. In some embodiments, the heterologous regulatory element includes a promoter.

In some embodiments, the present disclosure provides a vector comprising the nucleic acid described herein. In some embodiments, the vector further includes one or both of: a nucleic acid encoding a PPTase; and a nucleic acid encoding an NRPS accessory protein. In some embodiments, the vector is a bacterial expression vector or a fungal expression vector.

In some embodiments, the present disclosure provides a heterologous host cell comprising the nucleic acid and/or the vector described herein.

In some embodiments, the INDS includes at least 80% sequence identity to any one of SEQ ID NOs: 1-5. In some embodiments, the INDS includes at least 90% sequence identity to any one of SEQ ID NOs: 1-5. In some embodiments, the INDS includes any one of SEQ ID NOs: 1-5.

In some embodiments, the heterologous host cell is further capable of expressing a 4′-phosphopantetheinyl transferase (PPTase), wherein the PPTase is capable of activating the INDS. In some embodiments, the PPTase is an endogenous PPTase. In some embodiments, the PPTase is an exogenous PPTase. In some embodiments, the exogenous PPTase includes at least 70% identity to any one of SEQ ID NOs: 9-21. In some embodiments, the exogenous PPTase includes at least 80% identity to any one of SEQ ID NOs: 9-21. In some embodiments, the exogenous PPTase includes at least 90% identity to any one of SEQ ID NOs: 9-21. In some embodiments, the exogenous PPTase includes any one of SEQ ID NOs: 9-21. In some embodiments, the exogenous PPTase includes SEQ ID NO: 9.

In some embodiments, the heterologous host cell is further capable of expressing a nonribosomal peptide synthetase (NRPS) accessory protein. In some embodiments, the NRPS accessory protein is an MbtH-like protein (MLP). In some embodiments, the MLP includes at least 70%, at least 80%, at least 90%, or 100% identity to any one of SEQ ID NOs: 22-24.

In some embodiments, the heterologous host cell is further capable of expressing an indigoidine transporter protein. In some embodiments, the indigoidine transporter protein comprises at least 70%, at least 80%, at least 90%, or 100% identity to any one of SEQ ID NOs: 25-32.

In some embodiments, the heterologous host cell includes one or more modifications to increase glutamate and/or glutamine production. In some embodiments, the heterologous host cell includes one or more modifications selected from: (a) increased levels of one or more of acetyl-coenzyme A synthetase, NADP-specific glutamate dehydrogenase, glutamine synthetase, isocitrate dehydrogenase, dihydrolipoyl dehydrogenase, phosphoenolpyruvate carboxylase, transcriptional repressor IclR, NAD(P) transhydrogenase subunit alpha, NAD(P) transhydrogenase subunit beta, Type III pantothenate kinase, glucose facilitated diffusion protein, glucokinase, or S-formylglutathione hydrolase; (b) decreased levels of one or more of D-lactate dehydrogenase, ubiquinone-dependent pyruvate dehydrogenase, aldehyde-alcohol dehydrogenase, phosphate acetyltransferase, acetate kinase, bifunctional glutamine synthetase adenylyltransferase, glutaminase 1, glutaminase 2, glutamate synthase [NADPH] large chain, glutamate synthase [NADPH] small chain, aerobic respiration control protein, 2-oxoglutarate dehydrogenase E1 component, dihydrolipoyllysine-residue succinyltransferase component of 2-oxoglutarate dehydrogenase complex, malate dehydrogenase, pyruvate kinase I, adenine deaminase, cytidine deaminase, guanine deaminase, outer membrane porin C, respiratory nitrate reductase 1 alpha chain, L-asparaginase 2, 2-iminobutanoate/2-iminopropanoate deaminase, adenosine deaminase, cytosine deaminase, dipeptide-binding protein, periplasmic oligopeptide-binding protein, fused glutamine synthetase deadenylase/glutamine synthetase adenylyltransferase, L-glutamine ABC transporter periplasmic binding protein, L-glutamine ABC transporter membrane subunit, L-glutamine ABC transporter ATP binding subunit, pseudouridine-5′-phosphate glycosidase, pseudouridine kinase, pseudouridine transporter, or tautomerase; and (c) altered expression of one or both of DNA gyrase subunit A and cytochrome d oxidase cydAB.

In some embodiments, the heterologous host cell is bacteria or fungi. In some embodiments, the heterologous host cell is bacteria, wherein the bacteria is Escherichia, Corynebacterium, Bacillus, Ralstonia, Zymomonas, or Staphylococcus. In some embodiments, the bacteria is Escherichia coli. In some embodiments, the heterologous host cell is fungi, wherein the fungi is Pichia, Saccharomyces, Candida, Yarrowia, or Aspergillus.

In some embodiments, the present disclosure provides a cell-free method of making indigoidine, comprising contacting an INDS comprising at least 70% sequence identity to any one of SEQ ID NOs: 1-5 with glutamine under conditions sufficient to produce indigoidine.

In some embodiments, the INDS is an activated INDS including a 4′-phosphopantetheine (PPT) group. In some embodiments, the method further includes, prior to the contacting, incubating the INDS with a PPTase and coenzyme A to form an activated INDS including a PPT group.

In some embodiments, the contacting is in the presence of ATP, MgCl₂, FMN, or combination thereof. In some embodiments, the contacting is further in the presence of an MLP, a polyphosphate kinase (PPK), ADP, inorganic polyphosphate, or combination thereof. In some embodiments, the INDS includes at least 80%, at least 90%, or 100% to any one of SEQ ID NOs: 1-5. In some embodiments, the method further includes isolating the indigoidine.

In some embodiments, the present disclosure provides composition comprising indigoidine made by the methods described herein. In some embodiments, the present disclosure provides a composition comprising indigoidine made by the cell-free method.

### BRIEF DESCRIPTION OF THE DRAWINGS

The following drawings form part of the present specification and are included to further demonstrate exemplary embodiments of certain aspects of the present disclosure.

FIG. 1 illustrates the indigoidine biosynthesis pathway, in which two molecules of L-glutamine are converted to indigoidine as described in embodiments herein.

FIG. 2 illustrates an exemplary indigoidine production pathway in E. coli from glucose or glycerol as the carbon source, as described in embodiments herein.

FIG. 3 illustrates a cell-free method for indigoidine production as described in embodiments herein.

### DETAILED DESCRIPTION OF THE INVENTION

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” means that the specific terms recited are representative examples and embodiments of the disclosure that are not intended to be limited to the specific examples referenced or cited unless explicitly stated otherwise.

As used herein, “about” can mean plus or minus 10% of the provided value. Where ranges are provided, they are inclusive of the boundary values. “About” can additionally or alternately mean either within 10% of the stated value, or within 5% of the stated value, or in some cases within 2.5% of the stated value; or, “about” can mean rounded to the nearest significant digit.

As used herein, “between” is a range inclusive of the ends of the range. For example, a number between x and y explicitly includes the numbers x and y, and any numbers that fall within x and y.

A “nucleic acid,” “nucleic acid molecule,” “nucleic acid sequence,” “nucleotide sequence,” “oligonucleotide,” or “polynucleotide” means a polymeric compound including covalently linked nucleotides. The term “nucleic acid” includes ribonucleic acid (RNA) and deoxyribonucleic acid (DNA), both of which may be single- or double-stranded. DNA includes, but is not limited to, complementary DNA (cDNA), genomic DNA, plasmid or vector DNA, and synthetic DNA. In some embodiments, the disclosure provides a nucleic acid encoding any one of the polypeptides described herein, e.g., an indigoidine synthetase.

A “gene” refers to an assembly of nucleotides that encode a polypeptide and includes cDNA and genomic DNA nucleic acid molecules. In some embodiments, “gene” also refers to a non-coding nucleic acid fragment that can act as a regulatory sequence preceding (i.e., 5′) and following (i.e., 3′) the coding sequence.

As used herein, the term “operably linked” means that a polynucleotide of interest, e.g., the polynucleotide encoding an indigoidine synthetase, is linked to the regulatory element in a manner that allows for expression of the polynucleotide. In some embodiments, the regulatory element is a promoter. In some embodiments, a nucleic acid expressing the polypeptide of interest is operably linked to a promoter on an expression vector.

As used herein, “promoter,” “promoter sequence,” or “promoter region” refers to a DNA regulatory region or polynucleotide capable of binding RNA polymerase and involved in initiating transcription of a downstream coding or non-coding sequence. In some embodiments, the promoter sequence includes the transcription initiation site and extends upstream to include the minimum number of bases or elements used to initiate transcription at levels detectable above background. In some embodiments, the promoter sequence includes a transcription initiation site, as well as protein binding domains responsible for the binding of RNA polymerase. Eukaryotic promoters typically contain “TATA” boxes and “CAT” boxes. Various promoters, including inducible promoters, may be used to drive expression of the polynucleotides described herein.

An “expression vector or vectors” (also referred to as “expression construct”) can be constructed to include one or more protein of interest-encoding nucleic acids (e.g., nucleic acid encoding an indigoidine synthetase described herein) operably linked to expression control sequences functional in a host organism. Expression vectors applicable for use in host organisms include, for example, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, yeast plasmids, yeast artificial chromosomes, and any other vectors specific for specific hosts of interest (such as E. coli and yeast). In some embodiments, the expression vector comprises a nucleic acid encoding a protein described herein, e.g., an indigoidine synthetase.

Additionally, the expression vectors can include one or more selectable marker genes and appropriate expression control sequences. Selectable marker genes also can be included that, for example, provide resistance to antibiotics or toxins, complement auxotrophic deficiencies, or supply critical nutrients not in the culture media. Expression control sequences can include constitutive and inducible promoters, transcription enhancers, transcription terminators, and the like. When two or more exogenous encoding nucleic acids (e.g., a gene encoding indigoidine synthetase and an additional gene encoding another enzyme that complements indigoidine biosynthesis pathway as described herein) are to be co-expressed, both nucleic acids can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The transformation of exogenous nucleic acid sequences involved in a metabolic or synthetic pathway can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the exogenous nucleic acid is expressed in a sufficient amount to produce the desired product, and it is further understood that expression levels can be optimized to obtain sufficient expression using methods well known in the art and as disclosed herein. The following vectors are provided by way of example; for bacterial host cells: pQE vectors (Qiagen), pBluescript plasmids, pNH vectors, pTWIST vectors (TWIST Bioscience), lambda-ZAP vectors (Stratagene); pTrc99a, pKK223-3, pDR540, and pRIT2T (Pharmacia); for eukaryotic host cells: pXT1, pSG5 (Stratagene), pSVK3, pBPV, pMSG, and pSVLSV40 (Pharmacia). However, any other plasmid or other vector may be used so long as it is compatible with the host cell.

The term “host cell” refers to a cell into which a recombinant expression vector has been introduced, or “host cell” may also refer to the progeny of such a cell. Because modifications may occur in succeeding generations, for example, due to mutation or environmental influences, the progeny may not be identical to the parent cell, but are still included within the scope of the term “host cell.” In some embodiments, the present disclosure provides a host cell comprising an expression vector that comprises a nucleic acid encoding an indigoidine synthetase described herein. In some embodiments, the host cell is a bacterial cell, a fungal cell, an algal cell, a cyanobacterial cell, or a plant cell.

A genetic alteration that makes an organism or cell “non-natural” can include, for example, modifications introducing expressible nucleic acids encoding metabolic polypeptides, other nucleic acid additions, nucleic acid deletions and/or other functional disruption of the organism's genetic material. Such modifications include, for example, coding regions and functional fragments thereof, for heterologous, homologous, or both heterologous and homologous polypeptides for the referenced species. Additional modifications include, for example, non-coding regulatory regions in which the modifications alter expression of a gene or operon.

A host cell or host organism capable of expressing or overexpressing a nucleic acid (e.g., a gene) or polypeptide (e.g., an enzyme), or engineered to express or overexpress the nucleic acid or to overexpress the polypeptide, has been genetically engineered (e.g., through recombinant DNA technology) to include a gene or nucleic acid sequence (which may encode the polypeptide) that it does not naturally include, or to express an endogenous gene at a level that exceeds its level of expression in a non-engineered host cell. As non-limiting examples, a host cell or host organism engineered to express or overexpress a nucleic acid or polypeptide can have any modifications that affect a coding sequence of a gene, the position of a gene on a chromosome or episome, or regulatory elements associated with a gene. A gene can also be overexpressed by increasing the copy number of a gene in the host cell or host organism. In some embodiments, overexpression of an endogenous gene comprises replacing the native promoter of the gene with a constitutive promoter that increases expression of the gene relative to expression in a control cell with the native promoter. In some embodiments, the constitutive promoter is heterologous.

Similarly, a host cell or host organism engineered to under-express (or to have reduced expression of) a nucleic acid (e.g., a gene) or to under-express a polypeptide (e.g., an enzyme) can have any modifications that affect a coding sequence of a gene, the position of a gene on a chromosome or episome, or regulatory elements associated with a gene. Specifically included are gene disruptions, which include any insertions, deletions, or sequence mutations into or of the gene or a portion of the gene that affect its expression or the activity of the encoded polypeptide. Gene disruptions include “knockout” mutations that eliminate expression of the gene. Modifications to under-express or downregulate a gene also include modifications to regulatory regions of the gene that can reduce its expression.

The term “exogenous” is intended to mean that the referenced molecule or the referenced activity is introduced into the host cell or host organism. The molecule can be introduced, for example, by introduction of an encoding nucleic acid into the host genetic material such as integration into a host chromosome or as non-chromosomal genetic material that may be introduced on a vehicle such as a plasmid. The term “exogenous nucleic acid” means a nucleic acid that is not naturally-occurring within the host cell or host organism. Exogenous nucleic acids may be derived from or identical to a naturally-occurring nucleic acid or it may be a non-naturally occurring nucleic acid. For example, a non-natural duplication of a naturally-occurring gene is considered to be an exogenous nucleic acid sequence. An exogenous nucleic acid can be introduced in an expressible form into the host cell or host organism. The term “exogenous activity” refers to an activity that is introduced into the host cell or host organism. The source can be, for example, a homologous or heterologous encoding nucleic acid that expresses the referenced activity following introduction into the host cell or host organism.

The term “endogenous” refers to a referenced molecule or activity that is naturally present in the host cell or host organism. Similarly, the term when used in reference to expression of an encoding nucleic acid refers to expression of an encoding nucleic acid contained within the host cell or host organism.

The term “heterologous” refers to a molecule or activity derived from a source other than the referenced species, whereas “homologous” refers to a molecule or activity derived from the host organism. Accordingly, exogenous expression of an encoding nucleic acid can utilize either or both of a heterologous or homologous encoding nucleic acid.

When used to refer to a genetic regulatory element, such as a promoter, operably linked to a gene, the term “homologous” refers to a regulatory element that is naturally operably linked to the referenced gene. In contrast, a “heterologous” regulatory element is not naturally found operably linked to the referenced gene, regardless of whether the regulatory element is naturally found in the host cell or host organism.

It is understood that more than one exogenous nucleic acid(s) can be introduced into the host cell or host organism on separate nucleic acid molecules, on polycistronic nucleic acid molecules, or combinations thereof, and still be considered as more than one exogenous nucleic acid. For example, as described herein, a host cell or host organism can be engineered to express at least two, three, four, five, six, seven, eight, nine, ten or more exogenous nucleic acids encoding desired proteins or enzymes of a particular biosynthesis pathway. In the case where two or more exogenous nucleic acids encoding a desired activity are introduced into a host cell or host organism, it is understood that the two or more exogenous nucleic acids can be introduced as a single nucleic acid, for example, on a single plasmid, on separate plasmids, or integrated into the host chromosome at a single site or multiple sites, and still be considered as two or more exogenous nucleic acids. Similarly, it is understood that more than two exogenous nucleic acids can be introduced into a host cell or host organism in any desired combination, for example, on a single plasmid, on separate plasmids, can be integrated into the host chromosome at a single site or multiple sites, and still be considered as two or more exogenous nucleic acids Thus, the number of referenced exogenous nucleic acids or biosynthetic activities refers to the number of encoding nucleic acids or the number of biosynthetic activities, not the number of separate nucleic acids introduced into the host cell or host organism.

Genes or nucleic acid sequences can be introduced stably or transiently into a host cell or host organism using techniques known in the art including, but not limited to, conjugation, electroporation, chemical transformation, transduction, transfection, and ultrasound transformation. Optionally, for exogenous expression in E. coli or other microbial host cells, some nucleic acid sequences in the genes or cDNAs of eukaryotic nucleic acids can encode targeting signals such as an N-terminal mitochondrial or other targeting signal, which can be removed before transformation into the prokaryotic host cells if desired. For exogenous expression in yeast or other eukaryotic host cells, genes can be expressed in the cytosol without the addition of leader sequence, or can be targeted to mitochondrion or other organelles or for secretion by the addition of a suitable targeting sequence such as a mitochondrial targeting or secretion signal suitable for the host cells. Thus, it is understood that appropriate modifications to a nucleic acid sequence to remove or include a targeting sequence, can be incorporated into an exogenous nucleic acid sequence to impart desirable properties. Furthermore, genes can be subjected to codon optimization with techniques known in the art to achieve optimized expression of the proteins.

In general, codon optimization refers to a process of modifying a nucleic acid sequence for enhanced expression in the host cell or host organism of interest by replacing at least one codon of the native sequence with codons that are more frequently or most frequently used in the genes of that host cell or host organism while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Computer algorithms for codon optimizing a particular sequence for expression in a particular host cell are available and include, e.g., Integrated DNA Technologies' Codon Optimization tool, Entelechon's Codon Usage Table Analysis Tool, GenScript's OptimumGene tool, and the like. In some embodiments, the disclosure provides codon optimized polynucleotides expressing an indigoidine synthetase described herein.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

The start of a polypeptide is known as the “N-terminus” (and also referred to as the amino-terminus, NH₂-terminus, N-terminal end or amine-terminus), which refers to the free amine (—NH₂) group of the first amino acid residue of the protein or polypeptide. The end of a polypeptide is known as the “C-terminus” (and also referred to as the carboxy-terminus, carboxyl-terminus, C-terminal end, or COOH-terminus), which refers to the free carboxyl group (—COOH) of the last amino acid residue of the protein or polypeptide. Unless otherwise specified, sequences of polypeptides throughout the present disclosure are listed from N-terminus to C-terminus, and sequences of polynucleotides throughout the present disclosure are listed from the 5′ end to the 3′ end.

An “amino acid” as used herein refers to a compound including both a carboxyl (—COOH) and amino (—NH₂) group. “Amino acid” refers to both natural and unnatural, i.e., synthetic, amino acids. Natural amino acids, with their three-letter and single-letter abbreviations, include: alanine (Ala; A); arginine (Arg, R); asparagine (Asn; N); aspartic acid (Asp; D); cysteine (Cys; C); glutamine (Gln; Q); glutamic acid (Glu; E); glycine (Gly; G); histidine (His; H); isoleucine (Ile; I); leucine (Leu; L); lysine (Lys; K); methionine (Met; M); phenylalanine (Phe; F); proline (Pro; P); serine (Ser; S); threonine (Thr; T); tryptophan (Trp; W); tyrosine (Tyr; Y); and valine (Val; V). Unnatural or synthetic amino acids include a side chain that is distinct from the natural amino acids provided above and may include, e.g., fluorophores, post-translational modifications, metal ion chelators, photocaged and photo-cross-linked moieties, uniquely reactive functional groups, and NMR, IR, and x-ray crystallographic probes. Exemplary unnatural or synthetic amino acids are provided in, e.g., Mitra et al. (2013), Mater Methods 3:204 and Wals et al. (2014), Front Chem 2:15. Unnatural amino acids may also include naturally-occurring compounds that are not typically incorporated into a protein or polypeptide, such as, e.g., citrulline (Cit), selenocysteine (Sec), and pyrrolysine (Pyl).

As used herein, the terms “non-natural,” “non-naturally occurring,” “variant,” and “mutant” are used interchangeably in the context of an organism, polypeptide, or nucleic acid. The terms “non-natural,” “non-naturally occurring,” “variant,” and “mutant” in this context refer to a polypeptide or nucleic acid sequence having at least one variation or mutation at an amino acid position or nucleic acid position as compared to a wild-type polypeptide or nucleic acid sequence. The at least one variation can be, e.g., an insertion of one or more amino acids or nucleotides, a deletion of one or more amino acids or nucleotides, or a substitution of one or more amino acids or nucleotides. A “variant” protein or polypeptide is also referred to as a “non-natural” protein or polypeptide.

Naturally-occurring organisms, nucleic acids, and polypeptides can be referred to as “wild-type” or “original” or “natural” such as wild-type strains of the referenced species, or a wild-type protein or nucleic acid sequence. Likewise, amino acids found in polypeptides of the wild type organism can be referred to as “original” or “natural” with regards to any amino acid position.

An “amino acid substitution” refers to a polypeptide that includes one or more substitutions of wild-type or naturally occurring amino acid(s) with a different amino acid relative to the wild-type or naturally occurring amino acid at that particular residue of the polypeptide. The substituted amino acid may be a synthetic or naturally occurring amino acid. In some embodiments, the substituted amino acid is a naturally occurring amino acid, e.g., Ala, Arg, Asn, Asp, Cys, Gln, Glu, Gly, His, Ile, Leu, Lys, Met, Phe, Pro, Ser, Thr, Trp, Tyr, and Val. In some embodiments, the substituted amino acid is an unnatural or synthetic amino acid. Substitution mutants may be described using an abbreviated system. For example, a substitution mutation in which the fifth (5^{th}) amino acid residue is substituted may be abbreviated as “X5Y,” wherein “X” is the wild-type or naturally occurring amino acid to be replaced, “5” is the amino acid residue position within the amino acid sequence of the protein or polypeptide, and “Y” is the substituted, or non-wild-type or non-naturally occurring, amino acid.

An “isolated” polypeptide or nucleic acid is a molecule that has been removed from its natural environment. It is also understood that “isolated” polypeptides or nucleic acids may be included in a composition, e.g., with buffers, stabilizing agents, and/or salts, and still be considered isolated. As used herein, “isolated” does not necessarily imply any particular level purity of the polypeptide or nucleic acid.

The term “recombinant” when used in reference to a nucleic acid or polypeptide means that the nucleic acid or polypeptide results from, a new combination of genetic material that is not known to exist in nature. A recombinant molecule can be produced by any of the techniques available in the field of recombinant technology, including, but not limited to, polymerase chain reaction (PCR), gene splicing (e.g., using restriction endonucleases), and solid-phase synthesis of nucleic acids and polypeptides.

The term “domain” when used in reference to a polypeptide means a distinct functional and/or structural unit in the polypeptide. Domains are sometimes responsible for a particular function or interaction, contributing to the overall role of a protein. Domains may exist in a variety of biological contexts. Similar domains may be found in proteins with different functions. Alternatively, domains with low sequence identity (i.e., less than about 50%, less than about 40%, less than about 30%, less than about 20%, less than about 10%, less than about 5%, or less than about 1% sequence identity) may have the same function.

As used herein, the term “sequence similarity” (% similarity) refers to the degree of identity or correspondence between nucleic acid or amino acid sequences. In the context of polynucleotides, “sequence similarity” may refer to nucleic acid sequences wherein changes in one or more nucleotide bases results in substitution of one or more amino acids, but do not affect the functional properties of the protein encoded by the polynucleotide. “Sequence similarity” may also refer to modifications of the polynucleotide, such as deletion or insertion of one or more nucleotide bases, that do not substantially affect the functional properties of the resulting transcript. It is therefore understood that the present disclosure encompasses more than the specific exemplary sequences. Methods of making nucleotide base substitutions are known, as are methods of determining the retention of biological activity of the polypeptide encoded by the polynucleotides.

In the context of polypeptides, “sequence similarity” refers to two or more polypeptides wherein greater than about 40% of the amino acids are identical, or greater than about 60% of the amino acids are functionally identical. “Functionally identical” or “functionally similar” amino acids have chemically similar side chains. For example, amino acids can be grouped in the following manner according to functional similarity: Positively-charged side chains: Arg, His, Lys; Negatively-charged side chains: Asp, Glu; Polar, uncharged side chains: Ser, Thr, Asn, Gln; Hydrophobic side chains: Ala, Val, Ile, Leu, Met, Phe, Tyr, Trp; Other: Cys, Gly, Pro. In some embodiments, similar polypeptides of the present disclosure have about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% functionally identical amino acids.

The “percent identity” (% identity) between two polynucleotide or amino acid sequences is determined when sequences are aligned for maximum homology, and generally not including gaps or truncations. Additional sequences added to a polypeptide, including but not limited to immunodetection tags, purification tags, localization sequences (presence or absence), etc., do not affect the % identity unless otherwise specified.

Algorithms known to those skilled in the art, such as Align, BLAST, ClustalW and others compare and determine a raw sequence similarity or identity, and also determine the presence or significance of gaps in the sequence which can be assigned a weight or score. Such algorithms also are known in the art and are similarly applicable for determining nucleotide or amino acid sequence similarity or identity, and can be useful in identifying orthologs of genes of interest.

In some embodiments, similar polynucleotides of the present disclosure have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical nucleotide sequences.

In some embodiments, similar polypeptides of the present disclosure have about 40%, at least about 40%, about 45%, at least about 45%, about 50%, at least about 50%, about 55%, at least about 55%, about 60%, at least about 60%, about 65%, at least about 65%, about 70%, at least about 70%, about 75%, at least about 75%, about 80%, at least about 80%, about 85%, at least about 85%, about 90%, at least about 90%, about 95%, at least about 95%, about 97%, at least about 97%, about 98%, at least about 98%, about 99%, at least about 99%, or about 100% identical amino acid sequences.

A homolog is a gene or genes that are related by vertical descent and are responsible for substantially the same or identical functions in different organisms. Genes are related by vertical descent when, for example, they share sequence similarity of sufficient amount to indicate they are homologous or related by evolution from a common ancestor. Genes can also be considered orthologs if they share three-dimensional structure but not necessarily sequence similarity, of a sufficient amount to indicate that they have evolved from a common ancestor to the extent that the primary sequence similarity is not identifiable. Paralogs are genes related by duplication within a genome, and can evolve new functions, even if these are related to the original one.

An amino acid position (or simply, amino acid) “corresponding to” an amino acid position in another polypeptide sequence is the position that is aligned with the referenced amino acid position when the polypeptides are aligned for maximum homology, for example, as determined by BLAST, which allows for gaps in sequence homology within protein sequences to align related sequences and domains. Alternatively, in some instances, when polypeptide sequences are aligned for maximum homology, a corresponding amino acid may be the nearest amino acid to the identified amino acid that is within the same amino acid biochemical grouping—i.e., the nearest acidic amino acid, the nearest basic amino acid, the nearest aromatic amino acid, etc. to the identified amino acid.

By “substantially identical,” with reference to a nucleic acid sequence (e.g., a gene, RNA, or cDNA) or amino acid sequence (e.g., a protein or polypeptide) is meant one that has at least at least 80%, at least 85%, at least 90%, at least 95%, at least 96%, at least 97% at least 98%, or at least 99% nucleotide or amino acid identity, respectively, to a reference sequence.

As used in the context of proteins, the term “structural similarity” indicates the degree of homology between the overall shape, fold, and/or topology of the proteins. It should be understood that two proteins do not necessarily need to have high sequence similarity to achieve structural similarity. Protein structural similarity is often measured by root mean squared deviation (RMSD), global distance test score (GDT-score), and template modeling score (TM-score); see, e.g., Xu and Zhang (2010), Bioinformatics 26(7):889-895. Structural similarity can be determined, e.g., by superimposing protein structures obtained from, e.g., x-ray crystallography, NMR spectroscopy, cryogenic electron microscopy (cryo-EM), mass spectrometry, or any combination thereof, and calculating the RMSD, GDT-score, and/or TM-score based on the superimposed structures. In some embodiments, two proteins have substantially similar tertiary structures when the TM-score is greater than about 0.5, greater than about 0.6, greater than about 0.7, greater than about 0.8, or greater than about 0.9. In some embodiments, two proteins have substantially identical tertiary structures when the TM-score is about 1.0. Structurally-similar proteins may also be identified computationally using algorithms such as, e.g., TM-align, DALI, STRUCTAL, MINRMS, and the like.

### Indigoidine Biosynthesis

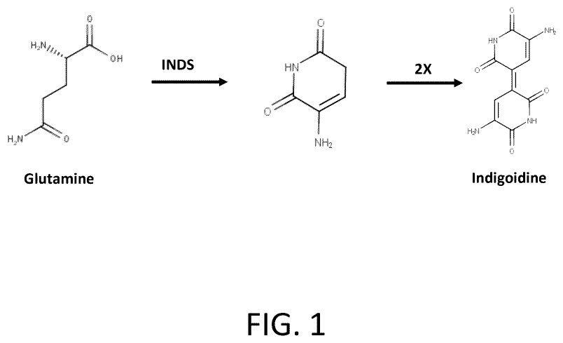

Biosynthesis of indigoidine involves the condensation of two units of L-glutamine by a non-ribosomal peptide synthetase (NRPS) referred to herein as indigoidine synthetase (INDS). See, e.g. FIG. 1. In general, INDS includes adenylation (A), oxidation (Ox), thiolation (T) (also known as the peptidyl carrier protein or PCP), and thioesterase (TE) domains. Bioproduction of indigoidine using INDS derived from Streptomyces lavendulae, Streptomyces chromofuscus, and/or Erwinia chrysanthemi in heterologous hosts, including E. coli, Saccharomyces cerevisiae, Rhodosporidium toruloides, Pseudomonas putida, Vibrio natriegens, Corynebacterium glutamicum, roses, and silkworm glands, has been reported. See, e.g., U.S. Pat. No. 9,243,269; Wehrs et al., Green Chem. (2019) 21:3394-3406; and Ghiffary et al., ACS Sustainable Chem. Eng. (2021) 9:6613-6622.

The present inventors have discovered alternative INDSs that provide improved heterologous bioproduction of indigoidine. In some embodiments, the INDS is derived from Archangium violaceum, Photorhabdus luminescens, Clavibacter michiganensis subsp. insidiosus, Vibrio spartinae, Dickeya dadantii, or Arthrobacter antioxidans. In some embodiments, INDS comprises a polypeptide sequence having at least 50%, at least 60%, at least 70%, at least 75%, at least 80%, at least 85%, at least 90%, at least 91%, at least 92%, at least 93%, at least 94%, at least 95%, at least 96%, at last 97%, at least 98%, at least 99%, or 100% sequence identity to any one of SEQ ID NOs: 1-7. In some embodiments, INDS comprises a polypeptide sequence having at least 50%, at least 60%, at least 70%, at least 75%, at least 80%, at least 85%, at least 90%, at least 91%, at least 92%, at least 93%, at least 94%, at least 95%, at least 96%, at last 97%, at least 98%, at least 99%, or 100% sequence identity to any one of SEQ ID NOs: 1-5. In some embodiments, the INDS is capable of producing indigoidine from glutamine, e.g., via the biosynthesis pathway shown in FIG. 1. In some embodiments, the INDS is capable of producing indigoidine in the heterologous host.

In some embodiments, the present disclosure provides a heterologous host capable of expressing an INDS comprising at least 70% sequence identity to any one of SEQ ID NOs: 1-5, wherein the heterologous host cell is capable of producing indigoidine. In some embodiments, the present disclosure provides a heterologous expression system for the production of indigoidine, wherein the expression system comprises a heterologous host cell comprising a heterologous nucleic acid, wherein the heterologous nucleic acid encodes an INDS described herein, e.g., comprising at least 70% sequence identity to any one of SEQ ID NOs: 1-5. In some embodiments, the INDS is expressed in the heterologous host cell such that the heterologous host cell is capable of producing indigoidine, e.g., from glutamine as described herein. In some embodiments, the heterologous host cell does not comprise an endogenous INDS. In some embodiments, the heterologous host cell is only capable of producing indigoidine upon expression of the INDS described herein.

As described herein, a “heterologous host cell” refers to a cell of an organism that is not the same species as the organism from which the INDS is derived, i.e., the INDS is “heterologous” to the host cell. In some embodiments, the host cell is not any of Archangium violaceum, Photorhabdus luminescens, Clavibacter michiganensis subsp. insidiosus, Vibrio spartinae, or Arthrobacter antioxidans. In some embodiments, the host cell can be any one of Archangium violaceum, Photorhabdus luminescens, Clavibacter michiganensis subsp. insidiosus, Vibrio spartinae, or Arthrobacter antioxidans, provided that the INDS introduced into the host cell is not derived from the same species. Host cells are further described herein.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 1. In some embodiments, the INDS comprises SEQ ID NO:1. SEQ ID NO:1 describes the amino acid sequence of an amino acid adenylation domain-containing protein from Archangium violaceum. In embodiments where the INDS comprises SEQ ID NO:1, the heterologous host cell is not Archangium violaceum. In some embodiments, the INDS is capable of producing indigoidine, e.g., from glutamine as described herein.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:2. In some embodiments, the INDS comprises SEQ ID NO:2. SEQ ID NO:2 describes the amino acid sequence of an amino acid adenylation domain-containing protein from Photorhabdus luminescens. In embodiments where the INDS comprises SEQ ID NO:2, the heterologous host cell is not Photorhabdus luminescens. In some embodiments, the INDS is capable of producing indigoidine, e.g., from glutamine as described herein.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:3. In some embodiments, the INDS comprises SEQ ID NO:3. SEQ ID NO:3 describes the amino acid sequence of an amino acid adenylation domain-containing protein from Clavibacter michiganensis subsp. insidiosus. In embodiments where the INDS comprises SEQ ID NO:3, the heterologous host cell is not Clavibacter michiganensis subsp. insidiosus. In some embodiments, the INDS is capable of producing indigoidine, e.g., from glutamine as described herein.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:4. In some embodiments, the INDS comprises SEQ ID NO:4. SEQ ID NO:4 describes the amino acid sequence of a tyrocidine synthase from Vibrio spartinae. In embodiments where the INDS comprises SEQ ID NO: 4, the heterologous host cell is not Vibrio spartinae. In some embodiments, the INDS is capable of producing indigoidine, e.g., from glutamine as described herein.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:5. In some embodiments, the INDS comprises SEQ ID NO:5. SEQ ID NO:5 describes the amino acid sequence of an amino acid adenylation domain-containing protein from Arthrobacter antioxidans. In embodiments where the INDS comprises SEQ ID NO:5, the heterologous host cell is not Arthrobacter antioxidans. In some embodiments, the INDS is capable of producing indigoidine, e.g., from glutamine as described herein.

In some embodiments, the INDS comprises at least 70%, at least 75%, at least 80%, at least 85%, at least 90%, at least 91%, at least 92%, at least 93%, at least 94%, or about 95% sequence identity to SEQ ID NO:6. SEQ ID NO:6 describes the amino acid sequence of a putative indigoidine synthase from Dickeya dadantii. In some embodiments, the INDS comprises at least 70% to about 95% sequence identity to SEQ ID NO:6 and the heterologous host cell is not Dickeya dadantii. In some embodiments, the INDS is capable of producing indigoidine, e.g., from glutamine as described herein.

In some embodiments, the INDS comprises at least 70%, at least 75%, at least 80%, at least 85%, at least 90%, at least 91%, at least 92%, at least 93%, at least 94%, or about 95% sequence identity to SEQ ID NO:7. SEQ ID NO:7 describes the amino acid sequence of a putative indigoidine synthase from Photorhabdus laumondii. In some embodiments, the INDS comprises at least 70% to about 95% sequence identity to SEQ ID NO:7 and the heterologous host cell is not Photorhabdus laumondii. In some embodiments, the INDS is capable of producing indigoidine, e.g., from glutamine as described herein.

In some embodiments, the INDS comprises at least 70%, at least 75%, at least 80%, at least 85%, at least 90%, at least 91%, at least 92%, at least 93%, at least 94%, or about 95% sequence identity to SEQ ID NO:8. SEQ ID NO:8 describes the amino acid sequence of a putative indigoidine synthase from Vogesella indigofera. In some embodiments, the INDS comprises at least 70% to about 95% sequence identity to SEQ ID NO:8 and the heterologous host cell is not Vogesella indigofera. In some embodiments, the INDS is capable of producing indigoidine, e.g., from glutamine as described herein.

### Phosphopantetheinyl Transferase

In some embodiments, the heterologous host cell is capable of expressing an INDS as described herein, and is further capable of expressing a 4′-phosphopantetheinyl transferase (PPTase). In general, PPTases are responsible for phosphospantetheinylation of enzymes such as fatty acid synthases (FAS), polyketide synthases (PKS), and non-ribosomal peptide synthetases (NRPS). The phosphopantetheinylation reaction is critical for enzyme activation and involves transfer of a PPT moiety of coenzyme A (CoA) to the carrier domain of the enzyme. PPTases are described, e.g., in Beld et al., Nat. Prod. Rep. (2014) 31:61-108.

In some embodiments, the INDS is activated via phosphopantetheinylation. As referred to herein, an “activated” INDS (also known as “holo” INDS) includes a 4′-phosphopantetheine (PPT) group in its carrier domain and catalyzes the conversion of glutamine to indigoidine. In contrast, an “inactive” INDS (also known as “apo” INDS) does not include the PPT group and does not convert glutamine to indigoidine. In some embodiments, the INDS is phosphopantetheinylated by a PPTase. PPTases are generally found in all organisms; however, not all PPTases are capable of phosphopanthetheinylating, and therefore activating, the INDSs described herein. In some embodiments, the PPTase expressed by the heterologous host cell is capable of phosphospantetheinylating, and therefore activating, the INDS expressed by the heterologous host cell.

In some embodiments, the PPTase expressed by the heterologous host cell is an endogenous PPTase of the host cell. In some embodiments, the endogenous PPTase of the host cell is capable of activating the INDS provided herein. For example, various Bacillus, Corynebacterium, Streptomyces, and Pseudomonas species include endogenous PPTases that are capable of activating the INDSs provided herein. In some embodiments, the heterologous host cell capable of expressing the INDS described herein is a Bacillus, Corynebacterium, Streptomyces, or Pseudomonas cell. In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5.

In some embodiments, the PPTase expressed by the heterologous host cell is an exogenous PPTase. In some embodiments, the exogenous PPTase is capable of activating the INDS expressed by the host cell. It will be understood by one of ordinary skill in the art that “exogenous” PPTase encompasses both a non-native PPTase being introduced to the cell as well as overexpression of a native PPTase at a level such that it is capable of activating the INDS. For example, the E. coli endogenous PPTase entD may not be capable of activating INDS at its native expression levels, but may be capable of activating INDS when overexpressed.

In some embodiments, the exogenous PPTase is from Dickeya chrysanthemi, Photorhabdus laumondii, Pseudomonas putida, Bacillus subtilis, Clavibacter michiganensis, Streptomyces albidoflavus, Streptomyces avermitilis, Streptomyces clavuligerus, Streptomyces lividans, Streptomyces venezuelae, or Vogesella indigofera. In some embodiments, the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21. In some embodiments, the PPTase comprises any one of SEQ ID NOs: 9-21.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5, and the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:9. In some embodiments, the PPTase is capable of activating the INDS.

In embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5, and the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:9 and comprises a mutation at amino acid position 198 of SEQ ID NO:9. In embodiments, the mutation of SEQ ID NO:9 comprises an Asp to Gly substitution (D198G). In some embodiments, the PPTase is capable of activating the INDS.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5, and the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 10. In some embodiments, the PPTase is capable of activating the INDS.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5, and the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 11. In some embodiments, the PPTase is capable of activating the INDS.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5, and the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:12. In some embodiments, the PPTase is capable of activating the INDS.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5, and the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:13. In some embodiments, the PPTase is capable of activating the INDS.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5, and the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 14. In some embodiments, the PPTase is capable of activating the INDS.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5, and the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 15. In some embodiments, the PPTase is capable of activating the INDS.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5, and the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 16. In some embodiments, the PPTase is capable of activating the INDS.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5, and the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:17. In some embodiments, the PPTase is capable of activating the INDS.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5, and the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 18. In some embodiments, the PPTase is capable of activating the INDS.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5, and the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 19. In some embodiments, the PPTase is capable of activating the INDS.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5, and the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:20. In some embodiments, the PPTase is capable of activating the INDS.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5, and the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:21. In some embodiments, the PPTase is capable of activating the INDS.

In some embodiments, the heterologous host cell is capable of expressing an INDS and a PPTase according to any of Combinations (1A)-(65) in Table 1. In Table 1, “9+D198G” refers to a PPTase comprising SEQ ID NO:9 and comprising a Asp to Gly mutation at amino acid position 198 of SEQ ID NO:9.

TABLE 1
Combinations of INDS and PPTase

| Combo. # | INDS SEQ ID NO: | PPTase SEQ ID NO: |
|---|---|---|
| (1A) | 1 | 9 |
| (1B) | 1 | 9 + D198G |
| (2) | 1 | 10 |
| (3) | 1 | 11 |
| (4) | 1 | 12 |
| (5) | 1 | 13 |
| (6) | 1 | 14 |
| (7) | 1 | 15 |
| (8) | 1 | 16 |
| (9) | 1 | 17 |
| (10) | 1 | 18 |
| (11) | 1 | 19 |
| (12) | 1 | 20 |
| (13) | 1 | 21 |
| (14A) | 2 | 9 |
| (14B) | 2 | 9 + D198G |
| (15) | 2 | 10 |
| (16) | 2 | 11 |
| (17) | 2 | 12 |
| (18) | 2 | 13 |
| (19) | 2 | 14 |
| (20) | 2 | 15 |
| (21) | 2 | 16 |
| (22) | 2 | 17 |
| (23) | 2 | 18 |
| (24) | 2 | 19 |
| (25) | 2 | 20 |
| (26) | 2 | 21 |
| (27A) | 3 | 9 |
| (27B) | 3 | 9 + D198G |
| (28) | 3 | 10 |
| (29) | 3 | 11 |
| (30) | 3 | 12 |
| (31) | 3 | 13 |
| (32) | 3 | 14 |
| (33) | 3 | 15 |
| (34) | 3 | 16 |
| (35) | 3 | 17 |
| (36) | 3 | 18 |
| (37) | 3 | 19 |
| (38) | 3 | 20 |
| (39) | 3 | 21 |
| (40A) | 4 | 9 |
| (40B) | 4 | 9 + D198G |
| (41) | 4 | 10 |
| (42) | 4 | 11 |
| (43) | 4 | 12 |
| (44) | 4 | 13 |
| (45) | 4 | 14 |
| (46) | 4 | 15 |
| (47) | 4 | 16 |
| (48) | 4 | 17 |
| (49) | 4 | 18 |
| (50) | 4 | 19 |
| (51) | 4 | 20 |
| (52) | 4 | 21 |
| (53A) | 5 | 9 |
| (53B) | 5 | 9 + D198G |
| (54) | 5 | 10 |
| (55) | 5 | 11 |
| (56) | 5 | 12 |
| (57) | 5 | 13 |
| (58) | 5 | 14 |
| (59) | 5 | 15 |
| (60) | 5 | 16 |
| (61) | 5 | 17 |
| (62) | 5 | 18 |
| (63) | 5 | 19 |
| (64) | 5 | 20 |
| (65) | 5 | 21 |

In some embodiments, the INDS and the PPTase each independently comprises at least 75%, at least 80%, at least 85%, at least 90%, at least 91%, at least 92%, at least 93%, at least 94%, at least 95%, at least 96%, at least 97%, at least 98%, at least 99%, or 100% sequence identity to the sequences indicated in each combination of Table 1. In some embodiments, the INDS and the PPTase each comprises at least 90% sequence identity to the sequences indicated in each combination of Table 1. In some embodiments, the INDS comprises at least 90% sequence identity to SEQ ID NO:1, and the PPTase comprises at least 90% sequence identity to SEQ ID NO:9. In some embodiments, the INDS comprises at least 90% sequence identity to SEQ ID NO:1, and the PPTase comprises at least 90% sequence identity to SEQ ID NO:9 with a D198G mutation.

### NRPS Accessory Protein

In some embodiments, the heterologous host cell, which is capable of expressing the INDS and/or the PPTase as described herein, is further capable of expressing a NRPS accessory protein. In some embodiments, the NRPS accessory protein improves the solubility and/or function of the INDS. In some embodiments, the NRPS accessory protein is an MbtH-like protein (MLP). In general, MLPs are small proteins of approximately 70 amino acids and have been shown to influence solubility, substrate affinity, and enzyme turnover, both positively and negatively. See, e.g., Esquilín-Lebrón et al., J. Bacteriol. (2018) 100:10.1128/jb.00346-18. For example, it was demonstrated that non-optimal of MLP and NRPS can result in decreased reaction rate and enzyme turnover, and that incorrect MLP/NRPS pairings can be detrimental to NRPS catalysis. Schomer and Thomas, Biochemistry (2017) 56:5380-5390.

In some embodiments, the MLP expressed by the heterologous host cell is an endogenous MLP of the host cell. In some embodiments, the endogenous MLP of the host cell is capable of improving the solubility of the INDS provided herein. In some embodiments, the MLP expressed by the heterologous host cell is an exogenous MLP. In some embodiments, the MLP is a native MLP of the host cell that is overexpressed at a level such that it is capable of improving solubility of the INDS.

In some embodiments, the MLP is from Streptomyces lavendulae, Archangium violaceum, or Myxococcus. In some embodiments, the MLP comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:22, SEQ ID NO: 23, or SEQ ID NO:24. In some embodiments, the MLP comprises SEQ ID NO:22, SEQ ID NO: 23, or SEQ ID NO:24.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5; the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21; and the MLP comprises SEQ ID NO: 22.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5; the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21; and the MLP comprises SEQ ID NO: 23.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5; the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21; and the MLP comprises SEQ ID NO: 24.

In some embodiments, the heterologous host cell is capable of expressing an INDS, a PPTase, and an MLP according to any of Combinations (1A)-(195) in Table 2. In Table 2, “9+D198G” refers to a PPTase comprising SEQ ID NO:9 and comprising an Asp to Gly mutation at amino acid position 198 of SEQ ID NO:9.

TABLE 2
Combinations of INDS, PPTase, and MLP

| # | I* | P** | M*** |
|---|---|---|---|
| (1A) | 1 | 9 | 22 |
| (1B) | 1 | 9 + D198G | 22 |
| (2) | 1 | 10 | 22 |
| (3) | 1 | 11 | 22 |
| (4) | 1 | 12 | 22 |
| (5) | 1 | 13 | 22 |
| (6) | 1 | 14 | 22 |
| (7) | 1 | 15 | 22 |
| (8) | 1 | 16 | 22 |
| (9) | 1 | 17 | 22 |
| (10) | 1 | 18 | 22 |
| (11) | 1 | 19 | 22 |
| (12) | 1 | 20 | 22 |
| (13) | 1 | 21 | 22 |
| (14A) | 2 | 9 | 22 |
| (14B) | 2 | 9 + D198G | 22 |
| (15) | 2 | 10 | 22 |
| (16) | 2 | 11 | 22 |
| (17) | 2 | 12 | 22 |
| (18) | 2 | 13 | 22 |
| (19) | 2 | 14 | 22 |
| (20) | 2 | 15 | 22 |
| (21) | 2 | 16 | 22 |
| (22) | 2 | 17 | 22 |
| (23) | 2 | 18 | 22 |
| (24) | 2 | 19 | 22 |
| (25) | 2 | 20 | 22 |
| (26) | 2 | 21 | 22 |
| (27A) | 3 | 9 | 22 |
| (27B) | 3 | 9 + D198G | 22 |
| (28) | 3 | 10 | 22 |
| (29) | 3 | 11 | 22 |
| (30) | 3 | 12 | 22 |
| (31) | 3 | 13 | 22 |
| (32) | 3 | 14 | 22 |
| (33) | 3 | 15 | 22 |
| (34) | 3 | 16 | 22 |
| (35) | 3 | 17 | 22 |
| (36) | 3 | 18 | 22 |
| (37) | 3 | 19 | 22 |
| (38) | 3 | 20 | 22 |
| (39) | 3 | 21 | 22 |
| (40A) | 4 | 9 | 22 |
| (40B) | 4 | 9 + D198G | 22 |
| (41) | 4 | 10 | 22 |
| (42) | 4 | 11 | 22 |
| (43) | 4 | 12 | 22 |
| (44) | 4 | 13 | 22 |
| (45) | 4 | 14 | 22 |
| (46) | 4 | 15 | 22 |
| (47) | 4 | 16 | 22 |
| (48) | 4 | 17 | 22 |
| (49) | 4 | 18 | 22 |
| (50) | 4 | 19 | 22 |
| (51) | 4 | 20 | 22 |
| (52) | 4 | 21 | 22 |
| (53A) | 5 | 9 | 22 |
| (53B) | 5 | 9 + D198G | 22 |
| (54) | 5 | 10 | 22 |
| (55) | 5 | 11 | 22 |
| (56) | 5 | 12 | 22 |
| (57) | 5 | 13 | 22 |
| (58) | 5 | 14 | 22 |
| (59) | 5 | 15 | 22 |
| (60) | 5 | 16 | 22 |
| (61) | 5 | 17 | 22 |
| (62) | 5 | 18 | 22 |
| (63) | 5 | 19 | 22 |
| (64) | 5 | 20 | 22 |
| (65) | 5 | 21 | 22 |
| (66A) | 1 | 9 | 23 |
| (66B) | 1 | 9 + D198G | 23 |
| (67) | 1 | 10 | 23 |
| (68) | 1 | 11 | 23 |
| (69) | 1 | 12 | 23 |
| (70) | 1 | 13 | 23 |
| (71) | 1 | 14 | 23 |
| (72) | 1 | 15 | 23 |
| (73) | 1 | 16 | 23 |
| (74) | 1 | 17 | 23 |
| (75) | 1 | 18 | 23 |
| (76) | 1 | 19 | 23 |
| (77) | 1 | 20 | 23 |
| (78) | 1 | 21 | 23 |
| (79A) | 2 | 9 | 23 |
| (79B) | 2 | 9 + D198G | 23 |
| (80) | 2 | 10 | 23 |
| (81) | 2 | 11 | 23 |
| (82) | 2 | 12 | 23 |
| (83) | 2 | 13 | 23 |
| (84) | 2 | 14 | 23 |
| (85) | 2 | 15 | 23 |
| (86) | 2 | 16 | 23 |
| (87) | 2 | 17 | 23 |
| (88) | 2 | 18 | 23 |
| (89) | 2 | 19 | 23 |
| (90) | 2 | 20 | 23 |
| (91) | 2 | 21 | 23 |
| (92A) | 3 | 9 | 23 |
| (92B) | 1 | 9 + D198G | 23 |
| (93) | 3 | 10 | 23 |
| (94) | 3 | 11 | 23 |
| (95) | 3 | 12 | 23 |
| (96) | 3 | 13 | 23 |
| (97) | 3 | 14 | 23 |
| (98) | 3 | 15 | 23 |
| (99) | 3 | 16 | 23 |
| (100) | 3 | 17 | 23 |
| (101) | 3 | 18 | 23 |
| (102) | 3 | 19 | 23 |
| (103) | 3 | 20 | 23 |
| (104) | 3 | 21 | 23 |
| (105A) | 4 | 9 | 23 |
| (105B) | 4 | 9 + D198G | 23 |
| (106) | 4 | 10 | 23 |
| (107) | 4 | 11 | 23 |
| (108) | 4 | 12 | 23 |
| (109) | 4 | 13 | 23 |
| (110) | 4 | 14 | 23 |
| (111) | 4 | 15 | 23 |
| (112) | 4 | 16 | 23 |
| (113) | 4 | 17 | 23 |
| (114) | 4 | 18 | 23 |
| (115) | 4 | 19 | 23 |
| (116) | 4 | 20 | 23 |
| (117) | 4 | 21 | 23 |
| (118A) | 5 | 9 | 23 |
| (118B) | 5 | 9 + D198G | 23 |
| (119) | 5 | 10 | 23 |
| (120) | 5 | 11 | 23 |
| (121) | 5 | 12 | 23 |
| (122) | 5 | 13 | 23 |
| (123) | 5 | 14 | 23 |
| (124) | 5 | 15 | 23 |
| (125) | 5 | 16 | 23 |
| (126) | 5 | 17 | 23 |
| (127) | 5 | 18 | 23 |
| (128) | 5 | 19 | 23 |
| (129) | 5 | 20 | 23 |
| (130) | 5 | 21 | 23 |
| (131A) | 1 | 9 | 24 |
| (131B) | 1 | 9 + D198G | 24 |
| (132) | 1 | 10 | 24 |
| (133) | 1 | 11 | 24 |
| (134) | 1 | 12 | 24 |
| (135) | 1 | 13 | 24 |
| (136) | 1 | 14 | 24 |
| (137) | 1 | 15 | 24 |
| (138) | 1 | 16 | 24 |
| (139) | 1 | 17 | 24 |
| (140) | 1 | 18 | 24 |
| (141) | 1 | 19 | 24 |
| (142) | 1 | 20 | 24 |
| (143) | 1 | 21 | 24 |
| (144A) | 2 | 9 | 24 |
| (144B) | 2 | 9 + D198G | 24 |
| (145) | 2 | 10 | 24 |
| (146) | 2 | 11 | 24 |
| (147) | 2 | 12 | 24 |
| (148) | 2 | 13 | 24 |
| (149) | 2 | 14 | 24 |
| (150) | 2 | 15 | 24 |
| (151) | 2 | 16 | 24 |
| (152) | 2 | 17 | 24 |
| (153) | 2 | 18 | 24 |
| (154) | 2 | 19 | 24 |
| (155) | 2 | 20 | 24 |
| (156) | 2 | 21 | 24 |
| (157A) | 3 | 9 | 24 |
| (157B) | 1 | 9 + D198G | 24 |
| (158) | 3 | 10 | 24 |
| (159) | 3 | 11 | 24 |
| (160) | 3 | 12 | 24 |
| (161) | 3 | 13 | 24 |
| (162) | 3 | 14 | 24 |
| (163) | 3 | 15 | 24 |
| (164) | 3 | 16 | 24 |
| (165) | 3 | 17 | 24 |
| (166) | 3 | 18 | 24 |
| (167) | 3 | 19 | 24 |
| (168) | 3 | 20 | 24 |
| (169) | 3 | 21 | 24 |
| (170A) | 4 | 9 | 24 |
| (170B) | 4 | 9 + D198G | 24 |
| (171) | 4 | 10 | 24 |
| (172) | 4 | 11 | 24 |
| (173) | 4 | 12 | 24 |
| (174) | 4 | 13 | 24 |
| (175) | 4 | 14 | 24 |
| (176) | 4 | 15 | 24 |
| (177) | 4 | 16 | 24 |
| (178) | 4 | 17 | 24 |
| (179) | 4 | 18 | 24 |
| (180) | 4 | 19 | 24 |
| (181) | 4 | 20 | 24 |
| (182) | 4 | 21 | 24 |
| (183A) | 5 | 9 | 24 |
| (183B) | 5 | 9 + D198G | 24 |
| (184) | 5 | 10 | 24 |
| (185) | 5 | 11 | 24 |
| (186) | 5 | 12 | 24 |
| (187) | 5 | 13 | 24 |
| (188) | 5 | 14 | 24 |
| (189) | 5 | 15 | 24 |
| (190) | 5 | 16 | 24 |
| (191) | 5 | 17 | 24 |
| (192) | 5 | 18 | 24 |
| (193) | 5 | 19 | 24 |
| (194) | 5 | 20 | 24 |
| (195) | 5 | 21 | 24 |

# = Combination #;
I* = INDS SEQ ID NO;
P** = PPTase SEQ ID NO;
M*** = MLP SEQ ID NO

In some embodiments, the INDS, PPTase, and MLP each independently comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the sequences indicated in each combination of Table 2. In some embodiments, the INDS, PPTase, and MLP each comprises at least 90% sequence identity to the sequences indicated in each combination of Table 2. In some embodiments, the INDS comprises at least 90% sequence identity to SEQ ID NO: 1, the PPTase comprises at least 90% sequence identity to SEQ ID NO:9 optionally comprising a D198G mutation, and the MLP comprises at least 90% sequence identity to SEQ ID NO: 22. In some embodiments, the INDS comprises at least 90% sequence identity to SEQ ID NO:1, the PPTase comprises at least 90% sequence identity to SEQ ID NO:9 optionally comprising a D198G mutation, and the MLP comprises at least 90% sequence identity to SEQ ID NO:23. In some embodiments, the INDS comprises at least 90% sequence identity to SEQ ID NO:1, the PPTase comprises at least 90% sequence identity to SEQ ID NO:9 optionally comprising a D198G mutation, and the MLP comprises at least 90% sequence identity to SEQ ID NO:24.

### Indigoidine Transporter Protein

In some embodiments, the heterologous host cell, which is capable of expressing the INDS, the PPTase, and/or the NRPS accessory protein as described herein, is further capable of expressing an indigoidine transporter protein. In some embodiments, the indigoidine transporter protein improves the function of the INDS. In some embodiments, the indigoidine transporter protein improves titer of the indigoidine produced in the heterologous host cell. In some embodiments, the indigoidine transporter protein improves growth of the heterologous host cell.

In some embodiments, the indigoidine transporter protein is from Vogesella indigofera, Dickeya dadantii (e.g., strain 3937), Archangium violaceum, or Photorhabdus laumondii (e.g., subsp. laumondii TTO1). In some embodiments, the indigoidine transporter protein is a drug/metabolite transporter (DMT) family transporter. In some embodiments, the indigoidine transporter protein is a mitochondrial folate transporter (MTF) family transporter. In some embodiments, the indigoidine transporter protein comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 25-32.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5; the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21; the MLP comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 22-24; and the indigoidine transporter protein comprises SEQ ID NO:25, or the indigoidine transporter protein comprises a protein of SEQ ID NO:25 and one or more additional proteins independently selected from SEQ ID NOs: 26-32.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5; the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21; the MLP comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 22-24; and the indigoidine transporter protein comprises SEQ ID NO:26, or the indigoidine transporter protein comprises a protein of SEQ ID NO:26 and one or more additional proteins independently selected from SEQ ID NOs: 25 and 27-32.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5; the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21; the MLP comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 22-24; and the indigoidine transporter protein comprises SEQ ID NO:27, or the indigoidine transporter protein comprises a protein of SEQ ID NO:27 and one or more additional proteins independently selected from SEQ ID NOs: 25-26 and 28-32.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5; the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21; the MLP comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 22-24; and the indigoidine transporter protein comprises SEQ ID NO:28, or the indigoidine transporter protein comprises a protein of SEQ ID NO:28 and one or more additional proteins independently selected from SEQ ID NOs: 25-27 and 29-32.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5; the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21; the MLP comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 22-24; and the indigoidine transporter protein comprises SEQ ID NO:29, or the indigoidine transporter protein comprises a protein of SEQ ID NO:29 and one or more additional proteins independently selected from SEQ ID NOs: 25-28 and 30-32.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5; the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21; the MLP comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 22-24; and the indigoidine transporter protein comprises SEQ ID NO:30, or the indigoidine transporter protein comprises a protein of SEQ ID NO:30 and one or more additional proteins independently selected from SEQ ID NOs: 25-29 and 31-32.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5; the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21; the MLP comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 22-24; and the indigoidine transporter protein comprises SEQ ID NO:31, or the indigoidine transporter protein comprises a protein of SEQ ID NO:27 and one or more additional proteins independently selected from SEQ ID NOs: 25-30 and 32.

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5; the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21; the MLP comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 22-24; and the indigoidine transporter protein comprises SEQ ID NO:32, or the indigoidine transporter protein comprises a protein of SEQ ID NO:27 and one or more additional proteins independently selected from SEQ ID NOs: 25-31.

In some embodiments, the heterologous host cell is capable of expressing an INDS, a PPTase, and an MLP according to any of Combinations (1A)-(195) in Table 2, and is further capable of expressing an indigoidine transporter protein of SEQ ID NO:25. In some embodiments, the heterologous host cell is capable of expressing an INDS, a PPTase, and an MLP according to any of Combinations (1A)-(195) in Table 2, and is further capable of expressing an indigoidine transporter protein of SEQ ID NO:26. In some embodiments, the heterologous host cell is capable of expressing an INDS, a PPTase, and an MLP according to any of Combinations (1A)-(195) in Table 2, and is further capable of expressing an indigoidine transporter protein of SEQ ID NO:27. In some embodiments, the heterologous host cell is capable of expressing an INDS, a PPTase, and an MLP according to any of Combinations (1A)-(195) in Table 2, and is further capable of expressing an indigoidine transporter protein of SEQ ID NO:28. In some embodiments, the heterologous host cell is capable of expressing an INDS, a PPTase, and an MLP according to any of Combinations (1A)-(195) in Table 2, and is further capable of expressing an indigoidine transporter protein of SEQ ID NO:29. In some embodiments, the heterologous host cell is capable of expressing an INDS, a PPTase, and an MLP according to any of Combinations (1A)-(195) in Table 2, and is further capable of expressing an indigoidine transporter protein of SEQ ID NO:30. In some embodiments, the heterologous host cell is capable of expressing an INDS, a PPTase, and an MLP according to any of Combinations (1A)-(195) in Table 2, and is further capable of expressing an indigoidine transporter protein of SEQ ID NO:31. In some embodiments, the heterologous host cell is capable of expressing an INDS, a PPTase, and an MLP according to any of Combinations (1A)-(195) in Table 2, and is further capable of expressing an indigoidine transporter protein of SEQ ID NO:32.

In some embodiments, the heterologous host cell is capable of expressing at least two indigoidine transporter proteins selected from SEQ ID NOs: 25-32. In some embodiments, the heterologous host cell is capable of expressing (1) a first indigoidine transporter protein comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:25; (2) a second indigoidine transporter protein comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:26; (3) a third indigoidine transporter protein comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 27; (4) a fourth indigoidine transporter protein comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:28; (5) a fifth indigoidine transporter protein comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:29; (6) a sixth indigoidine transporter protein comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:30; (7) a seventh indigoidine transporter protein comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:31; (8) an eighth indigoidine transporter protein comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:32 or (9) any combination of (1)-(8). In some embodiments, the heterologous host cell expresses any combination of (1)-(8) as described above according to Table 3.

TABLE 3
Combination of Indigoidine Transporter Proteins

| | | | | | |
|---|---|---|---|---|---|
| (1), (2) | (1), (3) | (1), (4) | (1), (5) | (1), (6) | (1), (7) |
| (1), (8) | (2), (3) | (2), (4) | (2), (5) | (2), (6) | (2), (7) |
| (2), (8) | (3), (4) | (3), (5) | (3), (6) | (3), (7) | (3), (8) |
| (4), (5) | (4), (6) | (4), (7) | (4), (8) | (5), (6) | (5), (7) |
| (5), (8) | (6), (7) | (6), (8) | (7), (8) | (1), (2), (3) | (1), (2), (4) |
| (1), (2), (5) | (1), (2), (6) | (1), (2), (7) | (1), (2), (8) | (1), (3), (4) | (1), (3), (5) |
| (1), (3), (6) | (1), (3), (7) | (1), (3), (8) | (1), (4), (5) | (1), (4), (6) | (1), (4), (7) |
| (1), (4), (8) | (1), (5), (6) | (1), (5), (7) | (1), (5), (8) | (1), (6), (7) | (1), (6), (8) |
| (1), (7), (8) | (2), (3), (4) | (2), (3), (5) | (2), (3), (6) | (2), (3), (7) | (2), (3), (8) |
| (2), (4), (5) | (2), (4), (6) | (2), (4), (7) | (2), (4), (8) | (2), (5), (6) | (2), (5), (7) |
| (2), (5), (8) | (2), (6), (7) | (2), (6), (8) | (2), (7), (8) | (3), (4), (5) | (3), (4), (6) |
| (3), (4), (7) | (3), (4), (8) | (3), (5), (6) | (3), (5), (7) | (3), (5), (8) | (3), (6), (7) |
| (3), (6), (8) | (4), (5), (6) | (4), (5), (7) | (4), (5), (8) | (4), (6), (7) | (4), (6), (8) |
| (5), (6), (7) | (5), (6), (8) | (5), (7), (8) | (6), (7), (8) | | |

| | | | |
|---|---|---|---|
| (1), (2), (3), (4) | (1), (2), (3), (5) | (1), (2), (3), (6) | (1), (2), (3), (7) |
| (1), (2), (3), (8) | (1), (2), (4), (5) | (1), (2), (4), (6) | (1), (2), (4), (7) |
| (1), (2), (4), (8) | (1), (2), (5), (6) | (1), (2), (5), (7) | (1), (2), (5), (8) |
| (1), (2), (6), (7) | (1), (2), (6), (8) | (1), (2), (7), (8) | (1), (3), (4), (5) |
| (1), (3), (4), (6) | (1), (3), (4), (7) | (1), (3), (4), (8) | (1), (3), (5), (6) |
| (1), (3), (5), (7) | (1), (3), (5), (8) | (1), (3), (6), (7) | (1), (3), (6), (8) |
| (1), (3), (7), (8) | (1), (4), (5), (6) | (1), (4), (5), (7) | (1), (4), (5), (8) |
| (1), (4), (6), (7) | (1), (4), (6), (8) | (1), (4), (7), (8) | (1), (5), (6), (7) |
| (1), (5), (6), (8) | (1), (5), (7), (8) | (1), (6), (7), (8) | (2), (3), (4), (5) |
| (2), (3), (4), (6) | (2), (3), (4), (7) | (2), (3), (4), (8) | (2), (3), (5), (6) |
| (2), (3), (5), (7) | (2), (3), (5), (8) | (2), (3), (6), (7) | (2), (3), (6), (8) |
| (2), (3), (7), (8) | (2), (4), (5), (6) | (2), (4), (5), (7) | (2), (4), (5), (8) |
| (2), (4), (6), (7) | (2), (4), (6), (8) | (2), (4), (7), (8) | (2), (5), (6), (7) |
| (2), (5), (6), (8) | (2), (5), (7), (8) | (2), (6), (7), (8) | (3), (4), (5), (6) |
| (3), (4), (5), (7) | (3), (4), (5), (8) | (3), (4), (6), (7) | (3), (4), (6), (8) |
| (3), (4), (7), (8) | (3), (5), (6), (7) | (3), (5), (6), (8) | (3), (5), (7), (8) |
| (3), (6), (7), (8) | (4), (5), (6), (7) | (4), (5), (6), (8) | (4), (5), (7), (8) |
| (4), (6), (7), (8) | (5), (6), (7), (8) | (1), (2), (3), (4), (5) | (1), (2), (3), (4), (6) |
| (1), (2), (3), (4), (7) | (1), (2), (3), (4), (8) | (1), (2), (3), (5), (6) | (1), (2), (3), (5), (7) |
| (1), (2), (3), (5), (8) | (1), (2), (3), (6), (7) | (1), (2), (3), (6), (8) | (1), (2), (3), (7), (8) |
| (1), (2), (4), (5), (6) | (1), (2), (4), (5), (7) | (1), (2), (4), (5), (8) | (1), (2), (4), (6), (7) |
| (1), (2), (4), (6), (8) | (1), (2), (4), (7), (8) | (1), (2), (5), (6), (7) | (1), (2), (5), (6), (8) |
| (1), (2), (5), (7), (8) | (1), (2), (6), (7), (8) | (1), (3), (4), (5), (6) | (1), (3), (4), (5), (7) |
| (1), (3), (4), (5), (8) | (1), (3), (4), (6), (7) | (1), (3), (4), (6), (8) | (1), (3), (4), (7), (8) |
| (1), (3), (5), (6), (7) | (1), (3), (5), (6), (8) | (1), (3), (5), (7), (8) | (1), (3), (6), (7), (8) |
| (1), (4), (5), (6), (7) | (1), (4), (5), (6), (8) | (1), (4), (5), (7), (8) | (1), (4), (6), (7), (8) |
| (1), (5), (6), (7), (8) | (2), (3), (4), (5), (6) | (2), (3), (4), (5), (7) | (2), (3), (4), (5), (8) |
| (2), (3), (4), (6), (7) | (2), (3), (4), (6), (8) | (2), (3), (4), (7), (8) | (2), (3), (5), (6), (7) |
| (2), (3), (5), (7), (8) | (2), (3), (6), (7), (8) | (2), (4), (5), (6), (7) | (2), (4), (5), (6), (8) |
| (2), (4), (5), (7), (8) | (2), (4), (6), (7), (8) | (2), (5), (6), (7), (8) | (3), (4), (5), (6), (7) |
| (3), (4), (5), (6), (8) | (3), (4), (5), (7), (8) | (3), (4), (6), (7), (8) | (3), (5), (6), (7), (8) |
| (4), (5), (6), (7), (8) | (1), (2), (3), (4), (5), (6) | (1), (2), (3), (4), (5), (7) | (1), (2), (3), (4), (5), (8) |
| (1), (2), (3), (4), (6), (7) | (1), (2), (3), (4), (6), (8) | (1), (2), (3), (4), (7), (8) | (1), (2), (3), (5), (6), (7) |
| (1), (2), (3), (5), (6), (8) | (1), (2), (3), (5), (7), (8) | (1), (2), (3), (6), (7), (8) | (1), (2), (4), (5), (6), (7) |
| (1), (2), (4), (5), (6), (8) | (1), (2), (4), (5), (7), (8) | (1), (2), (4), (6), (7), (8) | (1), (2), (5), (6), (7), (8) |
| (1), (3), (4), (5), (6), (7) | (1), (3), (4), (5), (6), (8) | (1), (3), (4), (5), (7), (8) | (1), (3), (4), (6), (7), (8) |
| (1), (3), (5), (6), (7), (8) | (1), (4), (5), (6), (7), (8) | (2), (3), (4), (5), (6), (7) | (2), (3), (4), (5), (6), (8) |
| (2), (3), (4), (5), (7), (8) | (2), (3), (4), (6), (7), (8) | (2), (3), (5), (6), (7), (8) | (2), (4), (5), (6), (7), (8) |
| (3), (4), (5), (6), (7), (8) | | | |

| | | |
|---|---|---|
| (1), (2), (3), (4), (5), (6), (7) | (1), (2), (3), (4), (5), (6), (8) | (1), (2), (3), (4), (5), (7), (8) |
| (1), (2), (3), (4), (6), (7), (8) | (1), (2), (3), (5), (6), (7), (8) | (1), (2), (4), (5), (6), (7), (8) |
| (1), (3), (4), (5), (6), (7), (8) | (2), (3), (4), (5), (6), (7), (8) | (1), (2), (3), (4), (5), (6), (7), (8) |

In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5; the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21; the MLP comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 22-24; and the at least two indigoidine transporter proteins comprise any combination as shown in Table 3. In some embodiments, the at least two indigoidine transporter proteins comprise SEQ ID NO:25 and SEQ ID NO:26. In some embodiments, the at least two indigoidine transporter proteins comprise SEQ ID NO:27 and SEQ ID NO:28. In some embodiments, the at least two indigoidine transporter proteins comprise SEQ ID NO:29, SEQ ID NO:31, and SEQ ID NO: 32. In some embodiments, the at least two indigoidine transporter proteins comprise SEQ ID NO: 26 and SEQ ID NO:27. In some embodiments, the at least two indigoidine transporter proteins comprise SEQ ID NO:26 and SEQ ID NO:28. In some embodiments, the at least two indigoidine transporter proteins comprise SEQ ID NO:26 and SEQ ID NO:29. In some embodiments, the at least two indigoidine transporter proteins comprise SEQ ID NO:26 and SEQ ID NO:30. In some embodiments, the at least two indigoidine transporter proteins comprise SEQ ID NO:31 and SEQ ID NO: 27. In some embodiments, the at least two indigoidine transporter proteins comprise SEQ ID NO: 26 and SEQ ID NO:32.

In some embodiments, the heterologous host cell is capable of expressing an INDS, a PPTase, and an MLP according to any of Combinations (1A)-(195) in Table 2, and is further capable of expressing at least two indigoidine transporter proteins according to a combination as shown in Table 3. In some embodiments, the heterologous host cell is capable of expressing an INDS, a PPTase, and an MLP according to any of Combinations (1A)-(195) in Table 2, and is further capable of expressing a first indigoidine transporter protein comprising at least 90% sequence identity to SEQ ID NO:25 and a second indigoidine transporter protein comprising at least 90% sequence identity to SEQ ID NO:26. In some embodiments, the heterologous host cell is capable of expressing an INDS, a PPTase, and an MLP according to any of Combinations (1A)-(195) in Table 2, and is further capable of expressing a first indigoidine transporter protein comprising at least 90% sequence identity to SEQ ID NO:27 and a second indigoidine transporter protein comprising at least 90% sequence identity to SEQ ID NO:28. In some embodiments, the heterologous host cell is capable of expressing an INDS, a PPTase, and an MLP according to any of Combinations (1A)-(195) in Table 2, and is further capable of expressing a first indigoidine transporter protein comprising at least 90% sequence identity to SEQ ID NO:29, a second indigoidine transporter protein comprising at least 90% sequence identity to SEQ ID NO:31, and a third indigoidine transporter protein comprising at least 90% sequence identity to SEQ ID NO: 32.

In some embodiments, the heterologous host cell is capable of expressing an INDS, a PPTase, and an MLP according to any of Combinations (1A)-(195) in Table 2, and is further capable of expressing a first indigoidine transporter protein comprising at least 90% sequence identity to SEQ ID NO: 26 and a second indigoidine transporter protein comprising at least 90% sequence identity to SEQ ID NO: 27. In some embodiments, the heterologous host cell is capable of expressing an INDS, a PPTase, and an MLP according to any of Combinations (1A)-(195) in Table 2, and is further capable of expressing a first indigoidine transporter protein comprising at least 90% sequence identity to SEQ ID NO: 26 and a second indigoidine transporter protein comprising at least 90% sequence identity to SEQ ID NO:28. In some embodiments, the heterologous host cell is capable of expressing an INDS, a PPTase, and an MLP according to any of Combinations (1A)-(195) in Table 2, and is further capable of expressing a first indigoidine transporter protein comprising at least 90% sequence identity to SEQ ID NO: 26 and a second indigoidine transporter protein comprising at least 90% sequence identity to SEQ ID NO:29. In some embodiments, the heterologous host cell is capable of expressing an INDS, a PPTase, and an MLP according to any of Combinations (1A)-(195) in Table 2, and is further capable of expressing a first indigoidine transporter protein comprising at least 90% sequence identity to SEQ ID NO: 26 and a second indigoidine transporter protein comprising at least 90% sequence identity to SEQ ID NO:30. In some embodiments, the heterologous host cell is capable of expressing an INDS, a PPTase, and an MLP according to any of Combinations (1A)-(195) in Table 2, and is further capable of expressing a first indigoidine transporter protein comprising at least 90% sequence identity to SEQ ID NO: 26 and a second indigoidine transporter protein comprising at least 90% sequence identity to SEQ ID NO:31. In some embodiments, the heterologous host cell is capable of expressing an INDS, a PPTase, and an MLP according to any of Combinations (1A)-(195) in Table 2, and is further capable of expressing a first indigoidine transporter protein comprising at least 90% sequence identity to SEQ ID NO: 26 and a second indigoidine transporter protein comprising at least 90% sequence identity to SEQ ID NO:32.

### Nucleic Acids

In some embodiments, the present disclosure further provides a nucleic acid encoding any of the proteins described herein, e.g., INDS, PPTase, and/or MLP provided herein. In some embodiments, the nucleic acid is operably linked to a heterologous regulatory element. The “heterologous regulatory element” is a regulatory element that is not naturally found to be associated or operably linked with the referenced nucleic acid. In some embodiments, the heterologous regulatory element comprises a promoter, an enhancer, a silencer, a response element, or a combination thereof. In some embodiments, the heterologous regulatory element is a bacterial regulatory element. Non-limiting examples of bacterial regulatory elements include the T7 promoter, Sp6 promoter, lac promoter, araBad promoter, trp promoter, and Ptac promoter. Further examples of regulatory elements can be found, e.g., using the PRODORIC2 database.

In some embodiments, the present disclosure provides a nucleic acid encoding an INDS comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any of SEQ ID NOs: 1-8, wherein the nucleic acid is operably linked to a heterologous regulatory element. In some embodiments, the present disclosure provides a nucleic acid encoding an INDS comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:1, wherein the nucleic acid is operably linked to a heterologous regulatory element. In some embodiments, the present disclosure provides a nucleic acid encoding an INDS comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:2, wherein the nucleic acid is operably linked to a heterologous regulatory element. In some embodiments, the present disclosure provides a nucleic acid encoding an INDS comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:3, wherein the nucleic acid is operably linked to a heterologous regulatory element. In some embodiments, the present disclosure provides a nucleic acid encoding an INDS comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO: 4, wherein the nucleic acid is operably linked to a heterologous regulatory element. In some embodiments, the present disclosure provides a nucleic acid encoding an INDS comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to SEQ ID NO:5, wherein the nucleic acid is operably linked to a heterologous regulatory element. In some embodiments, the heterologous regulatory element comprises a promoter.

In some embodiments, the present disclosure provides a vector comprising the nucleic acid encoding the INDS. In some embodiments, the vector further comprises a nucleic acid encoding a PPTase, an MLP, and/or an indigoidine transporter protein as described herein, e.g., a PPTase comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21, an MLP comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 22-24; and/or an indigoidine transporter protein comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 25-32. In some embodiments, the nucleic acid encoding the PPTase, the MLP, and/or the indigoidine transporter protein is operably linked to a heterologous regulatory element. In some embodiments, the heterologous regulatory element comprises a promoter. In some embodiments, the vector is an expression vector. In some embodiments, the vector is a bacterial expression vector. In some embodiments, the vector is a fungal expression vector. In some embodiments, the vector is an E. coli, Corynebacterium, Bacillus, Ralstonia, Zymomonas, Staphylococcus, Pichia (e.g., Pichia pastoris), Saccharomyces, Candida (e.g., Candida albicans), Yarrowia, or Aspergillus expression vector.

In some embodiments, the present disclosure provides one or more vectors comprising: (i) a nucleic acid encoding the INDS as described herein, (ii) a nucleic acid encoding the PPTase as described herein, (iii) a nucleic acid encoding the MLP as described herein; and (iv) a nucleic acid encoding the indigoidine transporter protein as described herein. In some embodiments, (i) is on a first vector, (ii) is on a second vector, (iii) is on a third vector; and (iv) is on a fourth vector. In some embodiments, (i) and (ii) are on a first vector, (iii) is on a second vector, and (iv) is a third vector. In some embodiments, (i) and (iii) are on a first vector, (ii) is on a second vector; and (iv) is a third vector. In some embodiments, (i) and (iv) are on a first vector, (ii) is on a second vector, and (iii) is a third vector. In some embodiments, (ii) and (iii) are on a first vector, (i) is on a second vector, and (iv) is a third vector. In some embodiments, (ii) and (iv) are on a first vector, (i) is on a second vector, and (iii) is a third vector. In some embodiments, (iii) and (iv) are on a first vector, (i) is on a second vector, and (iii) is a third vector. In some embodiments, (i), (ii), and (iii) are on a first vector, and (iv) is on a second vector. In some embodiments, (i), (ii), and (iv) are on a first vector, and (iii) is on a second vector. In some embodiments, (i), (iii), and (iv) are on a first vector, and (ii) is on a second vector. In some embodiments, (ii), (iii), and (iv) are on a first vector, and (i) is on a second vector. In some embodiments, (i) (ii), (iii), and (iv) are on the same vector. In embodiments where more than one nucleic acid is on the same vector, each nucleic acid may be operably linked to a separate regulatory element (e.g., promoter), thereby allowing the expression of each nucleic acid to be controlled separately. Alternatively, a single regulatory element may be operably linked to multiple nucleic acids, thereby allowing simultaneous control of the multiple nucleic acids.

In some embodiments, the one or more vectors comprise a nucleic acid encoding the INDS as described herein and a nucleic acid encoding the PPTase as described herein, wherein the INDS and the PPTase comprise any of Combinations (1)-(65) in Table 1. In some embodiments, the one or more vectors comprise a nucleic acid encoding the INDS as described herein, a nucleic acid encoding the PPTase, and a nucleic acid encoding the MLP as described herein, wherein the INDS, the PPTase, and the MLP comprise any of Combinations (1)-(195) in Table 2. In some embodiments, the one or more vectors comprise a nucleic acid encoding the INDS as described herein, a nucleic acid encoding the PPTase, a nucleic acid encoding the MLP as described herein, and a nucleic acid encoding one or more indigoidine transporter proteins as described herein, wherein the INDS, the PPTase, and the MLP comprise any of Combinations (1)-(195) in Table 2, and wherein the indigoidine transporter protein comprises at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 25-32, or the indigoidine transporter proteins comprises a combination as shown in Table 3. In some embodiments, the INDS, PPTase, and MLP (if present), each independently comprises at least 75%, at least 80%, at least 85%, at least 90%, at least 91%, at least 92%, at least 93%, at least 94%, at least 95%, at least 96%, at least 97%, at least 98%, at least 99%, or 100% sequence identity to the sequences indicated in each combination of Table 1 or Table 2.

Non-limiting examples of vectors are provided herein. In some embodiments, the vector is a bacterial expression vector, e.g., pET, pGEX, pQE, pBAD, pMAL, pBluescript, pNH, pZ, pTrc99a, pEC-XT99A, pVWEx1, and the like. In some embodiments, the vector is a fungal expression vector, e.g., pGAPZ, pPICHOLI, pPIC, pYES, and the like. In some embodiments, the vector is suitable for expression of the INDS and the PPTase and/or MLP in a host cell.

Methods of introducing nucleic acids and/or vectors into host cells are known to one of ordinary skill in the art. Non-limiting exemplary methods for introducing nucleic acids and/or vectors into host cells include electroporation, conjugation, transduction, natural transformation, calcium phosphate precipitation, DEAE-dextran mediated transfection, liposome-mediated transfection, and particle bombardment. The nucleic acid or vector typically further includes a selectable marker, e.g., neomycin resistance, ampicillin resistance, tetracycline resistance, chloramphenicol resistance, kanamycin resistance, hygromycin resistance, G418 resistance, bleomycin resistance, zeocin resistance, and the like.

In some embodiments, the nucleic acids described herein, e.g., encoding an INDS, PPTase, MLP, and/or indigoidine transporter protein described herein, are stably integrated into the genome of the host cell. In some embodiments, the nucleic acids are integrated into the host cell genome using a CRISPR system. In some embodiments, the nucleic acids are integrated into the host cell genome using homologous recombination, e.g., by the bacteriophage lambda Red system, transposon systems, and methods utilizing enzymes such as flippase, endonuclease, and/or recombinase.

### Host Modifications

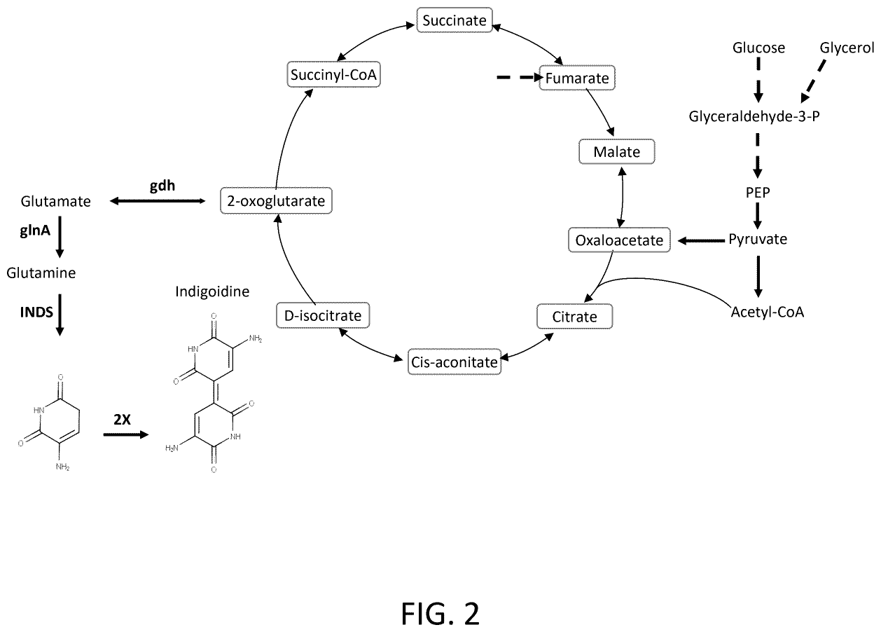

In some embodiments, the heterologous host cell, which is capable of expressing the INDS and the PPTase and/or MLP as described herein, further comprises a modification to increase indigoidine production by the INDS in the host cell. In some embodiments, the modification comprises altering the levels of one or more endogenous proteins of the host cell. In some embodiments, the modification increases efflux of the indigoidine production pathway in the host cell. An exemplary indigoidine production pathway from glucose or glycerol as the carbon source is illustrated in FIG. 2. In some embodiments, the modification increases the level of intermediates and/or precursors that feed into the indigoidine production pathway. In some embodiments, the modification increases the production of glutamine and/or glutamate by the host cell. In some embodiments, the modification increases the available pool of redox and/or cofactors required for indigoidine biosynthesis. In some embodiments, the modification increases uptake of the carbon source for indigoidine biosynthesis, e.g., glycerol or glucose, into the host cell. In some embodiments, the modification reduces or eliminates pathways that compete with indigoidine biosynthesis. In some embodiments, the modification reduces or eliminates byproducts that hinder indigoidine biosynthesis, e.g., toxic byproducts. In some embodiments, the modification increases transport of the produced indigoidine into the cell culture medium.

In some embodiments, the modification increases the levels of one or more proteins that increase indigoidine production, e.g., by expressing or upregulating an endogenous gene encoding the one or more proteins in the host cell, and/or by introducing or overexpressing an exogenous gene encoding the one or more proteins into the host cell. In some embodiments, the modification decreases the levels of one or more proteins that decrease indigoidine production, e.g., by downregulating, disrupting, or deleting an endogenous gene encoding the one or more proteins of the host cell, and/or by degrading or promoting degradation of the one or more proteins.

In some embodiments, the modification comprises increasing the levels of a protein that:
a) catalyzes the conversion of acetate into acetyl-CoA, e.g., acetyl-coenzyme A synthetase;
b) catalyzes the reversible reductive amination of alpha-ketoglutarate and ammonia to glutamate, e.g., NAD(P) H-specific glutamate dehydrogenase;
c) catalyzes the ATP-dependent biosynthesis of glutamine from glutamate and ammonia, e.g., glutamine synthetase;
d) catalyzes the conversion of isocitrate to 2-oxoglutarate, e.g., isocitrate dehydrogenase;
e) catalyzes the conversion of pyruvate into acetyl-CoA, e.g., dihydrolipoyl dehydrogenase;
f) catalyzes the irreversible carboxylation of phosphoenolpyruvate (PEP) to form oxaloacetate, e.g., phosphoenolpyruvate carboxylase;
g) catalyzes the formation of oxaloacetate from pyruvate, e.g., pyruvate carboxylase;
h) controls the transcription of genes in the glyoxylate pathway genes, for example the aceBAK operon of E. coli, e.g., transcriptional repressor IclR;
i) catalyzes the transhydrogenation between NADH and NADP, e.g., NAD(P) transhydrogenase subunit alpha or NAD(P) transhydrogenase subunit beta;
j) catalyzes the phosphorylation of pantothenate, e.g., Type III pantothenate kinase;
k) allows glucose uptake into the cell, e.g., glucose facilitated diffusion protein;
l) catalyzes the formation of D-glucose 6-phosphate from D-glucose, e.g., glucokinase; and/or
m) has transposase activity.

In some embodiments, the modification comprises increasing the levels of a protein selected from acetyl-coenzyme A synthetase, NAD(P) H-specific glutamate dehydrogenase, glutamine synthetase, isocitrate dehydrogenase, dihydrolipoyl dehydrogenase, phosphoenolpyruvate carboxylase, pyruvate carboxylase, transcriptional repressor IclR, NAD(P) transhydrogenase subunit alpha, NAD(P) transhydrogenase subunit beta, Type III pantothenate kinase, glucose facilitated diffusion protein, glucokinase, S-formylglutathione hydrolase, transposase (e.g., ydcC and yncI), and any combination thereof. Methods of increasing protein levels in a host cell are known to one of ordinary skill in the art. In some embodiments, the modification comprises increasing ydcC expression in the host cell. In some embodiments, the modification comprises increasing yncI expression in the host cell. In some embodiments, the levels of the protein are increased by upregulation of the endogenous gene in the host cell. In some embodiments, the levels of the protein are increased by introducing an exogenous nucleic acid encoding the protein into the host cell for overexpression of the protein. Introduction of exogenous nucleic acids is further described herein.

In some embodiments, the modification comprises decreasing the levels of a protein that:
a) catalyzes the conversion of pyruvate into D-lactate, e.g., D-lactate dehydrogenase
b) catalyzes the oxidative decarboxylation of pyruvate to form acetate and carbon dioxide, e.g., ubiquinone-dependent pyruvate dehydrogenase;
c) catalyzes the sequential NADH-dependent reduction of acetyl-CoA to acetaldehyde and then to ethanol, e.g., aldehyde-alcohol dehydrogenase;
d) catalyzes the reversible interconversion of acetyl-CoA and acetyl phosphate, e.g., phosphate acetyltransferase;
e) catalyzes the reversible interconversion of acetate and acetyl phosphate, e.g., acetate kinase;
f) regulates or is involved in regulation of glutamine synthetase GlnA, e.g., bifunctional glutamine synthetase adenylyltransferase;
g) catalyzes the conversion of D-glutamine into D-glutamate, e.g., glutaminase 1 or glutaminase 2;
h) catalyzes the conversion of L-glutamine and 2-oxoglutarate into molecules of L-glutamate, e.g., glutamate synthase [NADPH] large chain or glutamate synthase [NADPH] small chain;
i) represses aerobic enzymes under anaerobic conditions, e.g., aerobic respiration control protein (arcA);
j) catalyzes the conversion of 2-oxoglutarate to succinyl-CoA, e.g., 2-oxoglutarate dehydrogenase E1 component or dihydrolipoyllysine-residue succinyltransferase component of 2-oxoglutarate dehydrogenase complex;
k) catalyzes the reversible oxidation of malate to oxaloacetate, e.g., malate dehydrogenase;
l) catalyzes the irreversible formation of pyruvate from PEP, e.g., pyruvate kinase I;
m) catalyzes the conversion of adenine into hypoxanthine, e.g., adenine deaminase;
n) catalyzes the conversion of cytidine into uridine, e.g., cytidine deaminase;
o) catalyzes the conversion of guanine into xanthine, e.g., guanine deaminase;
p) forms pores that allow passive diffusion of small molecules across the outer membrane, e.g., outer membrane porin C;
q) catalyzes the formation of a quinone and nitrite from a quinol and nitrate, e.g., respiratory nitrate reductase 1 alpha chain;
r) catalyzes the formation of L-aspartate from L-asparagine, e.g., L-asparaginase 2;
s) catalyzes the deamination of enamine/imine intermediates to yield 2-ketobutyrate and ammonia, e.g., 2-iminobutanoate/2-iminopropanoate deaminase;
t) catalyzes the hydrolytic deamination of adenosine and 2-deoxyadenosine, e.g., adenosine deaminase;
u) catalyzes the hydrolytic deamination of cytosine to uracil, e.g., cytosine deaminase;
v) forms part of an ABC transporter involved in dipeptide transport, for example DppABCDF, e.g., dipeptide-binding protein; and/or
w) forms a component of the oligopeptide permease, e.g., periplasmic oligopeptide-binding protein.

In some embodiments, the modification comprises decreasing the levels of a protein selected from D-lactate dehydrogenase, ubiquinone-dependent pyruvate dehydrogenase (e.g., poxB), aldehyde-alcohol dehydrogenase, phosphate acetyltransferase, acetate kinase, bifunctional glutamine synthetase adenylyltransferase, glutaminase 1, glutaminase 2, glutamate synthase [NADPH] large chain, glutamate synthase [NADPH] small chain, aerobic respiration control protein (arcA), 2-oxoglutarate dehydrogenase E1 component, dihydrolipoyllysine-residue succinyltransferase component of 2-oxoglutarate dehydrogenase complex, malate dehydrogenase, pyruvate kinase I, adenine deaminase, cytidine deaminase, guanine deaminase, outer membrane porin C, respiratory nitrate reductase 1 alpha chain, L-asparaginase 2, 2-iminobutanoate/2-iminopropanoate deaminase, adenosine deaminase, cytosine deaminase, dipeptide-binding protein, periplasmic oligopeptide-binding protein, fused glutamine synthetase deadenylase/glutamine synthetase adenylyltransferase, L-glutamine ABC transporter periplasmic binding protein, L-glutamine ABC transporter membrane subunit, L-glutamine ABC transporter ATP binding subunit, pseudouridine-5′-phosphate glycosidase, pseudouridine kinase, pseudouridine transporter, tautomerase (e.g., pptA), and any combination thereof. In some embodiments, the modification comprises deletion of arcA in the host cell. In some embodiments, the modification comprises deletion of pptA in the host cell. In some embodiments, the modification comprises deletion of poxB in the host cell. Methods of decreasing protein levels in a host cell are known to one of ordinary skill in the art. In some embodiments, the levels of the protein are decreased by reducing transcription and/or translation of the endogenous gene in the host cell. In some embodiments, the levels of the protein are decreased by a loss-of-function mutation or deletion of the endogenous gene in the host cell. In some embodiments, the levels of the protein are decreased by RNA interference.

In some embodiments, the modification comprises altering the expression of DNA gyrase subunit A. In some embodiments, the expression of DNA gyrase subunit A is altered by a mutation in its regulatory sequence that increased glutamate production. In some embodiments, the mutation increases levels of DNA gyrase subunit A. In some embodiments, the mutation decreases levels of DNA gyrase subunit A. In some embodiments, the modification comprises altering the expression of cytochrome d oxidase cydAB. In some embodiments, the expression of cydAB is altered by a mutation in cydAB. In some embodiments, the mutation increases levels of cydAB. In some embodiments, the mutation decreases levels of cydAB.

In some embodiments, the heterologous host cell is an E. coli cell. Exemplary E. coli proteins that can be modified as described herein are provided in Table 3. One of ordinary skill in the art would be capable of determining the analogous genes in any of the host cell organisms provided herein.

TABLE 3
Exemplary E. coli Proteins

| | Gene | |
|---|---|---|
| Protein/Gene Name | Abbreviation | Accession ID |
| D-lactate dehydrogenase | ldhA | MCV5771625.1 |
| ubiquinone-dependent pyruvate dehydrogenase | poxB | CAD6018048.1 |
| Aldehyde-alcohol dehydrogenase | adhE | 6AHC_A |
| Phosphate acetyltransferase | pta | NC_000913.3 |
| Acetate kinase | ackA | NP_416799.1 |
| Acetyl-coenzyme A synthetase | acs | NP_418493.1 |
| NAD(P)H-specific glutamate dehydrogenase | gdh | NP_416275.1 |
| Glutamine synthetase | glnA | NP_418306.1 |
| Bifunctional glutamine synthetase adenylyltransferase | glnE | NP_417525.1 |
| Glutaminase 1 | glsA1 | NP_415018.1 |
| Glutaminase 2 | glsA2 | NP_416041.1 |
| Glutamate synthase [NADPH] large chain | gltB | NP_417679.2 |
| Glutamate synthase [NADPH] small chain | gltD | NP_417680.1 |
| Isocitrate dehydrogenase | icd | NP_415654.1 |
| Aerobic respiration control protein | arcA | NP_418818.1 |
| 2-oxoglutarate dehydrogenase E1 component | sucA | NP_415254.1 |
| Dihydrolipoyllysine-residue succinyltransferase component | sucB | NP_415255.1 |
| of 2-oxoglutarate dehydrogenase complex | | |
| Dihydrolipoyl dehydrogenase | lpdA | NP_414658.1 |
| Malate dehydrogenase | mdh | NP_417703.1 |
| Phosphoenolpyruvate carboxylase | PEPC/ppc | NP_418391.1 |
| pyruvate carboxylase | pyc | AAF09095.1 |
| Transcriptional repressor IclR | iclR | NP_418442.2 |
| Pyruvate kinase I | pykF | NP_416191.1 |
| NAD(P) transhydrogenase subunit alpha | pntA | NP_416120.1 |
| NAD(P) transhydrogenase subunit beta | pntB | NP_416119.1 |
| Type III pantothenate kinase | panK | NP_387951.2 |
| Glucose facilitated diffusion protein | glf | WP_011240287.1 |
| Glucokinase | glK | AAA27694.1 |
| Adenine deaminase | adeD | QED71488.1 |
| Cytidine deaminase | cdd | NP_416648.1 |
| Guanine deaminase | guaD | NP_417359.1 |
| Outer membrane porin C | ompC | NP_416719.1 |
| Respiratory nitrate reductase 1 alpha chain | narG | NP_415742.1 |
| L-asparaginase 2 | ansB | NP_417432.1 |
| 2-iminobutanoate/2-iminopropanoate deaminase | ridA | NP_418664.2 |
| Adenosine deaminase | add | NP_416140.1 |
| Cytosine deaminase | codA | NP_414871.1 |
| Dipeptide-binding protein | dppA | NP_418001.1 |
| Periplasmic oligopeptide-binding protein | oppA | NP_415759.1 |
| Fused glutamine synthetase deadenylase/glutamine | glnE | |
| synthetase adenylyltransferase | | |
| L-glutamine ABC transporter periplasmic binding protein | glnH | |
| L-glutamine ABC transporter membrane subunit | glnP | |
| L-glutamine ABC transporter ATP binding subunit | glnQ | |
| pseudouridine-5′-phosphate glycosidase | psuG | |
| putative pseudouridine kinase | psuK | |
| putative pseudouridine transporter | psuT | |
| tautomerase PptA | pptA | |
| S-formylglutathione hydrolase | yeiG | |
| DNA gyrase subunit A | gyrA | |

A variety of microorganisms may be suitable as the host cell described herein. In some embodiments, the host cell is a prokaryotic cell. In some embodiments, the host cell is a eukaryotic cell. In some embodiments, the host cell is bacteria. In some embodiments, the bacteria is Escherichia, Corynebacterium, Pseudomonas, Bacillus, Ralstonia, Zymomonas, Staphylococcus, Clostridium, Salmonella, Rhodococcus, Enterococcus, Alcaligenes, Klebsiella, Paenibacillus, Arthrobacter, Brevibacterium, Lactobacillus, or Lactococcus. In some embodiments, the bacteria is Escherichia, Corynebacterium, Bacillus, Ralstonia, Zymomonas, or Staphylococcus. In some embodiments, the bacteria is Escherichia coli, Corynebacterium glutamicum, Pseudomonas aeruginosa, Bacillus subtilis, Ralstonia eutropha, Zymomonas mobilis, or Staphylococcus aureus. In some embodiments, the host cell is fungi. In some embodiments, the fungi is Saccharomyces, Pichia, Yarrowia, Aspergillus, or Candida. In some embodiments, the fungi is Saccharomyces cerevisiae, Pichia pastoris, Yarrowia lipolytica, Aspergillus fumigatus, or Candida albicans. In some embodiments, the host cell is Pichia pastoris. In some embodiments, the host cell is E. coli. In some embodiments, the E. coli is an E. coli K-12 strain or derivative thereof. In some embodiments, the E. coli is an E. coli B strain or derivative thereof. In some embodiments, the E. coli is E. coli K-12 W3110, E. coli K-12 DH10b, E. coli K-12 DH1, E. coli K-12 MG1655, E. coli BW2952, E. coli B REL606, E. coli BL21, or E. coli BL21 (DE3). In some embodiments, the host cell is E. coli K-12 MG1655.

Further exemplary host cells include, but are not limited to, Escherichia coli, Saccharomyces cerevisiae, Saccharomyces kluyveri, Candida boidinii, Clostridium kluyveri, Clostridium acetobutylicum, Clostridium beijerinckii, Clostridium saccharoperbutylacetonicum, Clostridium perfringens, Clostridium difficile, Clostridium botulinum, Clostridium tyrobutyricum, Clostridium tetanomorphum, Clostridium tetani, Clostridium propionicum, Clostridium aminobutyricum, Clostridium subterminale, Clostridium sticklandii, Ralstonia eutropha, Mycobacterium bovis, Mycobacterium tuberculosis, Porphyromonas gingivalis, Arabidopsis thaliana, Thermus thermophilus, Pseudomonas aeruginosa, Pseudomonas putida, Pseudomonas stutzeri, Pseudomonas fluorescens, Rhodobacter spaeroides, Thermoanaerobacter brockii, Metallosphaera sedula, Leuconostoc mesenteroides, Chloroflexus aurantiacus, Roseiflexus castenholzii, Simmondsia chinensis, Acinetobacter calcoaceticus, Acinetobacter baylyi, Porphyromonas gingivalis, Sulfolobus tokodaii, Sulfolobus solfataricus, Sulfolobus acidocaldarius, Bacillus subtilis, Bacillus cereus, Bacillus megaterium, Bacillus brevis, Bacillus pumilus, Klebsiella pneumonia, Klebsiella oxytoca, Euglena gracilis, Treponema denticola, Moorella thermoacetica, Thermotoga maritima, Halobacterium salinarum, Geobacillus stearothermophilus, Aeropyrum pernix, Caenorhabditis elegans, Corynebacterium glutamicum, Acidaminococcus fermentans, Lactococcus lactis, Lactobacillus plantarum, Streptococcus thermophilus, Enterobacter aerogenes, Candida albicans, Aspergillus terreus, Pedicoccus pentosaceus, Zymomonas mobilus, Acetobacter pasteurians, Kluyveromyces lactis, Eubacterium barkeri, Bacteroides capillosus, Anaerotruncus colihominis, Natranaerobius thermophilusm, Campylobacter jejuni, Haemophilus influenzae, Serratia marcescens, Citrobacter amalonaticus, Myxococcus xanthus, Fusobacterium nuleatum, Penicillium chrysogenum, Nocardia iowensis, Nocardia farcinica, Streptomyces griseus, Schizosaccharomyces pombe, Geobacillus thermoglucosidasius, Salmonella typhimurium, Vibrio cholera, Heliobacter pylori, Nicotiana tabacum, Haloferax mediterranei, Agrobacterium tumefaciens, Achromobacter denitrificans, Fusobacterium nucleatum, Streptomyces clavuligenus, Acinetobacter baumanii, Lachancea kluyveri, Trichomonas vaginalis, Trypanosoma brucei, Pseudomonas stutzeri, Bradyrhizobium japonicum, Mesorhizobium loti, Nicotiana glutinosa, Vibrio vulnificus, Selenomonas ruminantium, Vibrio parahaemolyticus, Archaeoglobus fulgidus, Haloarcula marismortui, Pyrobaculum aerophilum, Mycobacterium smegmatis, Mycobacterium avium, Mycobacterium marinum, and Tsukamurella paurometabola.

In some embodiments, the host cell is E. coli, and the E. coli expresses an INDS and a PPTase according to any of the combinations in Table 1. In some embodiments, the host cell is E. coli, and the E. coli expresses an INDS, a PPTase, and a NRPS accessory protein, e.g., MLP, according to any of the combinations in Table 2. In embodiments, the E. coli is E. coli K-12 MG1655.

In embodiments, the host cell comprises E. coli K-12 MG1655 and expresses: an INDS comprising at least 90% sequence identity to SEQ ID NO:1; and a PPTase comprising at least 90% sequence identity to SEQ ID NO:9 and optionally comprises a D198G mutation, wherein the host cell (i) expresses an NRPS accessory protein comprising at least 90% sequence identity to any one of SEQ ID NOs: 22-24; (ii) expresses an indigoidine transporter protein comprising at least 90% sequence identity to any one of SEQ ID NO:25-32 or expresses a combination of indigoidine transporter proteins according to Table 3; (iii) comprises a mutation in the endogenous cydAB; (iv) comprises a deletion of the endogenous arcA; (v) comprises a deletion of the endogenous pptA; (vi) comprises a deletion of the endogenous ydcC; (vii) comprises a deletion of the endogenous yncI; (ix) comprises a deletion of the endogenous poxB; or (x) any combination of (i)-(ix).

In some embodiments, the host cell comprises E. coli K-12 MG1655 and (i) expresses the following: an INDS comprising at least 90% sequence identity to SEQ ID NO:1; a PPTase comprising at least 90% sequence identity to SEQ ID NO:9 and optionally comprises a D198G mutation; an NRPS accessory protein comprising at least 90% sequence identity to any one of SEQ ID NOs: 22-24; an indigoidine transporter protein comprising at least 90% sequence identity to any one of SEQ ID NO: 25-32 or a combination of indigoidine transporter proteins according to Table 3; (ii) comprises a deletion of one or more of the following endogenous genes: arcA, pptA, ydcC, yncI, and poxB; and (iii) comprises a mutation in the endogenous cydAB. In some embodiments, the indigoidine transporter protein comprises at least 90% sequence identity to SEQ ID NO:26. In some embodiments, the indigoidine transporter protein comprises a first protein comprising at least 90% sequence identity to SEQ ID NO:26 and a second protein comprising at least 90% sequence identity to any one of SEQ ID NOs: 25 and 27-32. In some embodiments, the genes expressed by the host cells are on a plasmid. In some embodiments, the genes expressed by the host cells are chromosomally integrated. Methods of chromosomal integration of exogenous nucleic acids are described herein and known to one of ordinary skill in the art.

### Fermentation Methods

In some embodiments, the present disclosure provides a method of making indigoidine, comprising expressing the INDS described herein in a heterologous host cell described herein. In some embodiments, the method further comprises expressing the PPTase and/or the MLP as described herein in the heterologous host cell. In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5. In some embodiments, the PPTase comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21. In some embodiments, the MLP comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 22-24. In some embodiments, the indigoidine transporter protein comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 25-32. In some embodiments, a combination of INDS and PPTase as shown in Table 1 is expressed in the heterologous host cell. In some embodiments, a combination of INDS, PPTase, and MLP as shown in Table 2 is expressed in the heterologous host cell. In some embodiments, the heterologous host cell expresses a combination of INDS and PPTase as shown in Table 1, and an indigoidine transporter protein of any one of SEQ ID NOs: 25-32 or a combination of indigoidine transporter proteins as shown in Table 3. In some embodiments, the heterologous host cell expresses a combination of INDS, PPTase, and MLP as shown in Table 2, and an indigoidine transporter protein of any one of SEQ ID NOs: 25-32 or a combination of indigoidine transporter proteins as shown in Table 3.

In some embodiments, the method comprises culturing the heterologous host cell in a culture medium prior to and/or during expression of the INDS and the PPTase, MLP, and/or indigoidine transporter protein as described herein. The appropriate culture medium for the host cell may be selected by one of ordinary skill in the art. For example, descriptions of various culture media may be found in “Manual of Methods for General Bacteriology” of the American Society for Bacteriology (Washington D.C., U.S.A., 1981). In some embodiments, the culture medium comprises a carbon source, a supplement, or a combination thereof. In some embodiments, the culture medium further comprises a cofactor and/or coreactant for the proteins described herein, e.g., ATP, MgCl₂, FMN, FAD, NADH, NADPH, or any combination thereof. In some embodiments, the presence of the carbon source and/or the supplement in the culture medium increases indigoidine production by the host cell. In some embodiments, the carbon source and/or the supplement feeds into the indigoidine production pathway, e.g., as illustrated in FIG. 2.

Exemplary carbon sources include, but are not limited to, glucose, galactose, fructose, mannose, isomaltose, xylose, maltose, arabinose, succinate, lignocellulose, molasses, cellobiose and 3-, 4-, or 5-oligomers thereof, methanol, ethanol, glycerol, formate, and fatty acids. In some embodiments, the carbon source comprises glucose, glycerol, sucrose, galactose, acetate, succinate, lignocellulose, molasses, or combinations thereof. In some embodiments, the carbon source is glucose. In some embodiments, the carbon source is glycerol. In some embodiments, the supplement comprises alpha-ketoglutarate, glutamate, glutamine, pantothenate, casamino acid, thiamine, biotin, riboflavin, trace metals, vitamins, or combinations thereof. In some embodiments, the supplement comprises a glutamate, glutamine, or a combination thereof. It was discovered that addition of glutamate in the culture medium provided higher yield than the precursor glutamine. A further discovery was that, in some embodiments, a combination of glutamine and glutamate provided higher yield than glutamine alone or glutamate alone.

In some embodiments, the host cell is cultured under aerobic, microaerobic, anaerobic or substantially anaerobic conditions. Exemplary aerobic, microaerobic, and anaerobic conditions are known to one of ordinary skill in the art. Briefly, anaerobic conditions refer to an environment devoid of oxygen. Substantially anaerobic conditions include, for example, a culture, batch fermentation or continuous fermentation such that the dissolved oxygen concentration in the medium remains between 0 and 10%. Substantially anaerobic conditions also include growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases.

In some embodiments, the host cell is cultured under conditions for large scale production of indigoidine. Exemplary growth procedures include, but are not limited to, fed-batch fermentation and batch separation; fed-batch fermentation and continuous separation; or continuous fermentation and continuous separation In some embodiments, the host cell is cultured in a continuous culture, i.e., providing the host cell with sufficient nutrients and culture medium to sustain and/or nearly sustain growth in an exponential phase. Continuous culture can be, e.g., for about 1, 2, 3, 4, 5, 6, 7 or more than 7 days, or about 1, 2, 3, 4, or more than 4 weeks, or up to several months. In some embodiments, the host cell is cultured for about 1 hour to about 24 hours, or about 2 hours to about 22 hours, or about 3 hours to about 20 hours, or about 4 hours to about 18 hours, or about 5 hours to about 16 hours, or about 5 hours to about 14 hours, or about 6 hours to about 12 hours, or about 8 hours to about 10 hours. In some embodiments, the host cell is cultured for a sufficient period of time to produce a desired amount of indigoidine.

Growth conditions for the host cells described herein are known to ordinary skill in the art. In some embodiments, the host cells are grown at a temperature of about 25° C. to about 40° C., or about 22° C. to about 38° C., or about 18° C. to about 45° C., or about 30° C. to about 55° C. In some embodiments, the host cell is a thermophilic organism, and the growth temperature is up to about 70° C. In some embodiments, the pH of the culture medium is about 4 to about 10, or about 5 to about 9.5, or about 5.5 to about 9, or about 6 to about 8.5, or about 6.5 to about 8, or about 7 to about 7.5.

In some embodiments, the method further comprises isolating the indigoidine, e.g., upon completion of the culture period. In some embodiments, the indigoidine is isolated from the culture medium, the host cell, a cell extract thereof, a whole culture thereof, or a combination thereof. As used herein, “whole culture” refers to the host cells and the culture medium in which they are cultured. As used herein, “cell extract” refers to a lysate of the cultured cells, which may include the culture medium and may be crude (unpurified), purified, or partially purified. Methods of lysing cells and purifying lysate are known to one of ordinary skill in the art, and exemplary methods are provided herein.

In some embodiments, the method comprises isolating the indigoidine from a cell extract of the host cell. In some embodiments, the host cells are lysed, e.g., mechanically, chemically, or enzymatically, and the soluble and insoluble components of the lysed cells are separated (e.g., via filtration and/or centrifugation) into lysate and pellet, respectively. In some embodiments, the method further comprises purifying the indigoidine from the cell extract. Exemplary purification methods include, but are not limited to, pervaporation, evaporation, filtration, membrane filtration (including diafiltration, nanofiltration, ultrafiltration, and microfiltration), membrane separation, reverse osmosis, electrodialysis, distillation, extractive distillation, reactive distillation, azeotropic distillation, crystallization and recrystallization, centrifugation, ion exchange chromatography, size exclusion chromatography, adsorption chromatography, carbon adsorption, or a combination thereof.

In some embodiments, the amount of indigoidine produced by the method described herein is quantified by an analytical quantification method. Exemplary analytical quantification methods include, not are not limited to, high performance liquid chromatography (HPLC), gas chromatography (GC), quantitative mass spectrometry, spectrophotometry, or combinations thereof. In some embodiments, the method described herein yields greater than 50%, greater than 60%, greater than 70%, greater than 80%, or greater than 90% indigoidine relative to the amount of byproducts produced in the method. In some embodiments, the method described herein yields about 50% to about 100%, or about 60% to about 99%, or about 70% to about 97%, or about 75% to about 95%, or about 80% to about 92%, or about 85% to about 90% indigoidine relative to the amount of byproducts produced in the method.

### Cell-Free Methods

In some embodiments, the present disclosure provides a cell-free method of making indigoidine, comprising contacting an INDS described herein with glutamine under conditions sufficient to produce indigoidine. In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5. In some embodiments, the INDS is produced by a heterologous host cell described herein. In some embodiments, the INDS is produced from a heterologous host cell that further expresses a PPTase as described herein, thereby producing an activated INDS. In some embodiments, the INDS is an activated INDS as described herein, i.e., comprising a PPT group in its carrier domain.

In some embodiments, the INDS produced by the host cell is inactive INDS. In some embodiments, the host cell expresses inactive INDS at higher levels than activated INDS. In some embodiments, the cell-free method further comprises a step of activating the INDS in vitro, prior to contacting the INDS with glutamine. In some embodiments, the activating comprises contacting the INDS with a PPTase and coenzyme A, thereby forming the activated INDS comprising the PPT group. In some embodiments, the PPTase is a PPTase described herein, comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21. In some embodiments, the INDS and PPTase of the cell-free method are any of the combinations as shown in Table 1.

In some embodiments, the contacting of the INDS with glutamine in the presence of one or more components required to form indigoidine, i.e., ATP, MgCl₂, FMN, or combination thereof. In some embodiments, the contacting of the INDS with glutamine is further in the presence of an MLP. In some embodiments, the MLP is an MLP described herein, comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 22-24. In some embodiments, the INDS, PPTase, and MLP of the cell-free method are any of the combinations as shown in Table 2. In some embodiments, the contacting of the INDS with glutamine is further in the presence of ADP, a polyphosphate kinase (PPK), inorganic polyphosphate, or a combination thereof. In some embodiments, the ADP, PPK, and inorganic phosphate generate ATP, which is used by the INDS to produce indigoidine. In some embodiments, the PPK is provided as purified PPK enzyme. In some embodiments, the PPK is provided in a cell lysate.

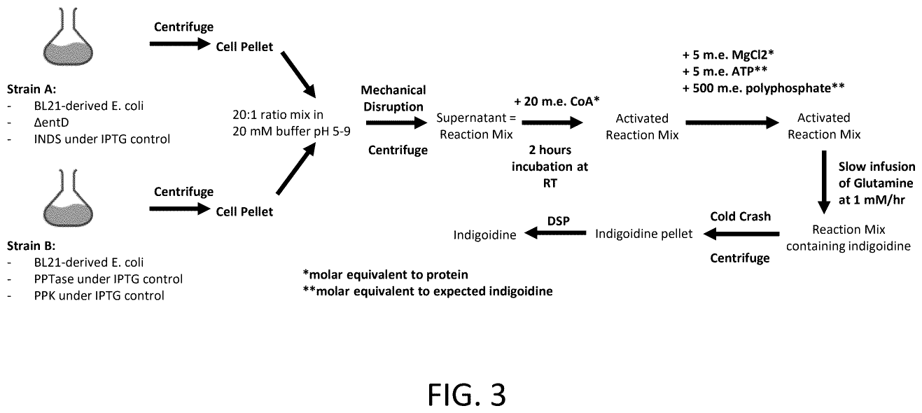

A non-limiting, exemplary cell-free method is illustrated in FIG. 3. An exemplary INDS, e.g., any of the INDSs described herein, is produced in “apo” form in a first E. coli strain (Strain A) containing a knockout of the entD PPTase. Without being bound by theory, it is believed the entD knockout prevents premature non-specific activation of the INDS to the “holo” form, thereby allowing higher protein expression. Production of apo-INDS may be performed under standard protein expression conditions in E. coli, e.g., using a chemically defined culture medium and IPTG induction. In parallel, a second strain (Strain B) is cultured to produce a PPTase and PPK. This second strain allows for activation of apo-INDS to the holo form by the PPTase, and regeneration of ATP from inorganic polyphosphate and ADP during indigoidine synthesis by PPK. A defined ratio of Strain A and Strain B cells, e.g., about 50:1 to about 1:1, or about 30:1 to about 10:1, or about 20:1, may be pre-mixed in a defined buffer at about pH 4 to about pH 10, or about pH 4.5 to about pH 9.5, or about pH 5 to about pH 9, or about pH 5.5 to about pH 8.5, or about pH 6 to about pH 8, or about pH 6.5 to about pH 7.5, or about pH 5, or about pH 6, or about pH 7, or about pH 8. The cells are then homogenized and lysed (e.g., using mechanical disruption such as high pressure homogenization or sonication). The resulting mixture may be centrifuged, and the supernatant decanted as the reaction mix. Activation of the apo-INDS may be initiated with addition of coenzyme A (CoA), e.g., at about 1 to about 50 molar equivalents (m.e.), or about 3 to about 40 m.e., or about 5 to about 35 m.e., or about 8 to about 32 m.e., or about 10 to about 30 m.e., or about 12 to about 27 m.e., or about 15 to about 25 m.e., or about 18 to about 22 m.e., or about 20 m.e., to the INDS protein. The CoA and INDS may be incubated for about 5 minutes to about 10 hours, or about 15 minutes to about 8 hours, or about 30 minutes to about 6 hours, or about 45 minutes to about 4 hours, or about 1 hour to about 3 hours, or about 1.5 hours to about 2.5 hours, or about 30 minutes, about 1 hour, about 2 hours, or about 3 hours. After incubation, MgCl₂, ATP, and polyphosphate (e.g., sodium polyphosphate) may be added to the reaction mix. In some embodiments, about 1 to about 10 m.e. MgCl₂, or about 2 to about 8 m.e. MgCl₂, or about 3 to about 7 m.e. MgCl₂, or about 4 to about 6 m.e. MgCl₂, or about 5 m.e. MgCl₂ is added. In some embodiments, the m.e. of MgCl₂ added is relative to the amount of INDS protein. In some embodiments, about 1 to about 10 m.e. ATP, or about 2 to about 8 m.e. ATP, or about 3 to about 7 m.e. ATP, or about 4 to about 6 m.e. ATP, or about 5 m.e. ATP is added. In some embodiments, about 1 to about 10 m.e. polyphosphate, or about 2 to about 8 m.e. polyphosphate, or about 3 to about 7 m.e. polyphosphate, or about 4 to about 6 m.e. polyphosphate, or about 5 m.e. polyphosphate is added. In some embodiments, the m.e. of ATP and polyphosphate added is relative to the expected amount of indigoidine. Indigoidine synthesis may be initiated with glutamine. In some embodiments, the glutamine is added to the reaction mixture at about 0.1 to about 10 mM/hr, or about 0.2 to about 8 mM/hr, or about 0.3 to about 6 mM/hr, or about 0.4 to about 4 mM/hr, or about 0.5 to about 5 mM/hr, or about 0.6 to about 4 mM/hr, or about 0.7 to about 2 mM/hr, or about 0.8 to about 1.5 mM/hr, or about 0.9 to about 1.2 mM/hr, or about 1 mM/hr. After addition of glutamine, the reaction mix is incubated at low temperature to promote precipitation of indigoidine. The reaction mix is centrifuged, and the indigoidine is contained in the pellet. Further purification may be performed to yield high purity indigoidine.

In some embodiments, the amount of indigoidine produced by the cell-free method described herein is quantified by an analytical quantification method, e.g., any of the methods described herein, including but not limited to high performance liquid chromatography (HPLC), gas chromatography (GC), quantitative mass spectrometry, spectrophotometry, or combinations thereof. In some embodiments, the cell-free method described herein yields greater than 50%, greater than 60%, greater than 70%, greater than 80%, or greater than 90% indigoidine relative to the amount of byproducts produced in the method. In some embodiments, the cell-free method described herein yields about 50% to about 100%, or about 60% to about 99%, or about 70% to about 97%, or about 75% to about 95%, or about 80% to about 92%, or about 85% to about 90% indigoidine relative to the amount of byproducts produced in the method.

### Compositions

In some embodiments, the present disclosure provides a composition comprising the indigoidine made by a method described herein. In some embodiments, the indigoidine is made by a heterologous host cell described herein. In some embodiments, the indigoidine is isolated from the host cell, cell extract, culture medium, or whole culture as described herein. In some embodiments, the indigoidine is made by the cell-free method described herein.

In some embodiments, the composition is a dye composition. The dye may be used, e.g., in fabrics such as leather, denim, cotton, linen, and the like; food; beverages; cosmetics; and paper products. In some embodiments, the composition is a bioelectronic composition, in which the indigoidine acts as an organic semiconductor. In some embodiments, the composition is an antioxidant composition. In some embodiments, the composition is an antimicrobial composition.

In some embodiments, the present disclosure provides a composition comprising (a) an INDS described herein; and (b) indigoidine. In some embodiments, the INDS comprises at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5.

In some embodiments, the present disclosure provides a composition comprising (a) an INDS, e.g., comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 1-5; and (b) one or more of: (i) a PPTase, e.g., comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 9-21; (ii) an MLP, e.g., comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 22-24; and (iii) an indigoidine transporter protein, e.g., comprising at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to any one of SEQ ID NOs: 25-32. In some embodiments, the composition comprises any combination of INDS and PPTase as shown in Table 1. In some embodiments, the composition any combination of INDS, PPTase, and MLP as shown in Table 2. In some embodiments, the composition comprises any combination of INDS and PPTase as shown in Table 1, and an indigoidine transporter protein of any one of SEQ ID NOs: 25-32 or a combination of indigoidine transporter proteins as shown in Table 3. In some embodiments, the composition comprises any combination of INDS, PPTase, and MLP as shown in Table 2, and an indigoidine transporter protein of any one of SEQ ID NOs: 25-32 or a combination of indigoidine transporter proteins as shown in Table 3.

All references cited herein, including patents, patent applications, papers, textbooks and the like, and the references cited therein, to the extent that they are not already, are hereby incorporated herein by reference in their entirety.

### EXAMPLES

### Example 1. Small-Scale Production of Indigoidine in E coli Engineered with Indigoidine Pathway

An E coli strain derived from BL21 (DE3) was transformed with a plasmid derived from pTWIST (TWIST Bioscience) that expresses an INDS of SEQ ID NO: 1 under a constitutive promoter, to produce indigoidine. The E. coli strain expresses a heterologous PPTase under an IPTG-inducible promoter on the chromosome. Cells of OD ˜1 were cultured in a 24-well plate at 30° C. for about 20 hours with a shaking speed of 600 RPM in minimal medium supplied with IPTG, kanamycin, glutamate, trace elements, biotin, and manganese. Cell cultures were centrifuged for 10 minutes, and the pellets were extracted with DMSO to be analyzed for indigoidine by HPLC (High Performance Liquid Chromatography). The strain was able to produce 1.4 g/L indigoidine.

### Example 2. Fed-Batch Fermentation Production of Indigoidine in E coli Engineered with Indigoidine Pathway

Preseed culture of the engineered E coli strain described in Example 1, i.e., expressing a heterologous PPTase under an IPTG-inducible promoter on its chromosome and an INDS of SEQ ID NO: 1 on a plasmid, was obtained by inoculating cells of OD ˜0.05 into a 500 mL shaker flask containing 50 mL LB medium supplemented with 10 g/L glucose and 50 μg/mL kanamycin, cultivated on a rotating shaker at 250 RPM and 30° C. for 7 hours. The preseed culture was transferred into a seed medium of 250 mL LB medium supplemented with 10 g/L glucose and 50 μg/mL kanamycin to an initial OD of 0.002, and cultivated for 18.5 hours. The seed culture was then transferred (10% inoculation v/v) to a 5 L bioreactor (Biostat Satorius) with 2 L of minimal medium supplemented with 20 g/L glucose, 50 μg/mL kanamycin, Casamino acids, trace metals, biotin, ferrous sulfate, and antifoam 204. When OD reached ˜5, 0.2 mM IPTG was added to the culture to induce the expression of PPTase. The pH was maintained at 6.8 by using 8% ammonium hydroxide solution. The dissolved oxygen level in the medium was maintained at 10% by automatically supplying a mixture of air and pure oxygen at the rate of 0.3 L/min and by automatically controlling the agitation speed from 300 to 1000 RPM. The temperature was maintained at 30° C. The feed solution was composed of 400 g/L glucose, 44 g/L ammonium phosphate diabasic, 40 g/L glutamine, and 45.6 g/L monosodium glutamate. When the glucose concentration decreased to below 10 g/L, 100 ml of feeding solution was intermittently added. The strain was able to produce 6.8 g/L indigoidine.

### Example 3. Cell-Free Method for Indigoidine Production

FIG. 3 illustrates a cell-free method for indigoidine production. INDS protein is produced in the “apo” form in an E. coli strain (Strain A) with a knockout of the endogenous PPTase entD. Production of apo-INDS is performed under standard protein expression conditions in E. coli using a chemically defined culture medium and IPTG induction. In parallel, another strain (Strain B) is cultured to produce a PPTase and PPK. A defined ratio of Strain A and Strain B cells, e.g., about 20:1, is pre-mixed in a defined 20 mM buffer at pH 5-9, homogenized, and lysed (e.g., using mechanical disruption such as high pressure homogenization or sonication). The resulting mixture is centrifuged, and the supernatant is decanted as the reaction mix. Activation of the apo-INDS is initiated with addition of 20 molar equivalents (m.e.) coenzyme A relative to the INDS protein. After incubation, 5 m.e. MgCl₂ (relative to the INDS protein), 5 m.e. ATP (relative to the expected amount of indigoidine), and 500 m.e. polyphosphate (relative to the expected amount of indigoidine) are added to the reaction mix. Indigoidine synthesis is initiated with glutamine infused at a rate of about 1 mM/hr. After addition of glutamine, the reaction mix is incubated at low temperature to promote precipitation of indigoidine. The reaction mix is centrifuged, and the indigoidine is contained in the pellet. Further purification is performed to yield high purity indigoidine.

### Example 4. Small-Scale Production of Indigoidine in Engineered E. coli K-12 Strain

An E. coli strain derived from K-12 MG1655 was engineered to express chromosomally integrated copies of an INDS of SEQ ID NO: 1 under a constitutive promoter, a heterologous PPTase of SEQ ID NO:9 under a constitutive promoter, and an indigoidine transporter protein under a constitutive promoter, to produce indigoidine. The engineered E. coli strain was cultured in a 24-well plate at 30° C. for about 20 hours with a shaking speed of 600 RPM in LB media. The culture was transferred to a minimal medium supplied with glutamate, trace elements, and biotin, and shaken for another 18 hours. The produced indigoidine was solubilized with DMSO to be analyzed by HPLC. The strain was able to produce 2 g/L indigoidine.

### Example 5. Fed-Batch Fermentation Production of Indigoidine in Engineered E. coli K-12 Strain

Preseed culture of the engineered E coli strain described in Example 4, i.e., expressing chromosomally integrated copies of INDS of SEQ ID NO: 1, heterologous PPTase, and heterologous indigoidine transporter protein, was obtained by inoculating cells of OD ˜0.025 into a 250 mL shaker flask containing 50 mL LB medium supplemented with 10 g/L glucose, cultivated on a rotating shaker at 250 RPM and 30° C. for 6 hours. The preseed culture was transferred into a seed medium of 250 mL of minimal medium supplemented with 15 g/L glucose, trace metals, biotin, monosodium glutamate, to an initial OD of 0.02, and cultivated for 16 hours.

The seed culture was then transferred (10% inoculation v/v) to a 5 L bioreactor (Satorius Biostat B) with 2.5 L of minimal medium supplemented with 10 g/L glucose, trace metals, biotin, monosodium glutamate, and antifoam 204. The pH was maintained at 6.8 by using 8% ammonium hydroxide solution. The dissolved oxygen level in the medium was maintained at 10% by automatically supplying air at a rate of 1 vvm and by automatically controlling the agitation speed from 300 to 1500 RPM. The temperature was maintained at 30° C. Glucose and ammonium phosphate solution was fed to the bioreactor to maintain residual glucose of 0-5 g/L. The strain produced at least 3-fold to at least 10-fold higher titer of indigoidine as compared to the E. coli strain of Example 2.

### Example 6. Production of Indigoidine in E. coli with Engineered Indigoidine Transporter

Engineered E. coli as described in the above Examples (i.e., expressing an INDS and PPTase) were further modified to express one or more indigoidine transporter proteins and tested for indigoidine production.

Experiment 1: The following indigoidine transporter proteins were introduced into the engineered E. coli: (A) proteins of SEQ ID NOs: 27 and 28; (B) protein of SEQ ID NO:26; (C) proteins of SEQ ID NOs: 29, 31, and 32; (D) protein of SEQ ID NO:25; and (E) protein of SEQ ID NO: 30. Indigoidine production and cell growth were measured and compared against a control strain without indigoidine transporter protein. The results showed that all of (A)-(E) had increased indigoidine production as compared to the control strain.

Experiment 2: A combination of the indigoidine transporter proteins of SEQ ID NO:25 and SEQ ID NO:26 were introduced into engineered E. coli and tested for indigoidine production and cell growth as compared to strains expressing one indigoidine transporter protein (SEQ ID NO:25 or SEQ ID NO: 26). All three conditions (either protein alone or in combination) resulted in indigoidine production.

Experiment 3: The engineered E. coli was further modified to have both the indigoidine transporter protein of SEQ ID NO:26 and the INDS of SEQ ID NO: 1 integrated into its chromosome: (A) integration of SEQ ID NO: 1; (B) integration of SEQ ID NO:26; (C) integration of both SEQ ID NO: 26 and SEQ ID NO:1. The results showed that all of (A)-(C) produced indigoidine

SEQUENCES

| | |
|---|---|
| SEQ ID | MNETKREFPTDKCLPDLLWEQARARPESVAVVHEDEILTYRALAERSSELAVYLQHLGVALDDCVGMFVEPSIDL |
| NO: 1 | MVGAWGILSSGGAYLPLSPEYPEERLRYMIEDSRTKVIFSQEGLETRLAELAPRGTRIVTLNDAAEFARAHTKLGK |
| | REPDTGPRPNNLAYIIYTSGSTGKPKGVMIEHRSIVNQMHWMKTVYKLNGEKVVLQKTPMSFDAAQWEILSPS |
| | CGSRVIMGGPGVYRDPGRLIETICRNEVTTLQCVPTLLQALLDTEELHRCESLTQIFSGGEALSRNLALQCLEAMP |
| | RCELVNLYGPTECTINSSAFTVDRTTVKNGPNTISIGMPVHNTQYYILDQHRAPVAVGEIGELFIGGVQLARGYLH |
| | RPDLTADRFIDNPFSTDPRHTRLYKTGDLAFWNADGSVQFAGRADNQVKLRGYRVELDEIRVAIETHDWVKNA |
| | AVIVKNDPRTGFQNLLSFIELNPKEAALMDQGNHGAHHQSKQSKLQVKAQLANMGCRDSAELIGKKVVDLPG |
| | KTPTQEQRRLVFARKTYRFFEGGDIKKEDILRLLGEQVTGTSSRGLEALSFADLGELLRYFGQYLSEERLLPKYGYAS |
| | PGALCATQMYFELNQIGGLKPGYYYYHPVHHQLILIREKAGRATAQVKLHFMGKKRAIQPVYKNNIQEVLEIEAG |
| | HMVGLFEKVLPRYGLGIRELEYTPSTRDNLECADEDYYLGTFELVPYAHAGARPDDSLELYVQAHPGRIADLPAG |
| | QYQYKDGGLEKISDELVLKKHVIAINQRVYENASFGITVISKARKDWMRYVELGRKLQHLQMNDLQFGFMSSGY |
| | SSKSGNDLPSAKRMENILKACGREAGPSYFFVGGRVSQEQMLSEGMKEDSVHMKGPAELIKDDLVNFLPDYM |
| | VPNRVIILDKMPLTANGKIDFKALEKTNVELVERPFVAPRTAVEERISVLWKKEMKRDSVSVQDDFFESGGNSLIA |
| | VALINKLNKEFRTSLPLQVVFECPTIEKLALKIEGGNADPSSRLVRLQAEGTRKPIYCWPGLGGYTMNLRLLANKM |
| | GTERPFYGVQAHGINKDETPYPTIKEMAAEDIKLIKRLQPVGPYTLWGYSFGARVAFESAYQLEQSGERVEHLFLI |
| | APGMPKVRAQDESLHGSAPTYENKAYVTILYSVFAGSITGPALDECLKVAKDEESFASFISGRFENLDPDLVKRIIKI |
| | VAQTYAFKYEFRELAERKINAPITIFKAHGDDYSFIENSSGYSAKAPAVMDLEADHYGLLKEPSIGELLKMIRY |
| SEQ ID | MKMLANNITQCDLITDACLKEDAITLMDMLENQLNHQADGYVVIDKEQSLNYADFYLKVKEIGYCLSEMSSKNS |
| NO: 2 | VGIGLFCDPSIDLICGAWGILSANKAYLPLSPDYPVERLKYMIEDSGIDVIFTQSHLKEQLQDIAPKSVLIITPEDVAL |
| | TIETRTIEDILNTARVPNSTSLAYIIYTSGSTGKPKGVMIEHHSIVNQMRFLAKAFSLGKHSRILQKTPMSFDAAQW |
| | EILAPAIGSQVIIGPLGCYRDPDAIIKTILQHQVTTLQCVPTLLQALLDNPNFLDCLSLTQVFSGGEALTTKLAAQFL |
| | NSFTHCELVNLYGPTECTINSSYFRVTNETLPSYQTSISIGIPVDNTEYYVLDENRLPVAVGEIGELYISGVQLARGYL |
| | HKPEMTKDKFICNHLALETKHPWLYRTGDLVTRGDDGNTYFVGRVDSQVKLRGYRIELDEIRHAIEEHSWIKTAA |
| | MLIKKDARTGFQNLIACVELDEKEAALMDQGNSSSHHKSKTNKLQVKAQLSNSGCRSEELCENRPVILLPYKEGEI |
| | KQREYVFGRKTYRYFEGTEITIETLKTLLTTTQQREICSLPLSHLTLKDFGYVLRYFGQFTSHQRLLPKYAYASPGALY |
| | ATQMYFELHHVFGLDAGIYYYHPVTHELIKISTLSRRQKPMIKVHFIGKREAIEPVYKNNIQEVLKMEAGHMIGLF |
| | DDILPEIGLGIGESEYQAECPDWYDGNIQDYYLGAFEICRYENRLPPFDTDLYLQTHTNKIPEMPCGLYHFSNGEF |
| | VRISDDIVRKKDVIAINQQVYDRSSFGVSIIPRCVPEWHYYITLGRRLHALQSNQLCIGLMSSGYSSESNNDLPSAK |
| | RMRSILNALDRPMAAFYFCIGGCVSQEQYISEGMKEDVVHMKGPVEIIKDDLHQQLPQYMIPNKVLVFDKLPLT |
| | ANGKVDYQSLSKSKAVEDISTQRPLAPLRTETELRLGKIWMEVLKWDSVSAHDDFFESGGNSLMAVAMVNKIN |
| | EAFNIHFPLQILFQSPNIEELAKWIEQADAKTISRLILLNQASQDPIYCWPGLGGYPMSLRLLANKVVPDRAFYGIQ |
| | AYGINESEIPFSSIQRMAEEDIKEIKKIQPEGPYTLWGYSFGARVAFEAAYQLEQAGEEVKALNLLAPGSPHLHVN |
| | QEKYHDKGAEFTNPVFVQILFSVFARSISSPMVKTCLEQVNSEATFINFICSRFKNLDPSLVKRIVRIVTLTYDFKYSF |
| | DEIYHRYLKAPITIFKANKDNDSFIEKSGGFLSTPSKIIELISDHYQLLENEGVTEIEKITSFLNYQ |
| SEQ ID | MNANESFDSHDEVESLPVMLKAAVAADPGKAAVVAENTMMTYSQLWARATAQAEHLRRVGVRPGDYVGLY |
| NO: 3 | LEPGERLLVAVWAVLLADAAYVPLAVDYPSERIRYMIAQSAVAHIITDDVSASPARKLTPDGVHVHIAGEGTPTL |
| | AGAYDPDMSGSAYVIFTSGSTGKPKGVVIPHSAITHQMRWLAQEMALGQARILLKTPTSFDAAQWELLANAV |
| | GGTVIVGEAGIHRDPSRIRALVDSQHATHLQCVPTLWRALCREPEFARCESLTHVFSGGEALTPGDAKDIMQTLP |
| | HARLFNLYGPTETTINATSHRVGVDDVDPPDPVVTIGRPIPGCSVHILDAKRQPVALGDHGELAIGGPQVASGYL |
| | NDAARTEERFVTIEREGVATRVYLTGDIVSRDSAGALHFHGRTDDQVKVNGHRVETEEVRLAVEQHHWVRSAA |
| | VVPWKDPRDGVARLAAFVELDPHEAALMDADRAGQHHRSKKGHEQVLAQLAVLSRDGDRPDDVVLANAEG |
| | TPEQRRFAFGRKTYRFYEGGQLTVEDITRAVDTSQKPVPVAPLAALDVHVLAELLRWFGPFTSPERLLPKYAYASP |
| | GALNATTIYLEACGIEGLLDGMYEYRHASHGLRRVGPVESELPGPLRFHFVGSRRAIDSVYATNIDEVLHFEGGH |
| | MAGLLDHAAAQIGYSAQLQPSTQDARIVAAEHVCTAVIDLGPAAPAGDDLPVRVTVQMHDSVKDARSGTYDV |
| | GPAGLRRLSADIIERRHVIAINQETYDRSSFGVGLSVPSTAGWRGFVALGRALQRLQMNSEGIGLMSAGYSSLTG |
| | RDLPSALQYANIVGDASIMYFAVAGPVSEEQIRSTGMHEDSVHTRGPEEILRDDLRRTLPYYMVPSRITIVDEIPV |
| | SSSGKHDRAALVSVVEASVAPHAVVTPPKTDLERSLLDLWNETLRSDHRSVTADFFDLGGNSLDAVQLINRINRV |
| | LGVLLPVQTVFERPTVRGLAEVLSSRSLAMSRLIKLADGPGPVCVVWPGLGGYPMNLRAFAEHLSSKFTVYAVQ |
| | THGLNVGEVPYTTLREMIDRDIMLIDGALGDQDLAIAGYSFGARVATEVAATLTARGRAIKQVILLAPGSPFVPGL |
| | PLSSGDGFGDSYFRHMAYSVFVGRLPDDEAAAQLASMTDQESFLEALTLWRPGFDLELAARILAVASTTFGFRG |
| | DTVTDVDGLLAKMHVFAALGDTPSFLRSNEVPLEEHGQLRRLSVDHYAILRDPAVETVVGMLEARQVRVG |
| SEQ ID | MTLLGMFSEQISQNPDKIAVTDCNGSLSYLDFYLAVMNTGKYLADIQVSNENCIGLYCEPSIDMVCGAWGILAS |
| NO: 4 | GNAYLPLAPEYPSERIRYMIQDSKVRVIYTQEHLKAQLEAVVPEHVTVVSGADIPLSFSDGTASLSNTVCSKITPEN |
| | LAYVIYTSGSTGKPKGVMIQHRNIAQQMAFLKQCFSFGEHTRILQKTPMSFDAAQWEILAPVFGGHLFVGPSGC |
| | YRDPDVMVEALLKYDISVLQCVPTLLQALIDHPLFVDCKELKQVFSGGETLTRQLSKEFYGVRPESELINLYGPTEC |
| | TINSSYFRVCSEELDNYPSAISIGKPVVQTQYHILRNDGQPTLVGEIGELYISGPQVAKGYLHRPDLTEDKFVPNHIS |
| | KDPGHSRLYRTGDLAHCDGEGNVHFAGRADNQVKLRGYRVELDEIRHAIEKHLWIKNAAMVINNDARTGSQN |
| | LIACVELDKTQAAIMDQGNHGAHHQSKANKHQVKAQLSNAGCRQSEECVGKLKIELKGKDATDRQKEKAFGR |
| | KTYRFFDRHHPVTKEELTVLLTFEQPKSLSCDVAQLTESQFGELLRHFGQYISDERLLPKYAYASPGALYAAQMYL |
| | ELNGLFGWPAGIYYYHPIEHCLIKIKTLVQSEVPVFKLHFIGKRDAIEPVYKNNILEVLEMETGHILGLFDELLPNLGL |
| | SIGRHHKLEQLPSWYDGKKHDYNLGTFEICSNESAPEIEPPELYIQVHSNRVLGLTEGLYRFVNQDFEYLSDQLIM |
| | KRDVIAINQEVYDRASFGISMVENTSEQAMRYISLGRTLHKIQSNPLLLGVMSSGYSSKTGNDLPSAIRMRQILNT |
| | QGKNLKAFYFCIGGGISEEQYFSTGMKEDTVHMRGPTEIIRDDLMSQLPQYMIPNKVVIIDKLPQTANGKVAYQ |
| | ALKALDVVVNCGSEKKFVPLVTETEKQLGEIWCRIMKWNTASAEDDFFECGGNSLTAVAMINRINQTFEIKVPL |
| | QVLFKSPTIKQLAKWIDSQDEHTQSVSRLIELNNAQQRPVFCWPGLGGYPMNLKLLANQVSPERQFFGVQALG |
| | INEGEIPLSNVQEMAKADIELIKAVQPDGPYTLWGYSFGARVAFETAYQLEQMGETVEALNLIAPGSPQTHCDLE |
| | QMNQGEASFSNPVFVTILFSVFAHTIDGALLQSCLNQCHDEDSFVKFMCKRFPVLQEDLIRRITRIVQVSYNFSYT |
| | FEELVGRKINTPVTIFKAQQDNYSFIESAPAFSKVPPKIVNLQVDHYQVLKEQGVAELK |
| SEQ ID | MTSVPAPKTLTEMLRYQVAAHPQAVASIDADGKLTFEELLSRADFAAAALTAAGVGPRDTIGLFMAPSNHLLIA |
| NO: 5 | TWAILSADASYLPLAVDYPAERISYMISDSKIRHVVVDGGSYELIEHLVPEHVQVLRIEQLFQAGPKRDYSAYTEDL |
| | PAYVIYTSGSTGRPKGVVIGHPAIVNQMQFLAESAGLNPGERILLKTPISFDAAQWELLANAVGATIVVGPHGVH |
| | RDAELISNLVLEYEVSLLQAVPTLWTALLETGKLTRCRSLRGLFSGGEVLPSRLARELLATLPQAQLVNLYGPTETTI |
| | NATWLRLSIGDIPDTPAVSIGSAVPGCQVHILDAELRPVLDGQTGELCISGSQLAYGYLHRPRLTAERFVEADVDG |
| | RPVRLYRSGDVARIDASGQVEFCGRIDDQVKVNGHRVETDEVRLAIEEHHWVRQAAVVPWRSPEDATIRLGAF |
| | LELDPAEATLMDQDAAGEHHRSKASHVQVRAQLAGLGVRNFTHPQRSVVLPGREGTPEQFQAAFARKTYRFF |
| | EGLPISAADLVALGESLDQPWAIGPRSGLVTISDVADVLRWMGPFPSSQRLLPKYAYASPGALNATQIYLETSGM |
| | PGLDAGMYYYHPTKHELIHVGHQAARLSAPSLRLHLAGLPRVIESVYSTNVVEVLHMEAGHMVGVLDRAAAAR |
| | GLRLQPVPSVDLPELGLTEKWESTGVFDLIECVDAVPSAPLVDLTFQIHGKVAGARHGTYCLKDGTLHRLSNHVIE |
| | RRQVIAINQGTYDRSSFGVTLSCRREQGWSGFFELARALQKLEMNEQGIGLMSSGYASLSGRELPSARRYDQIA |
| | RAWGQDESRLSYFAVGGSVSAEQVASTGMKEDAVHLRGPEEIVKDDLRRILPHYMVPSRVEVLDKLPMSSSGK |
| | VDRAALIQQLESDQRPQRAVILPSSKLETDCLNVWSRMLDREVLSVSDDFFDIGGNSLTAVKLINALNEHFGSSLP |
| | IQSIFEAPTARELAERIRQVAPANASRLVLLAPGTGEPVFVWPGLGGYPMSLRGLAARISHGRPVYGIQAYGLNP |
| | GESPCTTMEEMVAADVEPVLEAAEGNSVTLMGYSFGARVAAEVALWLEARGVTVDRLTLIAPGSPEIDGVPDR |
| | PWNGPNAFLDPYFRMILMSVFTGSLDGLHRDALLAKVRTRNEFMELLAGRVPALDMALASRIIEVVAATYSFRS |
| | QPASDIQQVLSQTQVLIAAGDGPSFASPYRDRMEARGAVVELEANHYQVLHEPTVDLTANFIMALQEQPA |
| SEQ ID | MDNISNHAFNYPVLVLNKGLLPEHDDIALARYLFSALALAVSRITQNEEMIVGFHLHPQEITRWKDDECIRQYILP |
| NO: 6 | LNIRFNSATPIAGFIREIMTWMTPDAIHQKNAMGASVLTLGPQHALHDIFDLEISWQPPVESEPVQALTCHVAS |
| | REDALVLTLRFNPARFSATQMQKLPEVWRQITASAAKNGAETLRDIGLIDDAERQRVLHAFNQTEQAWDGETT |
| | VAARLKNRAQRHPEQTAVVFRDETLSYRQLYQQAGALAHYLNALETERERCVGLFVEPSLTLMTGVWGILLSGN |
| | AYLPLSPEYPEDRLAYMLENSQTRIIVTQPHLRERLLALAPPGIQVVTSDDVDAFMRQHAHSLPDAPQNDIAPHH |
| | LAYVIYTSGSTGKPKGVMIEHHSVLNQMNWLAQTVGLNQETVILQKTPMSFDAAQWEILSPACGCRVVMGEP |
| | GVYRNPEQLVDMLAEYRVTTLQCVPTLLQALLDTERLTHCPALRQIFSGGEALQKHLAQACLETLPDCELINLYGP |
| | TECTINNSAFRVDPVSVRQGPDTLSIGAPVANTRYYILDNCLTPVAVGQIGELYIGGDGVARGYLNRDDLTAERFI |
| | VDPFAPAGSGRRLYQTGDIASWNPDGTVQYAGRADNQVKLRGYRVELDEIRSAIETHEWVKAAAVIVRNDPFT |
| | GYQNLISFIELNAREAALMDQGNHGSHHQSKADKAQVMLQLANKGCREFPAASQPYTLDLPGKQPDEKQRLT |
| | AFSRKTYRFYDGGAVSREDILSLLHEPLLTAISRQPDALTLDELGHWLRYLGQFTSAERLLPKYTYASPGALYATQV |
| | FLELNGVAGLTAGHYYYQPVHHQLVRVSEQAAVTPGSLRLHFVGKKSAIEPIYKNNIREVLQMEMGHIIGMLDII |
| | LPDYGLGVALCDAAALDPTPLAIDLDDDYLGACDVLSGPRLPTDDDLDIYVQTAGANIADLPVGTYRYVRGDLQ |
| | HIADDVIDKKHVIAINQAVYERSSFGISVASRTEGWAGYVHVGRKLQRLQMNPLNIGLMSSGYSSETGNDLPAA |
| | RRFWQILGHRTGPYYFFIGGRISDEQKYSEGMREDAVHMKGPAEMIRDDLAAFMPDYMMPNKVLILDEMPLT |
| | ANGKIDMKALANINVELKHKTIVAPRNPLEHQVMAIWQAKLKREEMSVDDNFFESGGNSLIAVSLINELNATLN |
| | ASLPLQVLFQAPTVEKLAAWLSRARREPVSRLVQLQPKGRQAPIYCWPGLGGYCMNLRLLARQLGAERPFFGIQ |
| | AHGINPDETPYATIGEMAARDIELIRQHQPHGPYTLWGYSFGARVAFETAWQLELAGEVVENLYLLAPGSPKLR |
| | DERVAAMNRKADFDNPGYLTILFSVFIGSITDPELERCLETVRDEESFVAFITGLNPALDDGLVRRITRIVAQTFEFT |
| | YTFSELQQRQLNAPVTIIKAQGDDYSFIENHGGFSAQPPTVLELMADHYSMLKAPGIDELTSVIQYQQSPPSLVG |
| SEQ ID | MLENNITQCDSINDVYLKEEAITLMDMLESQLKHQADGYVVIDQEESLSYADFYLRVKEIGYCLSEISSKNSVGIGL |
| NO: 7 | FCDPSIDLICGAWGILSADKAYLPLSPDYPTERLKYMIEDSGIDVIFTQSHLKAQLQDIAPKSVLIMTPEDVALTIKT |
| | RTIEDILGTVQVPKPTSLAYIIYTSGSTGKPKGVMIEHHSIVNQMRFLAKAFKLGCHSRILQKTPMSFDAAQWEIL |
| | APAIGGQVIMGPLGCYRDPDAIIKTILQHQVTTLQCVPTLLQALLDNPNFLDCLSLTQVFSGGEALTTKLATQFLN |
| | SFTHCELINLYGPTECTINSSFFRVTNETLPNYQTSISIGAPVDNTEYYVLDDDRLPVAVGEIGELYISGAQLARGYL |
| | HKPEMTKDKFICNHLVSGTQHQWLYRTGDLVTRGADGNTYFVGRVDSQVKLRGYRIELDEIRHAIEEHSWIKTA |
| | AMLIKKDARTGFQNLIACVELDEKEAALMDQGNSSSHHKSKADKLQVKAQLSNSGCRSEELCENRPTFLLPYQE |
| | GEIKQREYAFGRKTYRYFEGTEITVEKLKKLLTATQSNEISSLPLSHLTLNDFGYALRYFGQFTSHQRLLPKYAYASP |
| | GALYATQMYFELHNVLGLDAGIYYYHPVTHKLIKISTLSRRQMPTIKVHFIGKHEAIEPVYKNNIQEVLEMEAGH |
| | MMGLFDDVLPEIGLSIGKSEYQDECPDWYDGDIQDYYLGAFEICSYEHGLPPFETDIYLQTHAHKIPEMPCGLYH |
| | FSNGEFVRISDDIVRKKDVIAINQQVYDRSSFGVSIIPRCVPEWHYYITLGRRLHALQSNPLYIGLMSSGYSSKSNN |
| | DLPSAKRMRSILNALDRPMAAFYFCIGGGISQAQYMCEGMKEDVVHMKGPVEIIKDDLQQQLPQYMIPNKVL |
| | VFDKLPLTANGKVDYQSLSESKAVENVSTQRLLVPLHTDTEIRLGKIWMEVLKWDSVSALDDFFESGGNSLMAV |
| | AMVNKINAAFNIRFPLQILFQSPNIAELAKWIEQTDSKTISRLILLNQASKDPIYCWPGLGGYPMSLRLLANKVVP |
| | DRAFYGIQAYGINESEIPFSSIQRMAEEDIKEIKKIQPEGPYILWGYSFGARVAFEVAYQLEQAGEEVNALNLLAPG |
| | SPHLDMKQAEYMDKGAEFTNPAFVKILFSVFSRSINSPMVKTCLEQVNSETTFINFICSRFKNLEPSLVKRIVRIVTL |
| | TYDFKYSIDELYHRHLKAPITIFKANRDNDSFIEESDVISSMSPKIIELISDHYQLLESEGVAEIEKII |
| SEQ ID | MSAQMNTLQAFLQQHIQSSYTHTSPYLRTLPAMLLAQVHSRKNEIAIADQETEYSYETTLLHAVGIAAALRAKGV |
| NO: 8 | SYNDCLGLFIDASADLALATWGILFAGAAYLPLATDYPQDRLSYMVSDARVKLVLTNNRSRERLAGVLLPGVSLL |
| | NIDDVAPASITEVDQVLTELLEQDGEDLAYVIYTSGTTGKPKGVAISQQAIANQLAWLTHEGYLLPGHRILQKTPV |
| | SFDAAQWELLGMCCGARVVMGQPGVYRDPEALIRQVQQHGITTLQGVPTLLQALSELPAFADCDTLHSLFSGG |
| | EGLSRKLASKLLQILPDCRLVNLYGPTECTINATHYRIDQRSLDQEWEIAPIGLPVAGLHYQVLDLQLAPVAAGQT |
| | GELFIGGAQLANGYLFRAEQTAEKFLSLPLDAASPQVRMYRTGDLVRVDPDGVLHFVGRTDNQVKFRGYRIELD |
| | EIRLAIENHDWVKSAAVFIKENARSGHAQLIGAVELNPNEAKLMDQGAAESHHQTKASRHQIKAQLSGRGFRS |
| | DASLAGRQQIALPGQQETAAQRERVFARKTYRFFHGGPVAATDILQLLAAQPEAAPSARLADLDASKLGEMLRY |
| | LGQFSSEERLLPKFGYASPGALYATQVYIELNQVADLPAGYYYYHPLQHRLYLLAASSATEPCLRLHFVGKIPAVRE |
| | VYKNNILEVLEMESGHILGMLDHVLPGYGYGLGLGRHTPEVLARLDCPEEHDYLGSYDIVALAERIDDLAVDIYVQ |
| | AQQGRIHDLADGNYLYRQGRLDKVSGHLIEQRHVIAINQQVYQRASGGLSFVSQSQAPWRQYLDLGRSLQRV |
| | QMNRLGVGTMSSGYSSKSGNNLATALRLNDIVGDAGRESGPSYFCLFGKVSAAQLSHQGMDEDAVHMKGPA |
| | ELIRADLLNSLPDYMVPGRIAIINQMPHTTSGKVDVQALKQCADFQLADQEQPHVAARTATEQAISQIWCHILQ |
| | LEEVSVMDDFFALGGNSIQAVAIARAINRQFAAKVPIQLLFSAPTIEKLAQAVSGGDMQTASRAVALAGHDGTL |
| | ATFCWPGLGGYPMNLRLLGEAVAGERRFYGIQAHGINAGETAFDSIEAMAREDVQLLRSLQPQGPYALWGYSF |
| | GARVAYEVAYQLEQQGETVSQLVLLAPGSPRLSHAEPVSRDERELFANPAFLTILLSVFAHDIDPALNADCLLRVD |
| | SRATFIRFAAEKFPAIDRTLIDAIANVVAVTYSPDYQIALADKPLCCPISVWRARGDGPSFIADGEQAGLRIRWHD |
| | LDVGHYAVLKAEGIAAMQAAVLA |
| SEQ ID | MFSSFIKDIEWLTIYQAGNTAVYPGHCARCHFDLSAYRDELFAVAGLPVPAEIARSVPKRRAEYLAGRYLAQTVLS |
| NO: 9 | RLGVAGYVLTSARDRSPQWPQGIAGSLSHNADSVLCAAHPCNQAMTCVGLDIETRMSAERADNLWPGIADEI |
| | EYDWLHSHDPISFASMLTLSFSAKESLFKALYPQVKRYFDFLDVRMVELDTVQQTFTLQLLIDLSPDYLMGCRFSG |
| | AYQLRESDITTFLEN |
| SEQ ID | MEQTVIHTNQASLPEYDNGFITQIEYGTVVKHPQVHFWHARFDLSYYHDELFEQLNLPFPATLTKAVKKRRAEYL |
| NO: 10 | AARYCARQLLAQLGQPAFNLISGHDRAPIWPQNICGSVSHSSHCAIVLAAPQTHNRLIGVDIEAIVDRQNIDEITK |
| | MIVNDKEIQLLKHCHLPLEQAFTLAFSVKESLYKALYPQVKRFFGFEAAEIIALSLENNEITLALRETLTPHYPAGTLF |
| | RGQFIIYPQEILTLIIQ |
| SEQ ID | MNTLPACCAPLRHHWPLPRPLPGAVLVSCAFDPAHLATDDFQRAGIVPSASLQRSVAKRQAEYLAGRVCARAA |
| NO: 11 | LQHLDGRDYVPGTHEDRSPIWPAGIHGSITHGKGWAAAVVAGENSCQGLGLDQEALLDDERAERLMGEILIPA |
| | ELERLDRRQLGLTVTLTFSLKESLFKTLYPLTRQRFYFEHAEVLDWSAEGLARLRLLTDLSPQWQQGAELQGQFCL |
| | QDGHLLSLVSV |
| SEQ ID | MKIYGIYMDRPLSQEENERFMTFISPEKREKCRRFYHKEDAHRTLLGDVLVRSVISRQYQLDKSDIRFSTQEYGKP |
| NO: 12 | CIPDLPDAHFNISHSGRWVIGAFDSQPIGIDIEKTKPISLEIAKRFFSKTEYSDLLAKDKDEQTDYFYHLWSMKESFI |
| | KQEGKGLSLPLDSFSVRLHQDGQVSIELPDSHSPCYIKTYEVDPGYKMAVCAAHPDFPEDITMVSYEELL |
| SEQ ID | MLDAILPAGAVLAEERGPAGEHPLHPAEAGAVARAVPSRRREFAATRACARTALAALAAFDGDAPGAAPVAIP |
| NO: 13 | KGRGGDPVWPRGVVGSLTHCAGYRAAVVAGVDALRTIGIDAEPHAPLPREARDVVGLAGELDPHPPLGADVH |
| | ADCVLFSAKESVGKAHYARYREWLGFADLHVTLHPGGAFTARRCAPGPVPFPAYRGSWRVAEGLVLTCAWLA |
| | VPRVPSAVPRPA |
| SEQ ID | MTHGFRALLPSAVEVEVATDDADGASPFDAERAAVARAVPVRRAEFFTVRACARRALARLGQPPVAIVPGPGR |
| NO: 14 | EPVWPAGVVGSMTHCRGRRAAAVARSDEILVLGIDVELHAPLPEGVAELVMSPAERGRLDELGRIQPSVAWDR |
| | VVFSAKESVFKAWYPLARSWLDFLECDLELDARTGTFDARLLVPGPLIGLDRLQVLRGRWATEGGFITTAVWEA |
| | AAPTRR |
| SEQ ID | MLKTILPEGLSVVETRADVPESTLYAEEAAVVARAVHKRRAEFTAVRHCARQAMAELGVAPAPLLPGERGAPR |
| NO: 15 | WPDGVTGSMTHCDGYRAAVVGLASRFRSVGVDAEPHDTLPDGVLGTIALPSERERHTALRRDRPDVHWDRLL |
| | FSAKESVYKTWFPLTGRWLDFEEADITLAPGAGHTGTFTARLLVPGHTTSGDPLTGFEGRWTVAEGLVCTAIALP |
| | APVPRP |
| SEQ ID | MIEELLPAAVVAVEAHGDEAAVDGALYPEEQAVIARAVEKRRREFTAVRVCARRAMEKLGVPPQPVLPGERGA |
| NO: 16 | PRWPAGLVGSMTHCEGYCAAVLVRAGELASLGIDAEPHDRLPEGVLSSVALPTEERRLYDLGRSRPDVHWDRLL |
| | FSAKESVYKAWFPLTGKWLDFLEADIEIFTEPAAQGKALSGGFRAELLVPGPLVNGRRLDAFDGRWTVRRGLVA |
| | TAVTVPHH |
| SEQ ID | MIESLLPPEAVWAEHFGPDPSARLLPEEEPHVAQAVARRREEFTTVRACARRAFAALGLPPGPVLPGVRNAPR |
| NO: 17 | WPAGVVGSMTHCDGYRAAVLARATDLAAIGIDAEPDLPLPDGVLEAISLPGERARLGLGGPAAPRPHGGRTVC |
| | RDRLLFSAKEAVYKSWYPLLGTELDFHEADISFHGGPDGTERAGGTERADSTVRTEGTGGVSGTFTARILRPEREP |
| | GGRLVEEFTGRWLSERGILVTAIGVPAPRTVPR |
| SEQ ID | MATESPADSRSVTTPAPPPGVELVWYGRVPALAADALAHRGLLDAGEQARLDGFLRPRDRDAYAVAHVALRRL |
| NO: 18 | LGERLGLPPGAVVVERRPCLHCGGPHGRPVVAGDPVHFSLSHTTGAVLIALARTAVGVDIERLPSPASVDDIADQ |
| | LHPGERAGLAALTGEERVRAFARCWTRKEAFLKATGAGLTEDLSRTLVGAGPRPAEVPGWSIADLAADAGYTA |
| | AVAVQTPQ |
| SEQ ID | MIEELLPGTVVAVEAFGQDDAGHLPLYPEEEELVARAVAKRRREFTVVRSCARRAMEKLGVPAQPVLTGERGAP |
| NO: 19 | RWPEGIAGSMTHCDGYGAAALVRLTDLASLGIDAEPDGPLPDGVLESIALPAEVALLRRLGGARPGVHWDRLLF |
| | SAKESVYKAWYPLTGQWLDFAEADIEIRVDPADPRRGTLHAALLVPGPTVDGRRLSRFDGRWSARDGLVTTAV |
| | TVPRT |
| SEQ ID | MTSEISPVRVLRAGAGPDPSVPPVVDGSVALWLVPVTADPGAYELLDAGERQRSAALLREADRTRYLAAHGGL |
| NO: 20 | RRLLGHYLGTPPDEVVFVREDCPLCGGPHGRPAVRDGGIHFSLSHSEDLVLVGLAGRPVGVDVEAFPAAGVSDL |
| | VADTLHPREREEFARLAPEVRTAAFTRCWVRKEAYLKGTGEGLAGGLERTYVGTGPGPAAVPGWSLTDVPVGH |
| | SYAAAVALSQQ |
| SEQ ID | MSGAVVPAGLLSALRHVLSPPDDAGASVAALWRPWPLPVAGGSVAALSLPALDALLGDIVPASLHPAIPGRCVR |
| NO: 21 | SRQLSFLAGRLCAEQALAALPLTAAVLGRDASGRPLWPAGVTGSITHSPTLAAAVAAWSPQTDGLGVDCEPLAA |
| | GERLDDIVSACCTAADRACLPSRAAGTMATLIFSAKEAGYKALSHRFGRIVDFTEFEVCQLARDAGQLWLAPVP |
| | GSEWHRRIQPFAVQFTFDADSVYTLADLRGIP |
| SEQ ID | MSESQNDERAYRVVRNDEDQYSIWWADRDLPAGWHAEGTEGPKDACLQRIEEVWTDMRPLSLRRRMQQQ |
| NO: 22 | DQALIAI |
| SEQ ID | MSNQDTEDTQKYKVVVNHEEQYSIWPADRENALGWKDAGKEGSKAECLDYIKQVWTDMRPLSLRKKMEEAA |
| NO: 23 | KGS |
| SEQ ID | MTDEREDTTVYKVVVNHEEQYSIWPADRENALGWKDAGKQGLKAECLEYIKEVWTDMRPLSLRKKMEELKS |
| NO: 24 | |
| SEQ ID | MPASRLWRDTLLTAVVPLIWGSTYLVTTQWLPAGLPFSAGVLRVLPAGLLLLLWTRHLPQRQEWPRLLLLSLLNI |
| NO: 25 | GAFQALLFVAAYRLPGGIAAVLGAIQPLLMMGLLWGLERQTPRPLVLLAACAGLAGMAVLLNAGNARWDSIG |
| | VLAAAGGAVVMALGMYLARRWGSSLPLLALTGWQLALGGLMLLPAALWLDPPLPALSAANVAGYAYLSLVGA |
| | LLAYGLWFRGLRLLPPVAVSALGLLSPVAAVILGWVALGQRLHGWTLIGMVTVLASVLCVQLASSRRPARAG |
| SEQ ID | MKLKDFAFYAPCVWGTTYFVTTQFLPADKPLLAALIRALPAGIILILGKNLPPVGWLWRLFVLGALNIGVFFVMLF |
| NO: 26 | FAAYRLPGGVVALVGSLQPLIVILLSFLLLTQPVLKKQMVAAVAGGIGIVLLISLPKAPLNPAGLVASALATMSMAS |
| | GLVLTKKWGRPAGMTMLTFTGWQLFCGGLVILPVQMLTEPLPDLVTLTNLAGYLYLAIPGSLLAYFMWFSGLEA |
| | NSPVIMSLLGFLSPLVALLLGFLFLQQGLSGAQLVGVVFIFSALIIVQDISLFSRRKKVKPLEQSDCVIK |
| SEQ ID | MRGRSTDWLLAMAGGALLALMINYNSLLAKHTTPVFASWVAHGLGAVAAFALVVLYSRLFRSPGKEMEPQRA |
| NO: 27 | KVPLWFYLGGIPGTLTVILAAVTVNSGLSLSGTIALMLLGQVLFGLVSDYFGLFRTPKRRIVATDFLVALCILSGSALI |
| | IFGRAPMIAYILLAFFNGTVIGTSRAINGRLGAEIGSVKASLWNHLIGFLFLTPALLLLGGWKFEVIPEAPASAYIGG |
| | FFGALFVAVNSYVFPRLGAMNASLLVISGQMISAVLLDYRNQGVAPTVTRCLGVVIVLFGVYLTRAAKNPRVKDK |
| | SQ |
| SEQ ID | MIAYILLAFFNGTVIGTSRAINGRLGAEIGSVKASLWNHLIGFLFLTPALLLLGGWKFEVIPEAPASAYIGGFFGALF |
| NO: 28 | VAVNSYVFPRLGAMNASLLVISGQMISAVLLDYRNQGVAPTVTRCLGVVIVLFGVYLTRAAKNPRVKDKSQ |
| SEQ ID | MINKLLNDRSYHIEFNGHLTNHIKHAVIALHGLGISASRIKDYYDNYVKLTPYGMGLEPPKTLKHVVDSSNWKHF |
| NO: 29 | LGKRTSYSSYCDYFEEEIKQKGIEQVLQEYMPTLLSGWAGALTHGTIHLGWALDINHPWMIIEGLAYMAFSYVPC |
| | HSERAFIDSSLNDKNAFDSILRISTLWEQRRAELTDWVNSLIDNEDLADTDLIHPELKRSGLQYRIARILMQGHPEI |
| | YRLPVWIETQNVEESWAQLFYVVTLIYLAKPGDFLFLHLITSLFAMKNIASRLPAEESKNIIKCYWVGMLCILFSTT |
| | DLSKPAKFSALNQTYSFRQDEMTYPVWEQEWSHIIARALEEEEEHNPKLVYVMNQLWKEQGLTIYRAASSQFTS |
| | TPLLPPSFEEAPIE |
| SEQ ID | MTQHTKPAGVLLLLAILLLSLNLRPAIAAIGPLIQSISHDAGVRSVGISLLTTIPVLMMGLGAIYAARIRQALGERGG |
| NO: 30 | ITAGSALIAVACLMRLWADDRNGLFLSAALVGLGIAVIQALLPGFIKRNFGARTSSVIALYSTGIVAGAAIGSGTAS |
| | WLEDQLGWLSTLASWSLPAALATVFWLLASLGSQHEGRSNVQATTRSVPFWRIGRCWSLLLFFGIGTGAFMLA |
| | MAWISPFYIGLGLGKATAGLLLSVLTIVEALTALWLSFAIGRFPDRRGLLLTSLLLVAAGFLLLIVAPLLAPYFAVSLL |
| | GVGIGILFPLSIIVAMDHLKDPTLAGNFTAFVQGGGFIIASLVPLTAGSLRDVFNDLSYIWLLMSIASLGMLVLAVR |
| | FSPASYQQFQQDLAELQAGELKPA |
| SEQ ID | MINTIRNILLAVIGGSLLTLMIYTNSMLSESTTPFFASWVAHGIGAIVALILFIIVSKLFSRKEIDESKHKKSNIPIWFYL |
| NO: 31 | GGIPGAFTVVLAAIAINGGLPLSSTISLGLVGQILFGLVADRFGFLGTRKRKIVIQDFYVIFFVLFGSILILFGGSN |
| SEQ ID | MLSLIFIAFLNGIFIAATRTINGQLSVSIGSFGASLWNHIGGFFLLTVILTLLSSWQWNKWDFSVIPTAAYMGGVF |
| NO: 32 | GALFVAVSSYIFPKLGAMNAAVLVIAGQMLSAVLLDWLYQGMTPGLVKIVGVLFVLIGIYLTRKTTELK |

1. A heterologous host cell capable of expressing an indigoidine synthetase (INDS) comprising: at least 70%, at least 80%, or at least 90% sequence identity to any one of SEQ ID NOs: 1-5, wherein the heterologous host cell is capable of producing indigoidine.

2-4. (canceled)

5. The heterologous host cell of claim 1, wherein the heterologous host cell is further capable of expressing a 4′-phosphopantetheinyl transferase (PPTase), wherein the PPTase is capable of activating the INDS.

6. The heterologous host cell of claim 5, wherein the PPTase is an endogenous or exogenous PPTase.

7. (canceled)

8. The heterologous host cell of claim 6, wherein the PPTase is an exogenous PPTase, and wherein the exogenous PPTase comprises at least 70%, at least 80%, or at least 90% identity to any one of SEQ ID NOs: 9-21.

9-12. (canceled)

13. The heterologous host cell of claim 1, wherein the heterologous host cell is further capable of expressing a nonribosomal peptide synthetase (NRPS) accessory protein.

14. The heterologous host cell of claim 13, wherein the NRPS accessory protein is an MbtH-like protein (MLP).

15. The heterogeneous host cell of claim 14, wherein the MLP comprises at least 70%, at least 80%, at least 90%, or 100% identity to any one of SEQ ID NOs: 22-24.

16. The heterologous host cell of claim 1, wherein the heterologous host cell is further capable of expressing an indigoidine transporter protein.

17. The heterologous host cell of claim 16, wherein the indigoidine transporter protein comprises at least 70%, at least 80%, at least 90%, or 100% identity to any one of SEQ ID NOs: 25-32.

18. The heterologous host cell of claim 1, wherein the heterologous host cell comprises one or more modifications to increase glutamate and/or glutamine production.

19. The heterologous host cell of claim 1, wherein the heterologous host cell comprises one or more modifications selected from:
a) increased levels of one or more of acetyl-coenzyme A synthetase, NADP-specific glutamate dehydrogenase, glutamine synthetase, isocitrate dehydrogenase, dihydrolipoyl dehydrogenase, phosphoenolpyruvate carboxylase, transcriptional repressor IclR, NAD(P) transhydrogenase subunit alpha, NAD(P) transhydrogenase subunit beta, Type III pantothenate kinase, glucose facilitated diffusion protein, glucokinase, or S-formylglutathione hydrolase;
b) decreased levels of one or more of D-lactate dehydrogenase, ubiquinone-dependent pyruvate dehydrogenase, aldehyde-alcohol dehydrogenase, phosphate acetyltransferase, acetate kinase, bifunctional glutamine synthetase adenylyltransferase, glutaminase 1, glutaminase 2, glutamate synthase [NADPH] large chain, glutamate synthase [NADPH] small chain, aerobic respiration control protein, 2-oxoglutarate dehydrogenase E1 component, dihydrolipoyllysine-residue succinyltransferase component of 2-oxoglutarate dehydrogenase complex, malate dehydrogenase, pyruvate kinase I, adenine deaminase, cytidine deaminase, guanine deaminase, outer membrane porin C, respiratory nitrate reductase 1 alpha chain, L-asparaginase 2, 2-iminobutanoate/2-iminopropanoate deaminase, adenosine deaminase, cytosine deaminase, dipeptide-binding protein, periplasmic oligopeptide-binding protein, fused glutamine synthetase deadenylase/glutamine synthetase adenylyltransferase, L-glutamine ABC transporter periplasmic binding protein, L-glutamine ABC transporter membrane subunit, L-glutamine ABC transporter ATP binding subunit, pseudouridine-5′-phosphate glycosidase, pseudouridine kinase, pseudouridine transporter, or tautomerase; and
c) altered expression of one or both of DNA gyrase subunit A and cytochrome d oxidase cydAB.

20. The heterologous host cell of claim 1, wherein the heterologous host cell is bacteria or fungi.

21. The heterologous host cell of claim 20, wherein the heterologous host cell is bacteria, and the bacteria is Escherichia, Corynebacterium, Bacillus, Ralstonia, Zymomonas, or Staphylococcus; or wherein the heterologous host cell is fungi, and the fungi is Pichia, Saccharomyces, Candida, Yarrowia, or Aspergillus.

22-24. (canceled)

25. A method of making indigoidine, comprising:
(A) expressing an indigoidine synthetase (INDS) comprising at least 70% sequence identity to any one of SEQ ID NOs: 1-5 in a heterologous host cell;
or
(B) contacting an indigoidine synthetase (INDS) comprising at least 70% sequence identity to any one of SEQ ID NOs: 1-5 with glutamine under conditions sufficient to produce indigoidine, wherein the method of (B) is a cell-free method.

26-47. (canceled)

48. A composition comprising indigoidine made by the method of claim 25.

49. A nucleic acid encoding an indigoidine synthetase (INDS) comprising at least 70% sequence identity to any one of SEQ ID NOs: 1-5, wherein the nucleic acid is operably linked to a heterologous regulatory element.

50. (canceled)

51. (canceled)

52. A vector comprising the nucleic acid of claim 49.

53. (canceled)

54. (canceled)

55. A host cell comprising the nucleic acid of claim 49.

56-60. (canceled)

61. The method of claim 25, wherein, in the method of (B):
(i) the INDS is an activated INDS comprising a 4′-phosphopantetheine (PPT) group;
(ii) the method further comprises, prior to the contacting, incubating the INDS with a PPTase and coenzyme A to form an activated INDS comprising a PPT group;
(iii) the contacting is in the presence of ATP, MgCl₂, FMN, or combination thereof;
(iv) the contacting is in the presence of an MLP, a polyphosphate kinase (PPK), ADP, inorganic polyphosphate, or combination thereof; or
(v) any combination of (i)-(iv).

62-67. (canceled)

68. The heterologous host cell of claim 1, wherein the INDS comprises: at least 90% sequence identity to any one of SEQ ID NOs: 1-5, and wherein the heterologous host cell is further capable of expressing: (i) a PPTase comprising at least 90% sequence identity to any one of SEQ ID NOs: 9-21; (ii) an MLP comprising at least 90% sequence identity to any one of SEQ ID NOs: 22-24; and (iii) an indigoidine transporter protein comprising at least 90% identity to any one of SEQ ID NOs: 25-32.


SEQUENCE LISTING (32 sequences)

SEQ ID NO: 1 - protein, 1275 aa, Archangium violaceum
MNETKREFPTDKCLPDLLWEQARARPESVAVVHEDEILTYRALAERSSELAVYLQHLGVALDDCVGMFVEPSIDLMVGAWGILSSGGAYLPLSPEYPEERLRYMIEDSRTKVIFSQEGLETRLAELAPRGTRIVTLNDAAEFARAHTKLGKREPDTGPRPNNLAYIIYTSGSTGKPKGVMIEHRSIVNQMHWMKTVYKLNGEKVVLQKTPMSFDAAQWEILSPSCGSRVIMGGPGVYRDPGRLIETICRNEVTTLQCVPTLLQALLDTEELHRCESLTQIFSGGEALSRNLALQCLEAMPRCELVNLYGPTECTINSSAFTVDRTTVKNGPNTISIGMPVHNTQYYILDQHRAPVAVGEIGELFIGGVQLARGYLHRPDLTADRFIDNPFSTDPRHTRLYKTGDLAFWNADGSVQFAGRADNQVKLRGYRVELDEIRVAIETHDWVKNAAVIVKNDPRTGFQNLLSFIELNPKEAALMDQGNHGAHHQSKQSKLQVKAQLANMGCRDSAELIGKKVVDLPGKTPTQEQRRLVFARKTYRFFEGGDIKKEDILRLLGEQVTGTSSRGLEALSFADLGELLRYFGQYLSEERLLPKYGYASPGALCATQMYFELNQIGGLKPGYYYYHPVHHQLILIREKAGRATAQVKLHFMGKKRAIQPVYKNNIQEVLEIEAGHMVGLFEKVLPRYGLGIRELEYTPSTRDNLECADEDYYLGTFELVPYAHAGARPDDSLELYVQAHPGRIADLPAGQYQYKDGGLEKISDELVLKKHVIAINQRVYENASFGITVISKARKDWMRYVELGRKLQHLQMNDLQFGFMSSGYSSKSGNDLPSAKRMENILKACGREAGPSYFFVGGRVSQEQMLSEGMKEDSVHMKGPAELIKDDLVNFLPDYMVPNRVIILDKMPLTANGKIDFKALEKTNVELVERPFVAPRTAVEERISVLWKKEMKRDSVSVQDDFFESGGNSLIAVALINKLNKEFRTSLPLQVVFECPTIEKLALKIEGGNADPSSRLVRLQAEGTRKPIYCWPGLGGYTMNLRLLANKMGTERPFYGVQAHGINKDETPYPTIKEMAAEDIKLIKRLQPVGPYTLWGYSFGARVAFESAYQLEQSGERVEHLFLIAPGMPKVRAQDESLHGSAPTYENKAYVTILYSVFAGSITGPALDECLKVAKDEESFASFISGRFENLDPDLVKRIIKIVAQTYAFKYEFRELAERKINAPITIFKAHGDDYSFIENSSGYSAKAPAVMDLEADHYGLLKEPSIGELLKMIRY

SEQ ID NO: 2 - protein, 1292 aa, Photorhabdus luminescens
MKMLANNITQCDLITDACLKEDAITLMDMLENQLNHQADGYVVIDKEQSLNYADFYLKVKEIGYCLSEMSSKNSVGIGLFCDPSIDLICGAWGILSANKAYLPLSPDYPVERLKYMIEDSGIDVIFTQSHLKEQLQDIAPKSVLIITPEDVALTIETRTIEDILNTARVPNSTSLAYIIYTSGSTGKPKGVMIEHHSIVNQMRFLAKAFSLGKHSRILQKTPMSFDAAQWEILAPAIGSQVIIGPLGCYRDPDAIIKTILQHQVTTLQCVPTLLQALLDNPNFLDCLSLTQVFSGGEALTTKLAAQFLNSFTHCELVNLYGPTECTINSSYFRVTNETLPSYQTSISIGIPVDNTEYYVLDENRLPVAVGEIGELYISGVQLARGYLHKPEMTKDKFICNHLALETKHPWLYRTGDLVTRGDDGNTYFVGRVDSQVKLRGYRIELDEIRHAIEEHSWIKTAAMLIKKDARTGFQNLIACVELDEKEAALMDQGNSSSHHKSKTNKLQVKAQLSNSGCRSEELCENRPVILLPYKEGEIKQREYVFGRKTYRYFEGTEITIETLKTLLTTTQQREICSLPLSHLTLKDFGYVLRYFGQFTSHQRLLPKYAYASPGALYATQMYFELHHVFGLDAGIYYYHPVTHELIKISTLSRRQKPMIKVHFIGKREAIEPVYKNNIQEVLKMEAGHMIGLFDDILPEIGLGIGESEYQAECPDWYDGNIQDYYLGAFEICRYENRLPPFDTDLYLQTHTNKIPEMPCGLYHFSNGEFVRISDDIVRKKDVIAINQQVYDRSSFGVSIIPRCVPEWHYYITLGRRLHALQSNQLCIGLMSSGYSSESNNDLPSAKRMRSILNALDRPMAAFYFCIGGCVSQEQYISEGMKEDVVHMKGPVEIIKDDLHQQLPQYMIPNKVLVFDKLPLTANGKVDYQSLSKSKAVEDISTQRPLAPLRTETELRLGKIWMEVLKWDSVSAHDDFFESGGNSLMAVAMVNKINEAFNIHFPLQILFQSPNIEELAKWIEQADAKTISRLILLNQASQDPIYCWPGLGGYPMSLRLLANKVVPDRAFYGIQAYGINESEIPFSSIQRMAEEDIKEIKKIQPEGPYTLWGYSFGARVAFEAAYQLEQAGEEVKALNLLAPGSPHLHVNQEKYHDKGAEFTNPVFVQILFSVFARSISSPMVKTCLEQVNSEATFINFICSRFKNLDPSLVKRIVRIVTLTYDFKYSFDEIYHRYLKAPITIFKANKDNDSFIEKSGGFLSTPSKIIELISDHYQLLENEGVTEIEKITSFLNYQ

SEQ ID NO: 3 - protein, 1253 aa, Clavibacter michiganensis
MNANESFDSHDEVESLPVMLKAAVAADPGKAAVVAENTMMTYSQLWARATAQAEHLRRVGVRPGDYVGLYLEPGERLLVAVWAVLLADAAYVPLAVDYPSERIRYMIAQSAVAHIITDDVSASPARKLTPDGVHVHIAGEGTPTLAGAYDPDMSGSAYVIFTSGSTGKPKGVVIPHSAITHQMRWLAQEMALGQARILLKTPTSFDAAQWELLANAVGGTVIVGEAGIHRDPSRIRALVDSQHATHLQCVPTLWRALCREPEFARCESLTHVFSGGEALTPGDAKDIMQTLPHARLFNLYGPTETTINATSHRVGVDDVDPPDPVVTIGRPIPGCSVHILDAKRQPVALGDHGELAIGGPQVASGYLNDAARTEERFVTIEREGVATRVYLTGDIVSRDSAGALHFHGRTDDQVKVNGHRVETEEVRLAVEQHHWVRSAAVVPWKDPRDGVARLAAFVELDPHEAALMDADRAGQHHRSKKGHEQVLAQLAVLSRDGDRPDDVVLANAEGTPEQRRFAFGRKTYRFYEGGQLTVEDITRAVDTSQKPVPVAPLAALDVHVLAELLRWFGPFTSPERLLPKYAYASPGALNATTIYLEACGIEGLLDGMYEYRHASHGLRRVGPVESELPGPLRFHFVGSRRAIDSVYATNIDEVLHFEGGHMAGLLDHAAAQIGYSAQLQPSTQDARIVAAEHVCTAVIDLGPAAPAGDDLPVRVTVQMHDSVKDARSGTYDVGPAGLRRLSADIIERRHVIAINQETYDRSSFGVGLSVPSTAGWRGFVALGRALQRLQMNSEGIGLMSAGYSSLTGRDLPSALQYANIVGDASIMYFAVAGPVSEEQIRSTGMHEDSVHTRGPEEILRDDLRRTLPYYMVPSRITIVDEIPVSSSGKHDRAALVSVVEASVAPHAVVTPPKTDLERSLLDLWNETLRSDHRSVTADFFDLGGNSLDAVQLINRINRVLGVLLPVQTVFERPTVRGLAEVLSSRSLAMSRLIKLADGPGPVCVVWPGLGGYPMNLRAFAEHLSSKFTVYAVQTHGLNVGEVPYTTLREMIDRDIMLIDGALGDQDLAIAGYSFGARVATEVAATLTARGRAIKQVILLAPGSPFVPGLPLSSGDGFGDSYFRHMAYSVFVGRLPDDEAAAQLASMTDQESFLEALTLWRPGFDLELAARILAVASTTFGFRGDTVTDVDGLLAKMHVFAALGDTPSFLRSNEVPLEEHGQLRRLSVDHYAILRDPAVETVVGMLEARQVRVG

SEQ ID NO: 4 - protein, 1262 aa, Vibrio spartinae
MTLLGMFSEQISQNPDKIAVTDCNGSLSYLDFYLAVMNTGKYLADIQVSNENCIGLYCEPSIDMVCGAWGILASGNAYLPLAPEYPSERIRYMIQDSKVRVIYTQEHLKAQLEAVVPEHVTVVSGADIPLSFSDGTASLSNTVCSKITPENLAYVIYTSGSTGKPKGVMIQHRNIAQQMAFLKQCFSFGEHTRILQKTPMSFDAAQWEILAPVFGGHLFVGPSGCYRDPDVMVEALLKYDISVLQCVPTLLQALIDHPLFVDCKELKQVFSGGETLTRQLSKEFYGVRPESELINLYGPTECTINSSYFRVCSEELDNYPSAISIGKPVVQTQYHILRNDGQPTLVGEIGELYISGPQVAKGYLHRPDLTEDKFVPNHISKDPGHSRLYRTGDLAHCDGEGNVHFAGRADNQVKLRGYRVELDEIRHAIEKHLWIKNAAMVINNDARTGSQNLIACVELDKTQAAIMDQGNHGAHHQSKANKHQVKAQLSNAGCRQSEECVGKLKIELKGKDATDRQKEKAFGRKTYRFFDRHHPVTKEELTVLLTFEQPKSLSCDVAQLTESQFGELLRHFGQYISDERLLPKYAYASPGALYAAQMYLELNGLFGWPAGIYYYHPIEHCLIKIKTLVQSEVPVFKLHFIGKRDAIEPVYKNNILEVLEMETGHILGLFDELLPNLGLSIGRHHKLEQLPSWYDGKKHDYNLGTFEICSNESAPEIEPPELYIQVHSNRVLGLTEGLYRFVNQDFEYLSDQLIMKRDVIAINQEVYDRASFGISMVENTSEQAMRYISLGRTLHKIQSNPLLLGVMSSGYSSKTGNDLPSAIRMRQILNTQGKNLKAFYFCIGGGISEEQYFSTGMKEDTVHMRGPTEIIRDDLMSQLPQYMIPNKVVIIDKLPQTANGKVAYQALKALDVVVNCGSEKKFVPLVTETEKQLGEIWCRIMKWNTASAEDDFFECGGNSLTAVAMINRINQTFEIKVPLQVLFKSPTIKQLAKWIDSQDEHTQSVSRLIELNNAQQRPVFCWPGLGGYPMNLKLLANQVSPERQFFGVQALGINEGEIPLSNVQEMAKADIELIKAVQPDGPYTLWGYSFGARVAFETAYQLEQMGETVEALNLIAPGSPQTHCDLEQMNQGEASFSNPVFVTILFSVFAHTIDGALLQSCLNQCHDEDSFVKFMCKRFPVLQEDLIRRITRIVQVSYNFSYTFEELVGRKINTPVTIFKAQQDNYSFIESAPAFSKVPPKIVNLQVDHYQVLKEQGVAELK

SEQ ID NO: 5 - protein, 1264 aa, Arthrobacter antioxidans
MTSVPAPKTLTEMLRYQVAAHPQAVASIDADGKLTFEELLSRADFAAAALTAAGVGPRDTIGLFMAPSNHLLIATWAILSADASYLPLAVDYPAERISYMISDSKIRHVVVDGGSYELIEHLVPEHVQVLRIEQLFQAGPKRDYSAYTEDLPAYVIYTSGSTGRPKGVVIGHPAIVNQMQFLAESAGLNPGERILLKTPISFDAAQWELLANAVGATIVVGPHGVHRDAELISNLVLEYEVSLLQAVPTLWTALLETGKLTRCRSLRGLFSGGEVLPSRLARELLATLPQAQLVNLYGPTETTINATWLRLSIGDIPDTPAVSIGSAVPGCQVHILDAELRPVLDGQTGELCISGSQLAYGYLHRPRLTAERFVEADVDGRPVRLYRSGDVARIDASGQVEFCGRIDDQVKVNGHRVETDEVRLAIEEHHWVRQAAVVPWRSPEDATIRLGAFLELDPAEATLMDQDAAGEHHRSKASHVQVRAQLAGLGVRNFTHPQRSVVLPGREGTPEQFQAAFARKTYRFFEGLPISAADLVALGESLDQPWAIGPRSGLVTISDVADVLRWMGPFPSSQRLLPKYAYASPGALNATQIYLETSGMPGLDAGMYYYHPTKHELIHVGHQAARLSAPSLRLHLAGLPRVIESVYSTNVVEVLHMEAGHMVGVLDRAAAARGLRLQPVPSVDLPELGLTEKWESTGVFDLIECVDAVPSAPLVDLTFQIHGKVAGARHGTYCLKDGTLHRLSNHVIERRQVIAINQGTYDRSSFGVTLSCRREQGWSGFFELARALQKLEMNEQGIGLMSSGYASLSGRELPSARRYDQIARAWGQDESRLSYFAVGGSVSAEQVASTGMKEDAVHLRGPEEIVKDDLRRILPHYMVPSRVEVLDKLPMSSSGKVDRAALIQQLESDQRPQRAVILPSSKLETDCLNVWSRMLDREVLSVSDDFFDIGGNSLTAVKLINALNEHFGSSLPIQSIFEAPTARELAERIRQVAPANASRLVLLAPGTGEPVFVWPGLGGYPMSLRGLAARISHGRPVYGIQAYGLNPGESPCTTMEEMVAADVEPVLEAAEGNSVTLMGYSFGARVAAEVALWLEARGVTVDRLTLIAPGSPEIDGVPDRPWNGPNAFLDPYFRMILMSVFTGSLDGLHRDALLAKVRTRNEFMELLAGRVPALDMALASRIIEVVAATYSFRSQPASDIQQVLSQTQVLIAAGDGPSFASPYRDRMEARGAVVELEANHYQVLHEPTVDLTANFIMALQEQPA

SEQ ID NO: 6 - protein, 1488 aa, Dickeya dadantii
MDNISNHAFNYPVLVLNKGLLPEHDDIALARYLFSALALAVSRITQNEEMIVGFHLHPQEITRWKDDECIRQYILPLNIRFNSATPIAGFIREIMTWMTPDAIHQKNAMGASVLTLGPQHALHDIFDLEISWQPPVESEPVQALTCHVASREDALVLTLRFNPARFSATQMQKLPEVWRQITASAAKNGAETLRDIGLIDDAERQRVLHAFNQTEQAWDGETTVAARLKNRAQRHPEQTAVVFRDETLSYRQLYQQAGALAHYLNALETERERCVGLFVEPSLTLMTGVWGILLSGNAYLPLSPEYPEDRLAYMLENSQTRIIVTQPHLRERLLALAPPGIQVVTSDDVDAFMRQHAHSLPDAPQNDIAPHHLAYVIYTSGSTGKPKGVMIEHHSVLNQMNWLAQTVGLNQETVILQKTPMSFDAAQWEILSPACGCRVVMGEPGVYRNPEQLVDMLAEYRVTTLQCVPTLLQALLDTERLTHCPALRQIFSGGEALQKHLAQACLETLPDCELINLYGPTECTINNSAFRVDPVSVRQGPDTLSIGAPVANTRYYILDNCLTPVAVGQIGELYIGGDGVARGYLNRDDLTAERFIVDPFAPAGSGRRLYQTGDIASWNPDGTVQYAGRADNQVKLRGYRVELDEIRSAIETHEWVKAAAVIVRNDPFTGYQNLISFIELNAREAALMDQGNHGSHHQSKADKAQVMLQLANKGCREFPAASQPYTLDLPGKQPDEKQRLTAFSRKTYRFYDGGAVSREDILSLLHEPLLTAISRQPDALTLDELGHWLRYLGQFTSAERLLPKYTYASPGALYATQVFLELNGVAGLTAGHYYYQPVHHQLVRVSEQAAVTPGSLRLHFVGKKSAIEPIYKNNIREVLQMEMGHIIGMLDIILPDYGLGVALCDAAALDPTPLAIDLDDDYLGACDVLSGPRLPTDDDLDIYVQTAGANIADLPVGTYRYVRGDLQHIADDVIDKKHVIAINQAVYERSSFGISVASRTEGWAGYVHVGRKLQRLQMNPLNIGLMSSGYSSETGNDLPAARRFWQILGHRTGPYYFFIGGRISDEQKYSEGMREDAVHMKGPAEMIRDDLAAFMPDYMMPNKVLILDEMPLTANGKIDMKALANINVELKHKTIVAPRNPLEHQVMAIWQAKLKREEMSVDDNFFESGGNSLIAVSLINELNATLNASLPLQVLFQAPTVEKLAAWLSRARREPVSRLVQLQPKGRQAPIYCWPGLGGYCMNLRLLARQLGAERPFFGIQAHGINPDETPYATIGEMAARDIELIRQHQPHGPYTLWGYSFGARVAFETAWQLELAGEVVENLYLLAPGSPKLRDERVAAMNRKADFDNPGYLTILFSVFIGSITDPELERCLETVRDEESFVAFITGLNPALDDGLVRRITRIVAQTFEFTYTFSELQQRQLNAPVTIIKAQGDDYSFIENHGGFSAQPPTVLELMADHYSMLKAPGIDELTSVIQYQQSPPSLVG

SEQ ID NO: 7 - protein, 1284 aa, Photorhabdus laumondii
MLENNITQCDSINDVYLKEEAITLMDMLESQLKHQADGYVVIDQEESLSYADFYLRVKEIGYCLSEISSKNSVGIGLFCDPSIDLICGAWGILSADKAYLPLSPDYPTERLKYMIEDSGIDVIFTQSHLKAQLQDIAPKSVLIMTPEDVALTIKTRTIEDILGTVQVPKPTSLAYIIYTSGSTGKPKGVMIEHHSIVNQMRFLAKAFKLGCHSRILQKTPMSFDAAQWEILAPAIGGQVIMGPLGCYRDPDAIIKTILQHQVTTLQCVPTLLQALLDNPNFLDCLSLTQVFSGGEALTTKLATQFLNSFTHCELINLYGPTECTINSSFFRVTNETLPNYQTSISIGAPVDNTEYYVLDDDRLPVAVGEIGELYISGAQLARGYLHKPEMTKDKFICNHLVSGTQHQWLYRTGDLVTRGADGNTYFVGRVDSQVKLRGYRIELDEIRHAIEEHSWIKTAAMLIKKDARTGFQNLIACVELDEKEAALMDQGNSSSHHKSKADKLQVKAQLSNSGCRSEELCENRPTFLLPYQEGEIKQREYAFGRKTYRYFEGTEITVEKLKKLLTATQSNEISSLPLSHLTLNDFGYALRYFGQFTSHQRLLPKYAYASPGALYATQMYFELHNVLGLDAGIYYYHPVTHKLIKISTLSRRQMPTIKVHFIGKHEAIEPVYKNNIQEVLEMEAGHMMGLFDDVLPEIGLSIGKSEYQDECPDWYDGDIQDYYLGAFEICSYEHGLPPFETDIYLQTHAHKIPEMPCGLYHFSNGEFVRISDDIVRKKDVIAINQQVYDRSSFGVSIIPRCVPEWHYYITLGRRLHALQSNPLYIGLMSSGYSSKSNNDLPSAKRMRSILNALDRPMAAFYFCIGGGISQAQYMCEGMKEDVVHMKGPVEIIKDDLQQQLPQYMIPNKVLVFDKLPLTANGKVDYQSLSESKAVENVSTQRLLVPLHTDTEIRLGKIWMEVLKWDSVSALDDFFESGGNSLMAVAMVNKINAAFNIRFPLQILFQSPNIAELAKWIEQTDSKTISRLILLNQASKDPIYCWPGLGGYPMSLRLLANKVVPDRAFYGIQAYGINESEIPFSSIQRMAEEDIKEIKKIQPEGPYILWGYSFGARVAFEVAYQLEQAGEEVNALNLLAPGSPHLDMKQAEYMDKGAEFTNPAFVKILFSVFSRSINSPMVKTCLEQVNSETTFINFICSRFKNLEPSLVKRIVRIVTLTYDFKYSIDELYHRHLKAPITIFKANRDNDSFIEESDVISSMSPKIIELISDHYQLLESEGVAEIEKII

SEQ ID NO: 8 - protein, 1288 aa, Vogesella indigofera
MSAQMNTLQAFLQQHIQSSYTHTSPYLRTLPAMLLAQVHSRKNEIAIADQETEYSYETTLLHAVGIAAALRAKGVSYNDCLGLFIDASADLALATWGILFAGAAYLPLATDYPQDRLSYMVSDARVKLVLTNNRSRERLAGVLLPGVSLLNIDDVAPASITEVDQVLTELLEQDGEDLAYVIYTSGTTGKPKGVAISQQAIANQLAWLTHEGYLLPGHRILQKTPVSFDAAQWELLGMCCGARVVMGQPGVYRDPEALIRQVQQHGITTLQGVPTLLQALSELPAFADCDTLHSLFSGGEGLSRKLASKLLQILPDCRLVNLYGPTECTINATHYRIDQRSLDQEWEIAPIGLPVAGLHYQVLDLQLAPVAAGQTGELFIGGAQLANGYLFRAEQTAEKFLSLPLDAASPQVRMYRTGDLVRVDPDGVLHFVGRTDNQVKFRGYRIELDEIRLAIENHDWVKSAAVFIKENARSGHAQLIGAVELNPNEAKLMDQGAAESHHQTKASRHQIKAQLSGRGFRSDASLAGRQQIALPGQQETAAQRERVFARKTYRFFHGGPVAATDILQLLAAQPEAAPSARLADLDASKLGEMLRYLGQFSSEERLLPKFGYASPGALYATQVYIELNQVADLPAGYYYYHPLQHRLYLLAASSATEPCLRLHFVGKIPAVREVYKNNILEVLEMESGHILGMLDHVLPGYGYGLGLGRHTPEVLARLDCPEEHDYLGSYDIVALAERIDDLAVDIYVQAQQGRIHDLADGNYLYRQGRLDKVSGHLIEQRHVIAINQQVYQRASGGLSFVSQSQAPWRQYLDLGRSLQRVQMNRLGVGTMSSGYSSKSGNNLATALRLNDIVGDAGRESGPSYFCLFGKVSAAQLSHQGMDEDAVHMKGPAELIRADLLNSLPDYMVPGRIAIINQMPHTTSGKVDVQALKQCADFQLADQEQPHVAARTATEQAISQIWCHILQLEEVSVMDDFFALGGNSIQAVAIARAINRQFAAKVPIQLLFSAPTIEKLAQAVSGGDMQTASRAVALAGHDGTLATFCWPGLGGYPMNLRLLGEAVAGERRFYGIQAHGINAGETAFDSIEAMAREDVQLLRSLQPQGPYALWGYSFGARVAYEVAYQLEQQGETVSQLVLLAPGSPRLSHAEPVSRDERELFANPAFLTILLSVFAHDIDPALNADCLLRVDSRATFIRFAAEKFPAIDRTLIDAIANVVAVTYSPDYQIALADKPLCCPISVWRARGDGPSFIADGEQAGLRIRWHDLDVGHYAVLKAEGIAAMQAAVLA

SEQ ID NO: 9 - protein, 239 aa, Dickeya chrysanthemi
MFSSFIKDIEWLTIYQAGNTAVYPGHCARCHFDLSAYRDELFAVAGLPVPAEIARSVPKRRAEYLAGRYLAQTVLSRLGVAGYVLTSARDRSPQWPQGIAGSLSHNADSVLCAAHPCNQAMTCVGLDIETRMSAERADNLWPGIADEIEYDWLHSHDPISFASMLTLSFSAKESLFKALYPQVKRYFDFLDVRMVELDTVQQTFTLQLLIDLSPDYLMGCRFSGAYQLRESDITTFLFN

SEQ ID NO: 10 - protein, 247 aa, Photorhabdus laumondii
MEQTVIHTNQASLPEYDNGFITQIEYGTVVKHPQVHFWHARFDLSYYHDELFEQLNLPFPATLTKAVKKRRAEYLAARYCARQLLAQLGQPAFNLISGHDRAPIWPQNICGSVSHSSHCAIVLAAPQTHNRLIGVDIEAIVDRQNIDEITKMIVNDKEIQLLKHCHLPLEQAFTLAFSVKESLYKALYPQVKRFFGFEAAEIIALSLENNEITLALRETLTPHYPAGTLFRGQFIIYPQEILTLIIQ

SEQ ID NO: 11 - protein, 233 aa, Pseudomonas putida
MNTLPACCAPLRHHWPLPRPLPGAVLVSCAFDPAHLATDDFQRAGIVPSASLQRSVAKRQAEYLAGRVCARAALQHLDGRDYVPGTHEDRSPIWPAGIHGSITHGKGWAAAVVAGENSCQGLGLDQEALLDDERAERLMGEILIPAELERLDRRQLGLTVTLTFSLKESLFKTLYPLTRQRFYFEHAEVLDWSAEGLARLRLLTDLSPQWQQGAELQGQFCLQDGHLLSLVSV

SEQ ID NO: 12 - protein, 224 aa, Bacillus subtilis
MKIYGIYMDRPLSQEENERFMTFISPEKREKCRRFYHKEDAHRTLLGDVLVRSVISRQYQLDKSDIRFSTQEYGKPCIPDLPDAHFNISHSGRWVIGAFDSQPIGIDIEKTKPISLEIAKRFFSKTEYSDLLAKDKDEQTDYFYHLWSMKESFIKQEGKGLSLPLDSFSVRLHQDGQVSIELPDSHSPCYIKTYEVDPGYKMAVCAAHPDFPEDITMVSYEELL

SEQ ID NO: 13 - protein, 229 aa, Clavibacter michiganensis
MLDAILPAGAVLAEERGPAGEHPLHPAEAGAVARAVPSRRREFAATRACARTALAALAAFDGDAPGAAPVAIPKGRGGDPVWPRGVVGSLTHCAGYRAAVVAGVDALRTIGIDAEPHAPLPREARDVVGLAGELDPHPPLGADVHADCVLFSAKESVGKAHYARYREWLGFADLHVTLHPGGAFTARRCAPGPVPFPAYRGSWRVAEGLVLTCAWLAVPRVPSAVPRPA

SEQ ID NO: 14 - protein, 226 aa, Clavibacter michiganensis
MTHGFRALLPSAVEVEVATDDADGASPFDAERAAVARAVPVRRAEFFTVRACARRALARLGQPPVAIVPGPGREPVWPAGVVGSMTHCRGRRAAAVARSDEILVLGIDVELHAPLPEGVAELVMSPAERGRLDELGRIQPSVAWDRVVFSAKESVFKAWYPLARSWLDFLECDLELDARTGTFDARLLVPGPLIGLDRLQVLRGRWATEGGFITTAVWEAAAPTRR

SEQ ID NO: 15 - protein, 226 aa, Streptomyces albidoflavus
MLKTILPEGLSVVETRADVPESTLYAEEAAVVARAVHKRRAEFTAVRHCARQAMAELGVAPAPLLPGERGAPRWPDGVTGSMTHCDGYRAAVVGLASRFRSVGVDAEPHDTLPDGVLGTIALPSERERHTALRRDRPDVHWDRLLFSAKESVYKTWFPLTGRWLDFEEADITLAPGAGHTGTFTARLLVPGHTTSGDPLTGFEGRWTVAEGLVCTAIALPAPVPRP

SEQ ID NO: 16 - protein, 229 aa, Streptomyces avermitilis
MIEELLPAAVVAVEAHGDEAAVDGALYPEEQAVIARAVEKRRREFTAVRVCARRAMEKLGVPPQPVLPGERGAPRWPAGLVGSMTHCEGYCAAVLVRAGELASLGIDAEPHDRLPEGVLSSVALPTEERRLYDLGRSRPDVHWDRLLFSAKESVYKAWFPLTGKWLDFLEADIEIFTEPAAQGKALSGGFRAELLVPGPLVNGRRLDAFDGRWTVRRGLVATAVTVPHH

SEQ ID NO: 17 - protein, 255 aa, Streptomyces clavuligerus
MIESLLPPEAVWAEHFGPDPSARLLPEEEPHVAQAVARRREEFTTVRACARRAFAALGLPPGPVLPGVRNAPRWPAGVVGSMTHCDGYRAAVLARATDLAAIGIDAEPDLPLPDGVLEAISLPGERARLGLGGPAAPRPHGGRTVCRDRLLFSAKEAVYKSWYPLLGTELDFHEADISFHGGPDGTERAGGTERADSTVRTEGTGGVSGTFTARILRPEREPGGRLVEEFTGRWLSERGILVTAIGVPAPRTVPR

SEQ ID NO: 18 - protein, 229 aa, Streptomyces clavuligerus
MATESPADSRSVTTPAPPPGVELVWYGRVPALAADALAHRGLLDAGEQARLDGFLRPRDRDAYAVAHVALRRLLGERLGLPPGAVVVERRPCLHCGGPHGRPVVAGDPVHFSLSHTTGAVLIALARTAVGVDIERLPSPASVDDIADQLHPGERAGLAALTGEERVRAFARCWTRKEAFLKATGAGLTEDLSRTLVGAGPRPAEVPGWSIADLAADAGYTAAVAVQTPQ

SEQ ID NO: 19 - protein, 226 aa, Streptomyces lividans
MIEELLPGTVVAVEAFGQDDAGHLPLYPEEEELVARAVAKRRREFTVVRSCARRAMEKLGVPAQPVLTGERGAPRWPEGIAGSMTHCDGYGAAALVRLTDLASLGIDAEPDGPLPDGVLESIALPAEVALLRRLGGARPGVHWDRLLFSAKESVYKAWYPLTGQWLDFAEADIEIRVDPADPRRGTLHAALLVPGPTVDGRRLSRFDGRWSARDGLVTTAVTVPRT

SEQ ID NO: 20 - protein, 231 aa, Streptomyces venezuelae
MTSEISPVRVLRAGAGPDPSVPPVVDGSVALWLVPVTADPGAYELLDAGERQRSAALLREADRTRYLAAHGGLRRLLGHYLGTPPDEVVFVREDCPLCGGPHGRPAVRDGGIHFSLSHSEDLVLVGLAGRPVGVDVEAFPAAGVSDLVADTLHPREREEFARLAPEVRTAAFTRCWVRKEAYLKGTGEGLAGGLERTYVGTGPGPAAVPGWSLTDVPVGHSYAAAVALSQQ

SEQ ID NO: 21 - protein, 254 aa, Vogesella indigofera
MSGAVVPAGLLSALRHVLSPPDDAGASVAALWRPWPLPVAGGSVAALSLPALDALLGDIVPASLHPAIPGRCVRSRQLSFLAGRLCAEQALAALPLTAAVLGRDASGRPLWPAGVTGSITHSPTLAAAVAAWSPQTDGLGVDCEPLAAGERLDDIVSACCTAADRACLPSRAAGTMATLIFSAKEAGYKALSHRFGRIVDFTEFEVCQLARDAGQLWLAPVPGSEWHRRIQPFAVQFTFDADSVYTLADLRGIP

SEQ ID NO: 22 - protein, 76 aa, Streptomyces lavendulae
MSESQNDERAYRVVRNDEDQYSIWWADRDLPAGWHAEGTEGPKDACLQRIEEVWTDMRPLSLRRRMQQQDQALIAI

SEQ ID NO: 23 - protein, 74 aa, Archangium violaceum
MSNQDTEDTQKYKVVVNHEEQYSIWPADRENALGWKDAGKEGSKAECLDYIKQVWTDMRPLSLRKKMEEAAKGS

SEQ ID NO: 24 - protein, 71 aa, Myxococcus
MTDEREDTTVYKVVVNHEEQYSIWPADRENALGWKDAGKQGLKAECLEYIKEVWTDMRPLSLRKKMEELKS

SEQ ID NO: 25 - protein, 291 aa, Vogesella indigofera
MPASRLWRDTLLTAVVPLIWGSTYLVTTQWLPAGLPFSAGVLRVLPAGLLLLLWTRHLPQRQEWPRLLLLSLLNIGAFQALLFVAAYRLPGGIAAVLGAIQPLLMMGLLWGLERQTPRPLVLLAACAGLAGMAVLLNAGNARWDSIGVLAAAGGAVVMALGMYLARRWGSSLPLLALTGWQLALGGLMLLPAALWLDPPLPALSAANVAGYAYLSLVGALLAYGLWFRGLRLLPPVAVSALGLLSPVAAVILGWVALGQRLHGWTLIGMVTVLASVLCVQLASSRRPARAG

SEQ ID NO: 26 - protein, 297 aa, Dickeya dadantii
MKLKDFAFYAPCVWGTTYFVTTQFLPADKPLLAALIRALPAGIILILGKNLPPVGWLWRLFVLGALNIGVFFVMLFFAAYRLPGGVVALVGSLQPLIVILLSFLLLTQPVLKKQMVAAVAGGIGIVLLISLPKAPLNPAGLVASALATMSMASGLVLTKKWGRPAGMTMLTFTGWQLFCGGLVILPVQMLTEPLPDLVTLTNLAGYLYLAIPGSLLAYFMWFSGLEANSPVIMSLLGFLSPLVALLLGFLFLQQGLSGAQLVGVVFIFSALIIVQDISLFSRRKKVKPLEQSDCVIK

SEQ ID NO: 27 - protein, 306 aa, Archangium violaceum
MRGRSTDWLLAMAGGALLALMINYNSLLAKHTTPVFASWVAHGLGAVAAFALVVLYSRLFRSPGKEMEPQRAKVPLWFYLGGIPGTLTVILAAVTVNSGLSLSGTIALMLLGQVLFGLVSDYFGLFRTPKRRIVATDFLVALCILSGSALIIFGRAPMIAYILLAFFNGTVIGTSRAINGRLGAEIGSVKASLWNHLIGFLFLTPALLLLGGWKFEVIPEAPASAYIGGFFGALFVAVNSYVFPRLGAMNASLLVISGQMISAVLLDYRNQGVAPTVTRCLGVVIVLFGVYLTRAAKNPRVKDKSQ

SEQ ID NO: 28 - protein, 149 aa, Archangium violaceum
MIAYILLAFFNGTVIGTSRAINGRLGAEIGSVKASLWNHLIGFLFLTPALLLLGGWKFEVIPEAPASAYIGGFFGALFVAVNSYVFPRLGAMNASLLVISGQMISAVLLDYRNQGVAPTVTRCLGVVIVLFGVYLTRAAKNPRVKDKSQ

SEQ ID NO: 29 - protein, 392 aa, Photorhabdus laumondii
MINKLLNDRSYHIEFNGHLTNHIKHAVIALHGLGISASRIKDYYDNYVKLTPYGMGLEPPKTLKHVVDSSNWKHFLGKRTSYSSYCDYFEEEIKQKGIEQVLQEYMPTLLSGWAGALTHGTIHLGWALDINHPWMIIEGLAYMAFSYVPCHSERAFIDSSLNDKNAFDSILRISTLWEQRRAELTDWVNSLIDNEDLADTDLIHPELKRSGLQYRIARILMQGHPEIYRLPVWIETQNVEESWAQLFYVVTLIYLAKPGDFLFLHLITSLFAMKNIASRLPAEESKNIIKCYWVGMLCILFSTTDLSKPAKFSALNQTYSFRQDEMTYPVWEQEWSHIIARALEEEEEHNPKLVYVMNQLWKEQGLTIYRAASSQFTSTPLLPPSFEEAPIE

SEQ ID NO: 30 - protein, 408 aa, Vogesella indigofera
MTQHTKPAGVLLLLAILLLSLNLRPAIAAIGPLIQSISHDAGVRSVGISLLTTIPVLMMGLGAIYAARIRQALGERGGITAGSALIAVACLMRLWADDRNGLFLSAALVGLGIAVIQALLPGFIKRNFGARTSSVIALYSTGIVAGAAIGSGTASWLEDQLGWLSTLASWSLPAALATVFWLLASLGSQHEGRSNVQATTRSVPFWRIGRCWSLLLFFGIGTGAFMLAMAWISPFYIGLGLGKATAGLLLSVLTIVEALTALWLSFAIGRFPDRRGLLLTSLLLVAAGFLLLIVAPLLAPYFAVSLLGVGIGILFPLSIIVAMDHLKDPTLAGNFTAFVQGGGFIIASLVPLTAGSLRDVFNDLSYIWLLMSIASLGMLVLAVRFSPASYQQFQQDLAELQAGELKPA

SEQ ID NO: 31 - protein, 158 aa, Photorhabdus laumondii
MINTIRNILLAVIGGSLLTLMIYTNSMLSESTTPFFASWVAHGIGAIVALILFIIVSKLFSRKEIDESKHKKSNIPIWFYLGGIPGAFTVVLAAIAINGGLPLSSTISLGLVGQILFGLVADRFGFLGTRKRKIVIQDFYVIFFVLFGSILILFGGSN

SEQ ID NO: 32 - protein, 145 aa, Photorhabdus laumondii
MLSLIFIAFLNGIFIAATRTINGQLSVSIGSFGASLWNHIGGFFLLTVILTLLSSWQWNKWDFSVIPTAAYMGGVFGALFVAVSSYIFPKLGAMNAAVLVIAGQMLSAVLLDWLYQGMTPGLVKIVGVLFVLIGIYLTRKTTELK